(19) United States
(12) Patent Application Publication
(10) Pub. No.: US 2007/0122795 A1
(43) Pub. Date: May 31, 2007

(54) Information processing apparatus, information processing method, information processing system, program, and recording medium

(76) Inventor: Tetsuya Shiraishi, Tokyo (JP)

Correspondence Address: William S. Frommer, Esq.;c/o Frommer Lawrence & Haug LLP, 745 Fifth Avenue, New York, NY, 10151, US

(21) Appl. No.: 11/521,243

(22) Filed: Sep. 13, 2006

(30) Foreign Application Priority Data
Sep. 14, 2005 (JP) 2005-266728

(51) Int. Cl. C12Q 1/00; G06F 19/00

(52) U.S. Cl. 435/4; 702/19

(57) ABSTRACT

Disclosed herein is an information processing apparatus, method, system, and program, and a recording medium. An information processing apparatus includes an acquisition unit, an arithmetic unit, and an output control unit. The acquisition unit acquires the amount of the molecules for detection which have been produced by control cells and sample cells. The arithmetic unit receives from the acquisition unit the information about the amount of the molecules for detection which have been produced by the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection. The output control unit controls the output of the score which has been calculated by the arithmetic unit for the cellular function.

### CROSS REFERENCES TO RELATED APPLICATIONS

The present invention contains subject matter related to Japanese Patent Application JP 2005-266728, filed in the Japanese Patent Office on Sep. 14, 2005, the entire contents of which being incorporated herein by reference.

### BACKGROUND OF THE INVENTION

1. Field of the Invention

The present invention relates to an information processing apparatus, method, system, and program, and a recording medium. More particularly, the present invention relates to an information processing apparatus, method, system, and program, and a recording medium, which are intended to digitize the relation between intermolecular interactions and cellular functions.

2. Description of Related Art

There are many diseases involving gene defects. They include genetic metabolic diseases induced by a single gene defect as well as cancerous diseases induced by a plurality of gene defects which have accumulated with time. Analyzing whether a specific gene (and its product) is normal or abnormal is important in understanding the origin of a disease and establishing the plan for medical treatment.

This has been generally achieved by the technique which involves investigating the copy number of a specific gene of interest, confirming the degree of transcription of the gene, performing DNA sequencing on the amplified product of RT-PCR of the gene, detecting mutation by the thus obtained base sequence, and confirming immunohistochemically the localization of the gene at the protein level or the change in expression of the gene. This technique has helped accumulate a large amount of knowledge, some of which is used as an essential method for clinical test.

The copy number of the gene may be investigated by using the Southern blotting technique, which involves treating a sample with a restriction enzyme, transferring the treated sample to a nitrocellulose membrane by electrophoresis, and hybridizing the transferred sample to find a specific base sequence. The degree of transcription of the gene may be investigated by using the Northern blotting technique, which involves separating RNA by gel electrophoresis, transferring the separated RNA to a nylon membrane, hybridizing the transferred RNA with a labeled probe, and detecting the desired molecules.

These classic techniques of the first generation are followed by the new techniques of the second generation, which are designed to examine a very large number of genes and proteins comprehensively at one time. They have been developed for the human genome project, which needs to examine a large number of genes comprehensively at one time. Nowadays, comprehensive analyses are carried out not only for genes (genome) but also for RNAs (transcriptome), proteins (proteome), and metabolites (metabolome). Many methods have been devised to utilize the resulting data for disease diagnosis and medical treatment.

There have been proposed many methods for studying how a change in mRNA affects a disease by computer analysis of comprehensive data originating from transcriptome which is a collection of mRNAs or all of transcription products in a cell. Among them is a method for knowing the property of cancer and devising the medical treatment of cancer by analyzing the expression profile of mRNA and other molecular data. For example, refer to following Patent Documents 1 to 9.

Patent Document 1:

Japanese Patent Laid-open No. 2005-34151,

Patent Document 2:

Japanese Patent Laid-open No. 2004-329211,

Patent Document 3:

JP-A-2005-514051,

Patent Document 4:

JP-A-2005-512557,

Patent Document 5:

JP-A-2005-514359,

Patent Document 6:

JP-A-2005-518522,

Patent Document 7:

JP-A-2005-500832,

Patent Document 8:

JP-A-2005-503779,

Patent Document 9:

JP-A-2005-508199.

There has been disclosed a technique for a drawing a graph that shows nodes representing proteins and edges representing their interactions and then visualizing it three-dimensionally by using a parameter called spring force. (For example, see Patent Document 10: Japanese Patent Laid-open No. 2004-118819.)

There has also been disclosed a technique for visualizing by means of nodes and links a table that shows interactions and their intensity between objects such as proteins. (For example, see Patent Document 11: Japanese Patent Laid-open No. 2004-30034.)

### SUMMARY OF THE INVENTION

Unfortunately, the techniques disclosed in Patent Documents 1 to 9 above are designed to analyze comprehensive data, abstract their feature, and find their relation with specific diseases. They do not give any information about how a set of data of gene increase or decrease relates with specific diseases. Even though they provide the relation between the presence of gene expression cluster and the clinical data, they do not indicate the significance of the relation. Therefore, they do not permit one to ascertain the difference between meaningful data fluctuation and meaningless data fluctuation due to sample preparation for comprehensive data.

In addition, the techniques disclosed in Patent Documents 10 and 11 make intermolecular interactions visual but have no way of digitizing and predicting intermolecular interactions.

The present invention was completed in view of the foregoing. It is intended to digitize the relation between intermolecular interactions and cellular functions.

The first embodiment of the present invention is directed to an information processing apparatus which includes acquisition means, arithmetic means, and output control means. The acquisition means acquires the amount of the molecules for detection which have been produced by control cells and sample cells. The arithmetic means receives from the acquisition means the information about the amount of the molecules for detection which have been produced by the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection. The output control means controls the output of the score which has been calculated by the arithmetic means for the cellular function.

In the information processing apparatus, the acquisition means acquires the amount of the molecules for detection which have been produced by the control cells and the sample cells, according to the amount of the nucleic acid which has been expressed in response to the molecules for detection which have been collected from the control cells and the sample cells.

In the information processing apparatus, the combination of the two molecules for detection is classified into the following five categories according to the interrelation between the two molecules; the first category applicable to two molecules which suppress each other, the second category applicable to two molecules the first one of which promotes the second one and the second one of which suppresses the first one, the third category applicable to two molecules which promote each other, the fourth category applicable to two molecules only one of which promotes the other, and the fifth category applicable to two molecules only one of which suppresses the other.

In the information processing apparatus, the arithmetic means calculates the score for the cellular functions by accumulating for each cellular function those values which are obtained by giving the score based on the amount of the molecules for detection which have been produced in the control cells and the sample cells to the cellular functions relating to the mutual promotion or suppression between the two molecules for detection which belong to the first to third categories out of the five categories and then multiplying a prescribed factor.

In the information processing apparatus, the prescribed factor is established such that it takes on the largest value for the cellular function relating to the first category of the first to third categories out of the five categories and it also takes on the smallest value for the cellular function relating to the third category of the first to third categories out of the five categories.

In the information processing apparatus, the prescribed factor is larger than 1 when the two molecules for detection have a molecular bond.

The information processing apparatus further includes storage means that stores in a table form the information about the combination of the two molecules for detection which are classified into any of the five categories and the cellular function relating to the mutual promotion or suppression of the two molecules for detection.

The information processing apparatus further includes estimating means that estimates the score for the cellular function when there is any change in the amount of the molecules for detection which have been produced in the control cells and the sample cells after it has been acquired by the acquisition means.

The information processing apparatus further includes network building means that builds a network for the information about the interrelation of the molecules for detection, so that the estimating means calculates the effect of change in the amount of the molecules for detection which have been produced on other molecules based on the network which has been built by the network building means, thereby estimating the score for the cellular function.

The information processing apparatus further includes analyzing means that analyzes the change with time of the cellular function based on the score for the cellular function, with its output being controlled by the output control means.

The second embodiment of the present invention is directed to an information processing method or an information processing program which includes the steps of acquiring the amount of the molecules for detection which have been produced in the control cells and the sample cells, receiving the information about the amount of the molecules for detection which have been produced in the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection.

The third embodiment of the present invention is directed to an information processing system which includes an analyzing unit that analyzes the amount of the molecules for detection which have been produced in the control cells and the sample cells and an information processing apparatus that analyzes the information about the cellular function relating to the mutual promotion or suppression of the two molecules for detection. The information processing apparatus has acquisition means, arithmetic means, and output control means. The acquisition means acquires the amount of the molecules for detection which have been produced by control cells and sample cells. The arithmetic means receives from the acquisition means the information about the amount of the molecules for detection which have been produced by the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection. The output control means controls the output of the score which has been calculated by the arithmetic means for the cellular function.

The information processing system includes the steps of acquiring the amount of the molecules for detection which have been produced in the control cells and the sample cells, receiving the information about the amount of the molecules for detection which have been produced in the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection.

The network denotes any setup which consists of at least two apparatus connected to each other so that information can be transmitted from one apparatus to the other. The apparatus capable of communication through the network may be those which are independent from one another or those which are constituent units of one apparatus.

The term “communication” means wireless and wire communications or a mixture thereof. In the latter case, wireless communication may be carried out in some sections and wire communication may be carried in other sections. Another mode of communication may be such that wire communication is carried out from the first apparatus to the second apparatus and wireless communication is carried out from the second apparatus to the first apparatus.

The above-mentioned information processing apparatus according to the present invention is able to analyze the cellular function relating to the molecules for detection. It is also able to classify the overall relation between molecules, thereby digitizing the relation between the intermolecular interaction and the cellular function.

### BRIEF DESCRIPTION OF THE DRAWINGS

FIG. 1 is a block diagram illustrating a protein analyzing system to which the present invention is applied;

FIG. 2 is a diagram illustrating the classification of the interrelation between two molecules;

FIGS. 3A to 3F are diagrams illustrating the relation between the NN-type molecules and the cellular function;

FIGS. 4A to 4H are diagrams illustrating the relation between the PN-type molecules and the cellular function;

FIGS. 5A to 5H are diagrams illustrating the relation between the PN-type molecules and the cellular function;

FIGS. 6A to 6H are diagrams illustrating the relation between the PN-type molecules and the cellular function;

FIGS. 7A to 7G are diagrams illustrating the relation between the PN-type molecules and the cellular function;

FIGS. 8A to 8G are diagrams illustrating the relation between the PN-type molecules and the cellular function;

FIGS. 9A to 9F are diagrams illustrating the relation between the PN-type molecules and the cellular function;

FIGS. 10A to 10G are diagrams illustrating the relation between the PP-type molecules and the cellular function;

FIGS. 11A to 11G are diagrams illustrating the relation between the PP-type molecules and the cellular function;

FIGS. 12A to 12G are diagrams illustrating the relation between the PP-type molecules and the cellular function;

FIGS. 13A to 13G are diagrams illustrating the relation between the PP-type molecules and the cellular function;

FIGS. 14A to 14F are diagrams illustrating the relation between the PP-type molecules and the cellular function;

FIG. 15 is a diagram illustrating the relation between the NN-type molecules and the cellular function;

FIG. 16 is a diagram illustrating the relation between the PN-type molecules and the cellular function;

FIG. 17 is a diagram illustrating the relation between the PP-type molecules and the cellular function;

FIG. 18 is a diagram illustrating how to calculate the cell score;

FIG. 19 is a diagram illustrating how to calculate the cell score;

FIG. 20 is a diagram illustrating the simulation which is carried out when the prescribed cell score is changed;

FIG. 21 is a diagram illustrating one example of the molecule network;

FIG. 22 is a flow chart illustrating the process for analysis;

FIG. 23 is a flow chart illustrating the process for accumulating points;

FIG. 24 is a flow chart illustrating the process 1 for inferring the target molecule;

FIG. 25 is a flow chart illustrating the process 2 for inferring the target molecule; and

FIG. 26 is a diagram illustrating the structure of a personal computer.

### DETAILED DESCRIPTION OF THE PREFERRED EMBODIMENTS

The following is a detailed description of the embodiments according to the present invention. This description is intended to ensure that the embodiments according to the present invention conform to the specification and drawings therein. The embodiments may include those which have the constituents of the present invention which are not shown in the specification or the drawings therein. This does not necessarily mean that such embodiments do not correspond to the constituents of the present invention. Conversely, even though some embodiments may be written as conforming to the constituents of the present invention, it does not necessarily mean that such embodiments do not conform to other constituents than the constituents.

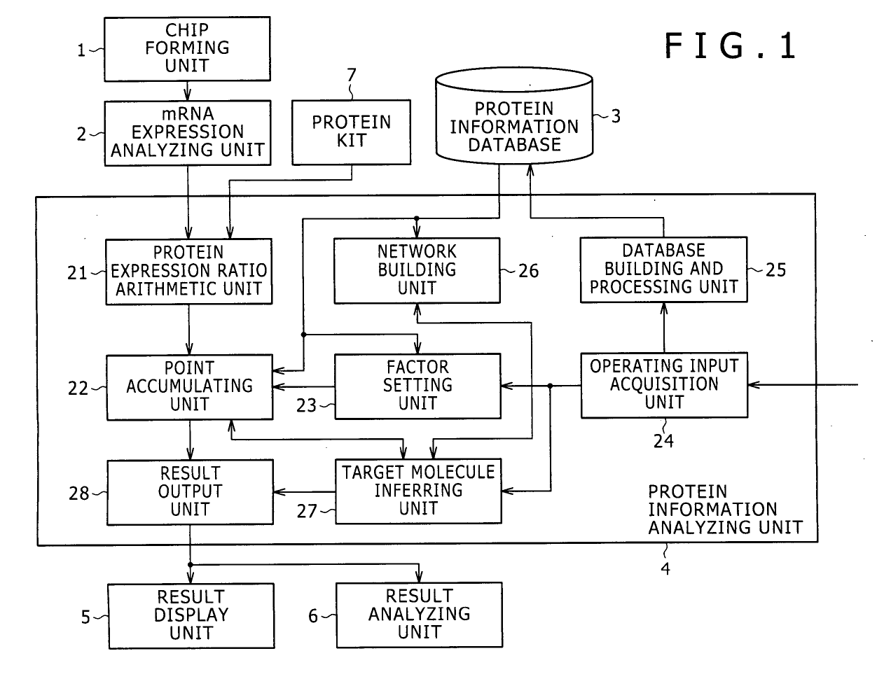

The information processing apparatus according to the present invention has acquisition means (such as the arithmetic unit 21 shown in FIG. 1 which calculates the ratio of the amount of protein expressed) that acquires the amount of the molecules for detection which have been produced by control cells (such as normal cells) and sample cells, arithmetic means (such as the point accumulating unit 22 shown in FIG. 1) that receives from the acquisition means the information about the amount of the molecules for detection which have been produced by the control cells and the sample cells (such as the ratio of protein expressed which is inferred from the amount of mRNA expressed or measured by the protein kit 7), thereby calculating the score that indicates whether one of the two molecules for detection promotes or suppresses the other and also indicates whether the cellular function is promoted or suppressed depending on the combination of cellular functions for the mutual promotion or suppression between the two molecules for detection, and output control means (such as the result output unit 28 shown in FIG. 1) that controls the output of the score which has been calculated by the arithmetic means for the cellular function.

The combination of the two molecules for detection is classified into the following five categories according to the interrelation between the two molecules; the first category (such as NN-type) applicable to two molecules which suppress each other, the second category (such as PN-type) applicable to two molecules the first one of which promotes the second one and the second one of which suppresses the first one, the third category (such as PP-type) applicable to two molecules which promote each other, the fourth category (such as N-type) applicable to two molecules only one of which promotes the other, and the fifth category (such as P-type) applicable to two molecules only one of which suppresses the other.

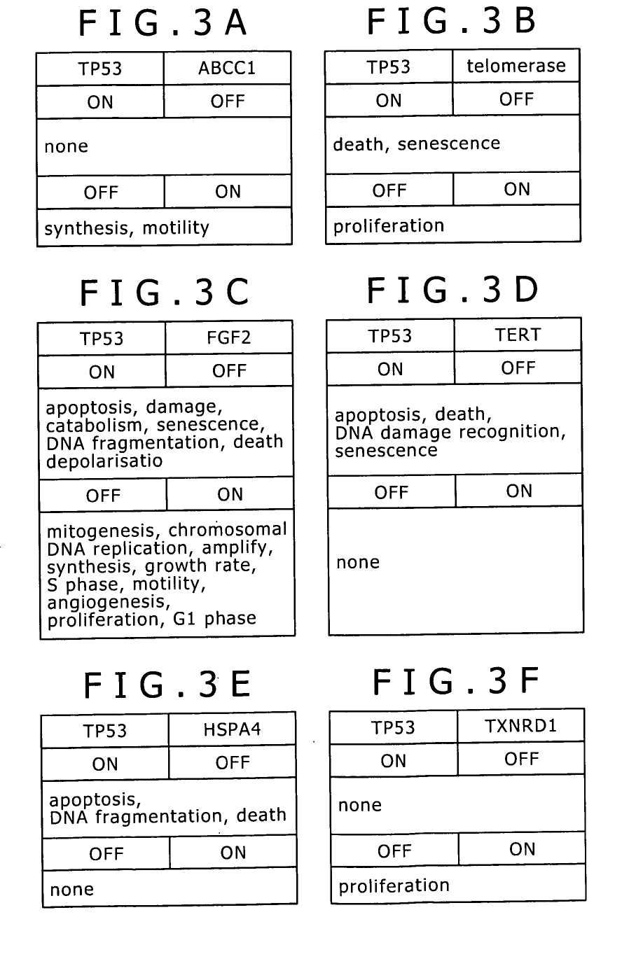

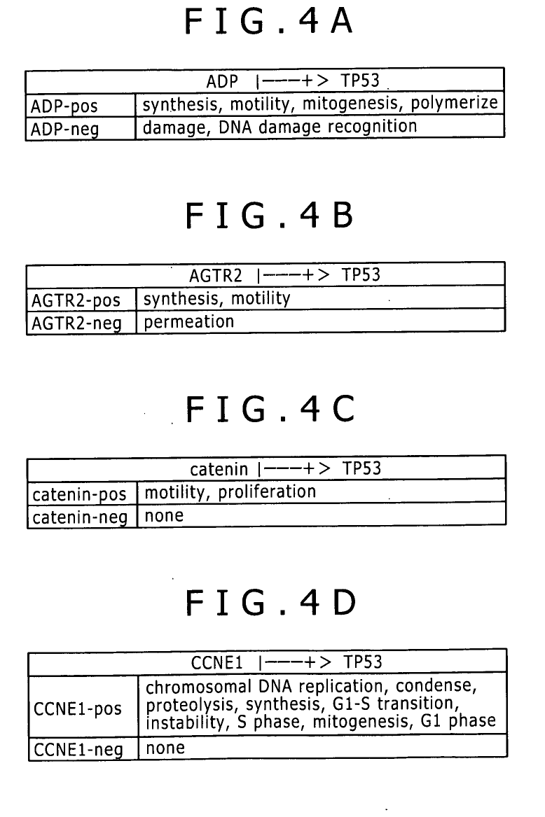

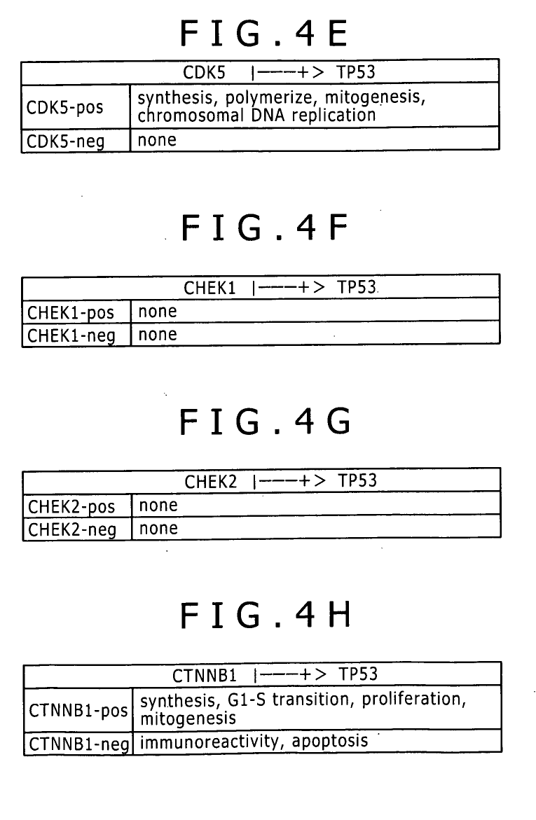

The information processing apparatus may additionally have storage means (such as the protein information database 3) that stores in a table form (shown in FIGS. 3 and 4) the information about the combination of the two molecules for detection which are classified into any of the five categories (as shown in Tables 1 to 7) and the cellular function relating to the mutual promotion or suppression of the two molecules for detection.

The information processing apparatus may additionally have inferring means (such as the target molecule inferring unit 27 shown in FIG. 1) that infers the score for the cellular function when there is any change in the amount of the molecules for detection which have been produced in the control cells and the sample cells after it has been acquired by the acquisition means.

The information processing apparatus may additionally have network building means (such as the network building unit 26 shown in FIG. 1) that builds a network for the information about the interrelation of the molecules for detection, so that the inferring means calculates the effect of change in the amount of the molecules for detection which have been produced on other molecules based on the network which has been built by the network building means, thereby inferring the score for the cellular function.

The information processing apparatus may additionally have analyzing means (such as the result analyzing unit 6 shown in FIG. 1) that analyzes the change with time of the cellular function based on the score for the cellular function, with its output being controlled by the output control means.

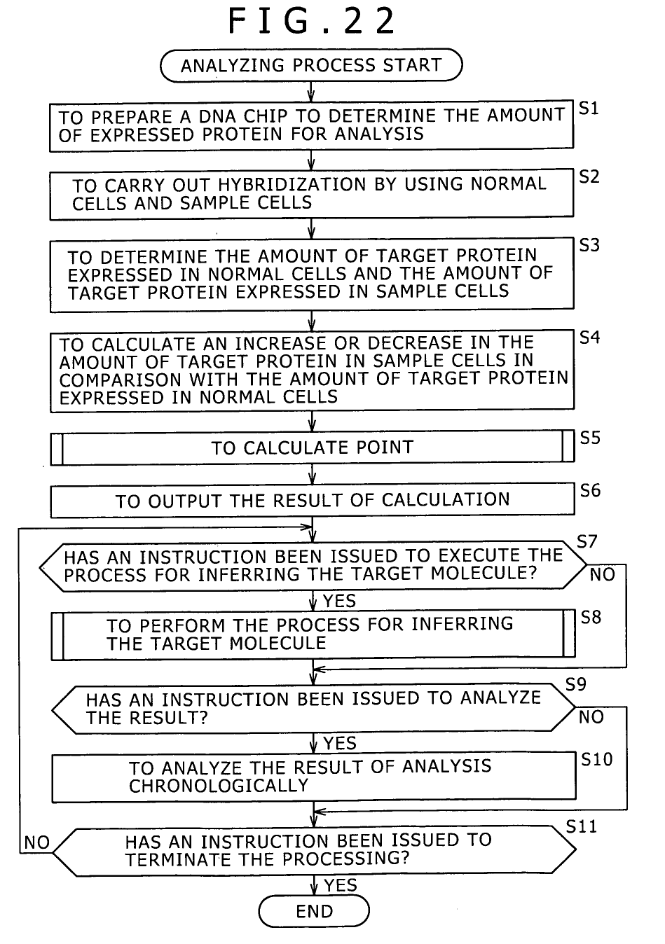

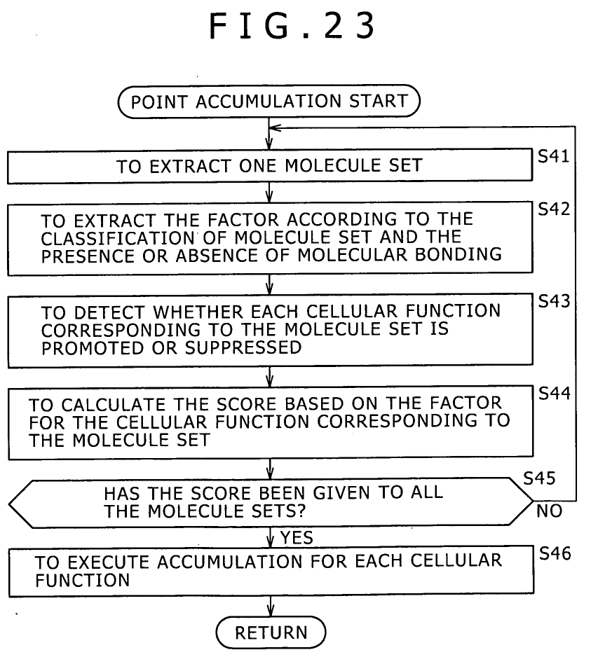

The information processing method or program according to the present invention includes the step of acquiring the amount of the molecules for detection which have been produced in the control cells (such as normal cells) and the sample cells (the step being represented by Step S3 in FIG. 22), and the step of receiving the information about the amount of the molecules for detection which have been produced in the control cells and the sample cells (the information being the ratio of expression of protein), thereby calculating the score that indicates whether one of the two molecules for detection promotes or suppresses the other and also indicates whether the cellular function is promoted or suppressed depending on the combination of cellular functions for the mutual promotion or suppression between the two molecules for detection (the step being represented by Step S5 in FIG. 22 or the process explained with reference to FIG. 23).

The information processing system according to the present invention includes an analyzing unit (such as the mRNA expression analyzing unit 2 shown in FIG. 1) that analyzes the amount of the molecules for detection which have been produced in the control cells (such as normal cells) and the sample cells and an information processing apparatus (such as the protein information analyzing unit 4 shown in FIG. 1) that analyzes the information about the cellular function relating to the mutual promotion or suppression of the two molecules for detection. The information processing apparatus has acquisition means, arithmetic means, and output control means. The acquisition means (such as the arithmetic unit 21 shown in FIG. 1 calculates the ratio of the amount of protein expressed) that acquires the amount of the molecules for detection which have been produced by control cells and sample cells. The arithmetic means (such as the point accumulating unit 22 shown in FIG. 1) receives from the acquisition means the information about the amount of the molecules for detection which have been produced by said control cells and said sample cells (said information being the ratio of protein expressed), thereby calculating the score that indicates whether one of the two molecules for detection promotes or suppresses the other and also indicates whether the cellular function is promoted or suppressed depending on the combination of cellular functions for the mutual promotion or suppression between the two molecules for detection. The output control means (such as the result output unit 28 shown in FIG. 1) controls the output of the score which has been calculated by the arithmetic means for the cellular function.

The embodiment of the present invention will be described with reference the accompanying drawings.

FIG. 1 is a block diagram illustrating the structure of the protein information analyzing system to which the present invention is applied.

The protein information analyzing system includes the chip forming unit 1, the mRNA expression analyzing unit 2, the protein information database 3, the protein information analyzing unit 4, the result display unit 5, and the result analyzing unit 6. It may also have the protein kit 7.

The chip forming unit 1 yields a DNA chip (or DNA microarray) which has as the probe a nucleic acid with the base sequence structure complementary to the molecule (protein) for detection.

The mRNA expression analyzing unit 2 is so designed as to drop the control target and the detection target onto the DNA chip which has been prepared by the chip forming unit 1, thereby determining the amount of the molecule (protein) for detection in each case. The control target is produced by the mRNA collected from the normal cell (control cell), and the detection target is obtained by reverse transcription (for duplication) of the complementary DNA (cDNA) from the mRNA collected from the sample cell. In other words, the mRNA expression analyzing unit 2 performs hybridization, which utilizes the reaction to form the complementary strands (double strands) between nucleic acids each having the complementary base sequence, and then determines, by fluorescence intensity analysis with an intercalator, the amount of the molecule (protein) for detection which has been expressed in the normal cell and the amount of the molecule (protein) for detection which has been expressed in the sample cell, and supplies the thus obtained result to the protein information analyzing unit 4.

The foregoing units may be replaced by the protein kit 7, which is designed to detect comprehensively the molecules (proteins) for detection by using protein chips.

The protein information database 3 stores information about the protein to be used for processing by the protein information analyzing unit 4. The protein information database 3 may be connected, by wire or wireless (e.g., through the Internet or LAN or WAN network), directly to the protein information analyzing unit 4. It may also be installed inside the protein information analyzing unit 4.

The combination of two different protein molecules can be classified into five categories according to their intermolecular interactions. The protein information database 3 stores information about the classification of the combination of two protein molecules belonging to each category. (The classification is referred to as molecule set.)

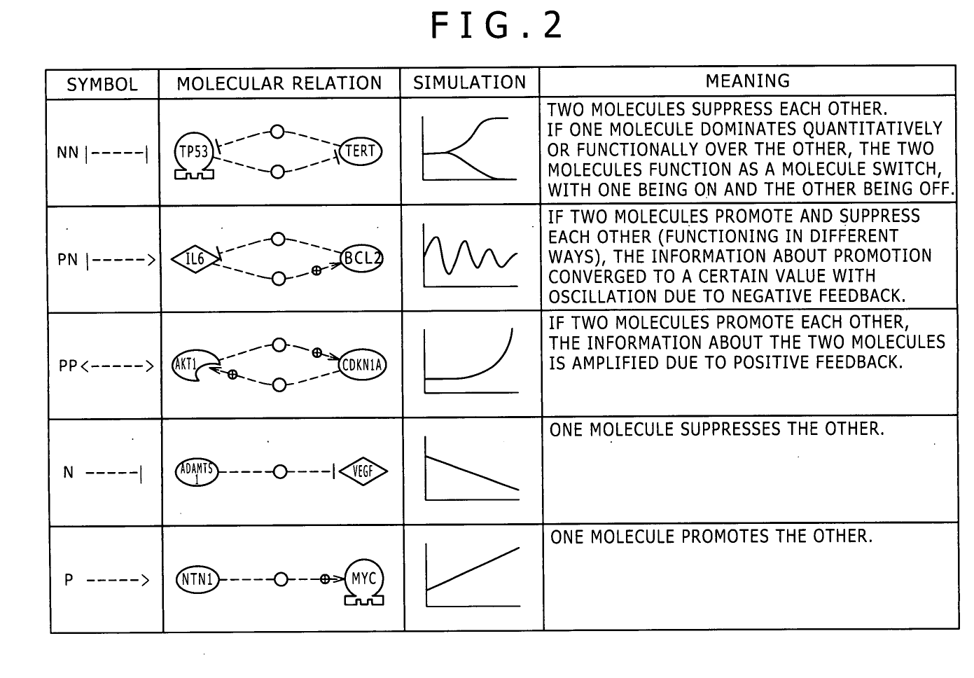

As shown in FIG. 2, the combinations of two molecules (or the intermolecular interrelations between two molecules) are classified into five categories (NN-type, PN-type, PP-type, P-type, and N-type) according to whether one molecule promotes or suppresses the other.

The NN-type denotes a combination in which two molecules suppress each other. The two molecules in the NN-type combination function as the molecular switch, with one representing “ON” if it dominates over the other quantitatively and functionally, and the other representing “OFF”.

The PN-type denotes a combination in which the first molecule promotes the second molecule and the second molecule suppresses the first molecule. In other words, two molecules perform contradictory functions (promotion and suppression) on each other. In this case, the information about the molecule for promotion converges on a certain value with oscillation as the result of negative feedback.

The PP-type denotes a combination in which two molecules promote each other. While two molecules are promoting each other, the information about the two molecules is amplified as the result of positive feedback.

The P-type denotes a combination in which one molecule promotes the other. The N-type denotes a combination in which one molecule suppresses the other.

Tables 1 to 9 show the NN-type combination (molecule set) of molecules.

TABLE 1

| Protein A | | Protein B |
|---|---|---|
| A2N | NN | LRPAP1 |
| A2N | NN | PLG |
| ABO | NN | EGF |
| ACE | NN | ANGPTA |
| ACE | NN | BDK |
| ACE | NN | CIIorf3 |
| ACE | NN | cyclic GNP |
| ACE | NN | HHL |
| ACE | NN | [symbol] E |
| ACE | NN | TACI |
| ACHE | NN | acetylthiocholine |
| ACHE | NN | BCHE |
| ADCY2 | NN | AGTR1 |
| ADCY2 | NN | CHR1 |
| ADCY2 | NN | DRD2 |
| ADCY2 | NN | EDN1 |
| ADCY2 | NN | GR [symbol] 2 |
| ADCY2 | NN | NPPA |
| ADCY2 | NN | P2Y receptor |
| ADCY2 | NN | phospholipase C |
| ADCY2 | NN | RAF1 |
| ADCY2 | NN | SSTR5 |
| ADCY2 | NN | TNFO1 |
| ADRBK1 | NN | GRK5 |
| ADRBK1 | NN | RPS6K |
| AFP | NN | TP53 |
| AGT | NN | ADCY2 |
| AGT | NN | ANG |
| AGT | NN | KCKD3 |
| AGT | NN | prosteglandin- |
| | | endoperoxide synthase |
| AGTR1 | NN | AGTR2 |
| AGTR1 | NN | cAYP |
| AHR | NN | estradiol |
| AKT1 | NN | BAK |
| AKT1 | NN | FOS |
| AKT1 | NN | GRB2 |
| AKT1 | NN | PCK2 |
| AKT1 | NN | PIK2B |
| AKT1 | NN | PTPK6 |
| AKT1 | NN | RAP1GA1 |
| AKT1 | NN | TKFAIP8 |
| ALB | NN | ELA2 |
| ALB | NN | FUT4 |
| ALB | NN | hyaluronoglucuronidase |
| ALB | NN | Na+, K+ ATPase |
| ananain | NN | BDK |
| ANGPT2 | NN | ACK |
| APAF1 | NN | ABL1 |
| APC | NN | CCND1 |
| APC | NN | SNADA |
| APOA1 | NN | cholesteryl ester |
| APOE | NN | LRPAP1 |
| AOP2 | NN | AOP3 |
| ARF6 | NN | TFAP2A |
| AVP | NN | CRHP1 |
| AVP | NN | REN |
| BAD | NN | BAK1 |
| BAX | NN | BCL2 |
| Bbaa1l | NN | SELE |
| BCAR1 | NN | GNB2L1 |
| BCAR1 | NN | NEDD9 |
| BCL2 | NN | anyloid protein |
| BCL2 | NN | BAK1 |
| BCL2 | NN | HRK |
| BCL2 | NN | NR3C1 |
| BCL2 | NN | PDCD3 |
| BCL2 | NN | TNFAIP8 |
| BCL2 | NN | TNFRSF6 |

TABLE 2

| Protein A | | Protein B |
|---|---|---|
| BCL2L1 | NN | BAK1 |
| BCL2L1 | NN | BID |
| BCL2L1 | NN | sphingosine |
| BCL2L1 | NN | TNFRASF6 |
| BCL6 | NN | PRDH1 |
| BDK | NN | ANP |
| BDK | NN | DNOLI |
| BDK | NN | NNE |
| BDK | NN | TGFA |
| beta-o-glucose | NN | CAT |
| oxidase | | |
| BF | NN | SERPING1 |
| BH2HB2 | NN | BHLHB3 |
| BIO | NN | TRFAIP8 |
| BIRC3 | NN | CASP3 |
| BIRC3 | NN | TNFSF10 |
| BHP4 | NN | BRP4 receptor |
| BHP4 | NN | PITX2 |
| BHP4 | NN | tumor necrosis factor |
| BU618 | NN | CDC2 |
| CALCA | NN | calmodulin |
| calmodulin | NN | ADREX1 |
| calmodulin | NN | ELK1 |
| calmodulin | NN | GAP43 |
| calmodulin | NN | SHAD2 |
| calpain | NN | APOE |
| calpain | NN | NEXBIA |
| CALR | NN | calcitriol |
| casein kinase | NN | GJA1 |
| CASP3 | NN | BIRC2 |
| CASP3 | NN | CD28 |
| CASP3 | NN | CDC42 |
| CASP3 | NN | COLI1A1 |
| CASP3 | NN | dihydrosphingosine |
| | | kinase |
| CASP3 | NN | HSPB2 |
| CASP3 | NN | IGF2 |
| CASP3 | NN | map kinase |
| CASP3 | NN | NCL1 |
| CAT | NN | CASP3 |
| CAT | NN | cytokine activity |
| CAT | NN | HIFIA |
| CAT | NN | HNOK1 |
| CAT | NN | LP0 |
| CAT | NN | HAPKAP1 |
| catecholamine | NN | mitochondrial |
| | | processing peptidase |
| CAV1 | NN | ADCY2 |
| CAV1 | NN | FGF2 |
| CAV1 | NN | HDL |
| CBL | NN | TCN2 |
| CCL2 | NN | GJA1 |
| CCL2 | NN | RGS1 |
| CCL2 | NN | RGS3 |
| CCL2 | NN | RGS4 |
| CCL42 | NN | CYP2E1 |
| CCNNA2 | NN | CDKN2A |
| CCNA2 | NN | RBL2 |
| CCND1 | NN | CDKN2A |
| CCND1 | NN | GSK3B |
| CCND1 | NN | PPARA |
| CCND1 | NN | PPARG |
| CCNE1 | NN | STAT3 |
| CCNE1 | NN | RBI |
| CCNE1 | NN | SNARCA4 |
| CCR5 | NN | coreceptor |
| CD4 | NN | PRF1 |
| CDB6 | NN | CD28 |
| CDA | NN | SLC9A3 |
| CCDC2 | NN | CDKNIA |
| CDC2 | NN | NYT1 |
| CDC2 | NN | PAPOLA |
| CDC2 | NN | RB1 |
| CDC2 | NN | NEE1 |
| CDC42 | NN | GD1 |
| CDC42 | NN | NLC1 |
| CDH1 | NN | CDC2 |
| CDH1 | NN | CDC20 |

TABLE 3

| Protein A | | Protein B |
|---|---|---|
| CDK2 | NN | CDK2AP1 |
| CDK2 | NN | CIB1 |
| CDK2 | NN | PSND9 |
| CDK2 | NN | SNARCA4 |
| CDK4 | NN | HYOD1 |
| CDK5 | NN | CDC2 |
| CDK5 | NN | CDKN2A |
| CDK6 | NN | CDKN1A |
| CDKN1A | NN | E2F4 |
| CDKN1A | NN | GSK3B |
| CDKN1B | NN | CHX10 |
| CDKN1B | NN | CISH |
| CDKN1B | NN | IL3 |
| CDKN1B | NN | RHOA |
| CDKN1B | NN | SOCS3 |
| CDKN1B | NN | transcription factor |
| CDKN2A | NN | CDKN26 |
| CDKN2B | NN | RBI |
| CEBPB | NN | cANP |
| CEBPB | NN | CTNNB1 |
| CEBPG | NN | tumor necrosis factor |
| chloroxazone | NN | CYP2E1 |
| cholesterol | NN | CAV1 |
| cholesterol | NN | PPARA |
| choline-phosphate | NN | RIPK2 |
| cytidylyltransferase | | |
| CISH | NN | SHOC2 |
| CNTF | NN | ADCYAP1 |
| CREB1 | NN | CALCA |
| CREB1 | NN | CREBBP |
| CREB1 | NN | GSK3B |
| CREB1 | NN | NAPK11 |
| CREB1 | NN | protein phosphate 1 |
| CREB1 | NN | PTPN1 |
| CSF1 | NN | INPP50 |
| CSF2 | NN | CCR5 |
| CTNNA1 | NN | ITGB1 |
| CTSS | NN | CST3 |
| CYCS | NN | ABL1 |
| CYCS | NN | DAP13 |
| CYCS | NN | POR |
| CYP1A1 | NN | estrogens |
| CYP2D6 | NN | Cyp3a11 |
| CYP2E1 | NN | CYP1A1 |
| CYP3A4 | NN | CYP3A5 |
| CYP3A4 | NN | NET |
| DAP | NN | ODC1 |
| DAPK1 | NN | integrin |
| deacetylase | NN | HOAC1 |
| DFFB | NN | UTP |
| DIABLO | NN | BIRC4 |
| DUSPI | NN | RAS small monomeric |
| | | GTPase |
| E2FI | NN | PRB2 |
| E2FI | NN | SERPINE1 |
| E3 | NN | DIABLO |
| EBP | NN | tumor necrosis factor |
| EDN1 | NN | BDK |
| EDN1 | NN | cANP |
| EDN1 | NN | estradiol |
| EDN1 | NN | estrosen |
| EDN1 | NN | LPL |
| EDN1 | NN | progesterone |
| EDNRA | NN | AGTR1 |
| EGF | NN | ASCL1 |
| EGF | NN | CLU |
| EGF | NN | CTSB |
| EGF | NN | GCG |
| EGF | NN | IGFBP2 |
| EGF | NN | HYE |
| EGF | NN | TGFB2 |
| EGFR | NN | ANH |
| EGFR | NN | LRTG1 |
| EGFR | NN | PAKCA |
| EGFR | NN | SLC29A1 |
| EGLN3 | NN | HIF1A |

TABLE 4

| Protein A | | Protein B |
|---|---|---|
| EGR1 | NN | SP3 |
| ELA2 | NN | ELN |
| ELA2 | NN | SERPINA3 |
| ELA2 | NN | TINP1 |
| EPH44 | NN | WAP2K4 |
| EPX | NN | H2O2 |
| ERBB2 | NN | CAV1 |
| ERBB2 | NN | progesterone |
| ERBB2 | NN | RHOB |
| ERK activator | NN | PPARA |
| kinase | | |
| F10 | NN | protein C (activated) |
| F2 | NN | PIP2 |
| FGF1 | NN | FIBP |
| FGF2 | NN | GSK3B |
| FGF2 | NN | HPSE |
| FGF2 | NN | NFXBIA |
| FGF2 | KH | THBS1 |
| FN1 | NN | ACTA2 |
| FN1 | NN | CDC42 |
| FN1 | NN | PLAU |
| FOS | NN | CEBPA |
| FOS | NN | KLK3 |
| FOS | NN | Ha+/K+ ATPase |
| FOXO1A | NN | PPARG |
| FXN | NN | INHBA |
| FYN | NN | DUSP1 |
| GATA1 | NN | SPI1 |
| GCG | NN | DPP4 |
| GCG | NN | ENTPD2 |
| GH1 | NN | ITIH4 |
| GHA0 | NN | Phosphatidylinositol |
| | | 3-Kinase |
| GNB2L1 | NN | CTNNA2 |
| GNRH1 | NN | HR3C1 |
| GNRH1 | NN | SST |
| GPC3 | NN | IGF2 |
| GPI | NN | IGFBP3 |
| GRB2 | NN | PDGFRB |
| GRB2 | NN | PSKD9 |
| GRPR | NN | BRS3 |
| GRPR | NN | NEBR |
| GSK3B | NN | CDKN1B |
| GSK3B | NN | IRS2 |
| GSK3B | NN | RPS6KB1 |
| GSK3B | NN | Nnt |
| GSTN1 | NN | VAP3K5 |
| GTP | NN | CALCA |
| GTP | NN | POFK1 |
| HAND2 | NN | IL13 |
| HDAC1 | NN | HDAC2 |
| HDAC4 | NN | HEF2A |
| HDL | NN | LPL |
| HES1 | NN | NEUROG3 |
| HGF | NN | interleukin IL12 |
| HGF | NN | THBS1 |
| histone deacetylase | NN | CFTR |
| histone deacetylase | NN | E2F4 |
| HVGA2 | NN | CCL2 |
| HRF4A | NN | NO |
| HOXB1 | NN | EGR2 |
| HRAS | NN | RHOB |
| HSPA4 | NN | caspase |
| HSPA4 | NN | PKC |
| HSPB1 | NN | DAKX |
| ICAN1 | NN | SELL |
| ICAN1 | NN | TNFRSFG |
| IFNG | NN | cytochrome P450 |
| IFNG | NN | EP300 |
| IFHG | NN | IL10 |
| IFHG | NN | IL13 |
| IFHG | NN | IL17 |
| IFNG | NN | IL5 |
| IFNG | NN | PPARG |
| IGF1 | NN | ADCY2 |
| IGF1 | NN | GDG |
| IGF1 | NN | HSPCA |
| IGF1 | NN | ILIF8 |
| IGF1 | NN | LIF |
| IGF1 | NN | turor necrosis factor |

TABLE 5

| Protein A | | Protein B |
|---|---|---|
| IGF2 | NN | H19 |
| IGFALS | NN | IGF2 |
| IGFBP1 | NN | IGF2 |
| IGFBP1 | NN | IGFBP2 |
| IGFBP3 | NN | chorionic gonadotropin |
| IL1 | NN | NR3C1 |
| IL10 | NN | lectin |
| IL10 | NN | NNP9 |
| IL13 | NN | interleukin IL13 |
| | | receptor |
| IL18 | NN | CASP3 |
| IL18 | NN | IL1R1 |
| IL18BP | NN | IL18 |
| IL1A | NN | estradiol |
| IL1A | NN | PRKCA |
| IL1A | NN | thyroid stimulating |
| | | hormone |
| IL1B | NN | CCL21 |
| IL1B | NN | cytochrome P450 |
| IL1B | NN | EP0 |
| IL1B | NN | GHRL |
| IL1B | NN | HNF4A |
| IL1B | NN | NFKB1B |
| IL1B | NN | SDC1 |
| IL1B | NN | SPARC |
| IL1B | NN | TIVP3 |
| IL1F8 | NN | ELK |
| IL1F8 | NN | NFKBIA |
| IL1F8 | NN | SP3 |
| IL1R1 | NN | PTGS2 |
| IL1R1 | NN | tumor necrosis factor |
| IL2 | NN | ELA2 |
| IL2 | NN | PAX5 |
| IL2 | NN | TGFB2 |
| IL4 | NN | ALOX5 |
| IL4 | NN | B7H3 |
| IL4 | NN | CXCL9 |
| IL4 | NN | FGF2 |
| IL4 | NN | IFNA1 |
| IL4 | NN | TNPO1 |
| IL4 | NN | TRERF1 |
| IL4 | NN | VIP |
| IL5RA | NN | IL5 |
| IL6 | NN | APOE |
| IL6 | NN | CDKH1A |
| IL6 | NN | CYP1A2 |
| IL6 | NN | cytochrome P450 |
| IL6 | NN | GFI1B |
| IL6 | NN | NTP |
| IL6 | NN | NYB |
| IL6 | NN | PPARG |
| IL6 | NN | RB1 |
| IL6 | NN | SELL |
| IL6 | NN | vitamin D |
| INS | NN | alpha2 adrenoceptor |
| INS | NN | ARRB1 |
| INS | NN | cAMP-dependent protein |
| | | kinase, catalyst |
| INS | NN | CYP2E1 |
| INS | NN | DPP4 |
| INS | NN | epinephrine |
| INS | NN | GAL |
| INS | NN | glycogen synthase |
| | | kinase 3 |
| INS | NN | IFNG |
| INS | NN | LDL |
| INS | NN | PFKFB1 |
| INS | NN | phosphoenolpyruvate |
| | | carboxykinase |
| INS | NN | PLTP |
| INS | NN | protein tyrosine |
| | | phosphatase |
| INS | NN | PTGIS |
| INS | NN | Rho kinase |
| INS | NN | RHOA |
| INS | NN | TERF2IP |
| INS | NN | tumor necrosis factor |
| INS | NN | VLDL |

TABLE 6

| Protein A | | Protein B |
|---|---|---|
| interleukin IL12 | NN | IL5 |
| IRAN4 | NN | TLR4 |
| IRF4 | NN | BCL6 |
| IRF4 | NN | IFNA1 |
| IRS2 | NN | TRS4 |
| ITIH4 | NN | OCR5 |
| ITK | NN | CDH1 |
| JAK1 | NN | PTPN6 |
| JAK2 | NN | PTPN6 |
| JAK2 | NN | SOCS3 |
| JAK2 | NN | TYRP1 |
| JUN | NN | AR |
| JUN | NN | CDK2 |
| JUN | NN | CEBPA |
| JUN | NN | CTLA4 |
| JUN | NN | Na+/K+ ATPase |
| JUN | NN | NR3C1 |
| KIT | NN | PTPN6 |
| LCP1 | NN | ELA2 |
| LCP1 | NN | SERPING1 |
| LCP1 | NN | TFP1 |
| LEP | NN | CDH1 |
| LEP | NN | GAL |
| LEP | NN | GCG |
| LEP | NN | RB1 |
| LEP | NN | serotonin |
| LEP | NN | SST |
| ligase | NN | EGFR |
| LOX | NN | HRAS |
| LPA | NN | cAMP |
| LPA | NN | DGPP phosphatase |
| LPL | NN | LIPC |
| LPL | NN | phosphatidylcholine |
| LPL | NN | TG |
| LYZ | NN | HIST1H4D |
| NADO | NN | TNFRSFIA |
| nap kinase | NN | ADRBK1 |
| nap kinase | NN | NYOD1 |
| nap kinase | NN | PTEN |
| NAP3K1 | NN | NAPK3IPI |
| NAPK1 | NN | BRF1 |
| NAPK1 | NN | CAV1 |
| NAPK1 | NN | CDC2 |
| NAPK1 | NN | DUSP2 |
| NAPK1 | NN | NOS3 |
| NAPK1 | NN | pathway-specific SNAD |
| | | protein |
| NAPK1 | NN | PTPN6 |
| NAPK1 | NN | RAP16A1 |
| NAPK10 | NN | CDK5 |
| NAPK14 | NN | IT6A4 |
| NAPK14 | NN | Phosphatidyl inositol |
| | | 3-kinase |
| NAPK14 | NN | TXN |
| NAPK14 | NN | XDH |
| NAPK3 | NN | EPHB2 |
| NAPK3 | NN | protein phosphatase 2A |
| NAPK8 | NN | CAV1 |
| NAPK8 | NN | IL4 |
| NAPK8 | NN | NPPA |
| NAPK8 | NN | PPARA |
| NAPK8 | NN | protein tyrosine |
| | | phosphatase |
| NAPK8 | NN | PSEN1 |
| NAPK8 | NN | RARA |
| matrix | NN | HYP9 |
| metalloproteinase | | |
| NHC2TA | NN | IL10 |
| NNP2 | NN | THBS2 |
| NNP9 | NN | gelatin |
| HYP9 | NN | TIYP3 |
| NP0 | NN | LTF |
| NSYB | NN | LTA |
| HTBP | NN | EP300 |
| NUC2 | NN | PTGS2 |

TABLE 7

| Protein A | | Protein B |
|---|---|---|
| NYC | NN | BCL2 |
| NYC | NN | CAV1 |
| NYC | NN | BDKN1A |
| NYC | NN | CDKN2B |
| NYC | NN | CE8PA |
| NYC | NN | EP300 |
| NYC | NN | IFNA1 |
| NYC | NN | INHBA |
| NYC | NN | NYOD1 |
| NYC | NN | pathway-specific SNAD |
| | | protein |
| NYC | NN | ZBTB16 |
| NYOD1 | NN | RAS small monomeric |
| | | GTPase |
| NGFB | NN | CDK2 |
| NGFB | NN | EPAS1 |
| NGFB | NN | NO |
| NGFB | NN | STAT3 |
| norepinephrine | NN | LEP |
| NOS2A | NN | ARG1 |
| NOS2A | NN | GAPH1 |
| NOS2A | NN | HNOX1 |
| NOS2A | NN | RHOA |
| NPPA | NN | ANP32A |
| NPPA | NN | D9Ngc42e |
| NPPA | NN | PRKG1 |
| NPY | NN | Ca-ATPase |
| NPY | NN | GHRH |
| NR3C1 | NN | NFIC |
| NR3C1 | NN | Nuclear factor NF |
| | | kappa B |
| NTRK1 | NN | NGFR |
| PARP1 | NN | CASP6 |
| PARP1 | NN | cytokine activity |
| pathway-specific | NN | EP300 |
| SNAD protein | | |
| PANR | NN | BCL2 |
| PAK5 | NN | SPI1 |
| PDPK1 | NN | PPP1R13B |
| PGR | NN | Nuclear factor NF |
| | | kappa B |
| PGR | NN | RELA |
| Phosphatidylinositol | NN | CASP3 |
| 3-kinase | | |
| Phosphatidylinositol | NN | CASP9 |
| 3-kinase | | |
| PIK3CA | NN | PTEN |
| PKA | NN | CXCL12 |
| PLA2G18 | NN | HTATIP |
| PLA2G18 | NN | phosphatidylcholine |
| PLG | NN | SERPINB5 |
| PLG | NN | SERPINF2 |
| PNA | NN | CDK2 |
| PONC | NN | ascorbic acid |
| PONC | NN | doparine D2 receptor |
| PPARA | NN | Nuclear factor NF |
| | | kappa B |
| PPARA | NN | STAT58 |
| PPARA | NN | tumor necrosis factor |
| PPARG | NN | KLF2 |
| PPARG | NN | Nuclear factor NF |
| | | kappa B |
| PPARG | NN | STAT58 |
| PRDH1 | NN | PAX5 |
| PRKCA | NN | DGKZ |
| PRKCA | NN | protein phosphatase 2A |
| PRKCA | NN | PTHLH |
| PRKCA | NN | TNFRSF6 |
| PRL | NN | ANXA5 |
| PRL | NN | DHT |
| protein phosphatase | NN | EGFR |
| protein phosphatase 1 | NN | RPS6KB1 |
| protein phosphatase 1 | NN | SYK |
| protein tyrosine | NN | PRKCD |
| phosphatase | | |
| PRV1 | NN | ubiquitin |
| PSEN1 | NN | PSEN2 |
| PSEN1 | NN | SAP kinase |

TABLE 8

| Protein A | | Protein B |
|---|---|---|
| PSND9 | NN | CDK6 |
| PSND9 | NN | RAS small monomeric |
| | | GTPase |
| PTEN | NN | BCAR1 |
| PTEN | NN | CREB1 |
| PTEN | NN | Nuclear factor NF |
| | | kappa B |
| PTEN | NN | RPS6K |
| PTEN | NN | TNFRSF6 |
| PTEN | NN | tumor necrosis factor |
| PTGS2 | NN | cANP |
| PTGS2 | NN | GSK3B |
| PTGS2 | NN | NUC5AC |
| PTGS2 | NN | PPARG |
| PTH | NN | TNFRSFI1B |
| PTHLH | NN | BHLHB2 |
| PTK2B | NN | BCL2L1 |
| PTK2B | NN | CHRN1 |
| PTPN6 | NN | receptor signaling |
| | | protein |
| RAC1 | NN | EFNA1 |
| RAC1 | NN | GDI |
| RAC1 | NN | GNA12 |
| RAC1 | NN | PTK2 |
| RAC1 | NN | RAC2 |
| RAF1 | NN | CAV1 |
| RAF1 | NN | GAP |
| RAF1 | NN | NO |
| RAF1 | NN | RASGRF1 |
| RARA | NN | CREBBP |
| RARA | NN | UBE1L |
| RARA | NN | VDR |
| RB1 | NN | BRCA1 |
| RB1 | NN | COKN2A |
| RB1 | NN | INHBA |
| RB1 | NN | NDY2 |
| RBL1 | NN | E2F1 |
| RBL2 | NN | E2F1 |
| REN | NN | ACE |
| REN | NN | RENBP |
| RGS4 | NN | NOS3 |
| RHOA | NN | cyclic GNP |
| RHOA | NN | myosin phosphatase |
| RHOA | NN | PKA |
| RPS6KB1 | NN | PIK3RI |
| SCT | NN | CALCA |
| SELL | NN | CD44 |
| SERPINA1 | NN | ELA2 |
| SERPINE1 | NN | protein C (activated) |
| SERPINF2 | NN | PLAUR |
| SKIL | NN | SNAD2 |
| SLC9A3 | NN | SLC9A1 |
| SNAD3 | NN | NYOD1 |
| SOD2 | NN | CAT |
| SOD2 | NN | PGE |
| SPARC | NN | FGF2 |
| SPI1 | NN | GATA2 |
| SRC | NN | CSK |
| src family | NN | LCK |
| src family | NN | CSK |
| SST | NN | ADCY2 |
| SST | NN | IAPP |
| SST | NN | ILIA |
| SST | NN | PPY |
| SST | NN | TRH |
| STAT3 | NN | INHBA |
| STAT5A | NN | CDKN1B |
| STAT5A | NN | ESR2 |
| STAT5A | NN | SOCS3 |
| STAT6 | NN | Nuclear factor NF |
| | | kappa B |
| sterol | NN | LDLR |
| superoxide | NN | NOS3 |
| dismutase | | |
| SYK | NN | fibrinogen |
| TAC1 | NN | noradrenaline |
| TAC1 | NN | NPY |
| TAC1 | NN | SST |
| TEK | NN | ANGPT2 |
| TERF2IP | NN | GAP |

TABLE 9

| Protein A | | Protein B |
|---|---|---|
| TGFB1 | NN | BF |
| TGFB1 | NN | EGPT |
| TGFB1 | NN | CA1 |
| TGFB1 | NN | CCHA2 |
| TGFB1 | NN | DCN |
| TGFB1 | NN | ENTP02 |
| TGFB1 | NN | ESR1 |
| TGFB1 | NN | FOXG18 |
| TGFB1 | NN | GFPT1 |
| TGFB1 | NN | HGF |
| TGFB1 | NN | IGFBP1 |
| TGFB1 | NN | KITL6 |
| TGFB1 | NN | HYC |
| TGFB1 | NN | nitric oxide synthase |
| TGFB1 | NN | NOS2A |
| TGFB1 | NN | PAX8 |
| TGFB1 | NN | PGF |
| TGFB1 | NN | PRL |
| TGFB1 | NN | RBL1 |
| TGFB1 | NN | RELA |
| TGFB1 | NN | TIE |
| TGFB1 | NN | TTF2 |
| thioredoxin | NN | TXN |
| reductase (nadph) | | |
| T [symbol] P1 | NN | S100A4 |
| T [symbol] P2 | NN | GHP3 |
| TNF | NN | ABCC2 |
| TNF | NN | ACDC |
| TNF | NN | ADCY2 |
| TNF | NN | ALB |
| TNF | NN | ANBP |
| TNF | NN | CYP11A1 |
| TNF | NN | CYP17A1 |
| TNF | NN | EDNRA |
| TNF | NN | FLT1 |
| TNF | NN | GATA3 |
| TNF | NN | insulin receptor |
| TNF | NN | TRAK1 |
| TNF | NN | NFXBIB |
| TNF | NN | PPARG |
| TNF | NN | PROS1 |
| TNF | NN | protein phosphatase 1 |
| TNF | NN | REN |
| TNF | NN | SFTPC |
| TNF | NN | THBS1 |
| TNFRSF1A | NN | CCL4 |
| TNFRSF6 | NN | CXCL9 |
| TNFRSF7 | NN | TL10 |
| TP53 | NN | ABOC1 |
| TP53 | NN | BIRC3 |
| TP53 | NN | BRCA2 |
| TP53 | NN | CAK complex |
| TP53 | NN | CDKN1B |
| TP53 | NN | DAXX |
| TP53 | NN | FGF2 |
| TP53 | NN | HSPA4 |
| TP53 | NN | NAP4 |
| TP53 | NN | NR3C1 |
| TP53 | NN | PSEN1 |
| TP53 | NN | RAD51 |
| TP53 | NN | telonerase |
| TP53 | NN | TERT |
| TP53 | NN | TXNRD1 |
| turor necrosis | NN | IL5 |
| factor | NN | NFKBIA |
| ubiquitin | | |
| UTP | NN | ATPase |
| VDR | NN | PWA |
| VEGF | NN | TNFSF15 |
| VIP | NN | DAP |
| VIP | NN | NPY |
| WT1 | NN | EGFR |
| WT1 | NN | PVA |
| YY1 | NN | SREBF1 |
| ZFPN1 | NN | GATA3 |

Tables 10 to 35 show the PN-type combination (molecule set) of molecules.

TABLE 10

| Protein A | | Protein B |
|---|---|---|
| 14-3-3 | PN | RAF1 |
| 1-phosphetioyl inositol- | PN | HSNB |
| 4-phosphate 5-kinase | | |
| 5-HT2 receptor | PN | DRD2 |
| ABCB1 | PN | IL2 |
| ABL1 | PN | BCR |
| ABL1 | PN | HRAS |
| ABL1 | PN | IL7 |
| ABL1 | PN | MAP3K1 |
| ABL1 | PN | MAPK8 |
| ABL1 | PN | NYBBP1A |
| ABL1 | PN | NYC |
| ABL1 | PN | protein tyrosine kinase |
| ABL1 | PN | PTPN1 |
| ABL1 | PN | STAT1 |
| ABL1 | PN | STAT5A |
| ABL1 | PN | transcription factor |
| ACE | PN | EDN1 |
| ACE | PN | EGR1 |
| ACE | PN | TGFB1 |
| ACE | PN | TNF |
| acid phosphatase | PN | KLK3 |
| activin | PN | SNAD2 |
| ACTN1 | PN | ABL1 |
| ACVR1 | PN | SYAD3 |
| ADANTSL1 | PN | FN1 |
| ADCYAP1 | PN | JUN |
| ADD1 | PN | LEP |
| ADD1 | PN | SREBF1 |
| ADP | PN | GCG |
| ADP | PN | HSPA4 |
| ADP | PN | HSPCA |
| ADP | PN | IL18 |
| ADP | PN | TP53 |
| ADRB3 | PN | INS |
| ADRBK1 | PN | NAPK3 |
| AFP | PN | ALB |
| AGRN | PN | CDC42 |
| AGT | PN | ARG2 |
| AGT | PN | HHOX1 |
| AGT | PN | HTATIP |
| AGT | PN | LEP |
| AGT | PN | HAPK9 |
| AGT | PN | PLA2G1B |
| AGT | PN | PPARG |
| AGT | PN | PTGIS |
| AGT | PN | PTH |
| AGT | PN | PTHLH |
| AGT | PN | PTK2 |
| AGT | PN | PTPNI1 |
| AGT | PN | RHOA |
| AGT | PN | SERP1NE1 |
| AGT | PN | SRC |
| AGTR1 | PN | RHOA |
| AGTR2 | PN | NGF8 |
| AGTR2 | PN | IP53 |
| AHR | PN | CYP1A1 |
| AKT1 | PN | BCL2 |
| AKT1 | PN | caspase |
| AKT1 | PN | ERK activator kinase |
| AKT1 | PN | fatty acids |
| AKT1 | PN | FOXD1 |
| AKT1 | PN | IGFBP5 |
| AKT1 | PN | HAP2K2 |
| AKT1 | PN | HR3C1 |
| AKT1 | PN | oxygen |
| AKT1 | PN | PIK3R1 |
| AKT1 | PN | protein phosphatase 2A |
| AKT1 | PN | RHC8 |
| AKT1 | PN | TNFSF6 |
| ALB | PN | FN1 |
| ALB | PN | TL6 |
| ALB | PN | PTGFS |
| ALB | PN | TGFB1 |
| aldosterone | PN | ADH |
| alphaVbeta6 | PN | NNF9 |

TABLE 11

| Protein A | | Protein B |
|---|---|---|
| AVH | PN | IFNG |
| aminopeptidase | PN | BDK |
| ANGPT2 | PN | BDK |
| ANGPT4 | PN | BDK |
| APC | PN | HYC |
| APEK1 | PN | JUN |
| APOE | PN | APP |
| APP | PN | IGF1R |
| APP | PN | PTGS2 |
| APPBP1 | PN | TP53BP2 |
| AR | PN | BCL2 |
| AR | PN | KLK3 |
| arachidonic acid | PN | CAT |
| arachidonic acid | PN | CYP2E1 |
| arachidonic acid | PN | EGFR |
| arachidonic acid | PN | GAP43 |
| ARG2 | PN | AGTR1 |
| ARHGEF11 | PN | PTK2 |
| ARHGEF7 | PN | CDC42 |
| ARHGEF7 | PN | PAK1 |
| ATF1 | PN | CREB1 |
| ATF1 | PN | TGFB1 |
| ATPase | PN | MAPK6 |
| ATR | PN | CHEK1 |
| AVP | PN | AKT1 |
| BAD | PN | AKT1 |
| BAD | PN | PKA |
| BAG1 | PN | HSPA4 |
| BAX | PN | BCL2L1 |
| BCAR1 | PN | RAC1 |
| B-cell receptor | PN | PLCG1 |
| BCL2 | PN | IFNG |
| BCL2 | PN | YDN2 |
| BCL2 | PN | RAS small monomeric |
| | | GTPase |
| BCL2 | PN | RU-486 |
| BCL2L1 | PN | DSIPI |
| BCL2L1 | PN | MAPK1 |
| BCL2L1 | PN | HFKBIA |
| BCL6 | PN | TKFRSF5 |
| BDK | PN | ADH |
| BDK | PN | RAC1 |
| BDK | PN | RHOA |
| BDK | PN | SELP |
| BDNF | PN | NAPK3 |
| beta adrenoceptor | PN | GCG |
| BGN | PN | RAC1 |
| BGN | PN | RHOA |
| BID | PN | BAX |
| BikIk | PN | BCL2L1 |
| BIRC4 | PN | PRSS25 |
| BIRC4 | PN | TGFB1 |
| BIRC5 | PN | AGT |
| BIRC7 | PN | DIABLO |
| blood coagulation | PN | IL6 |
| factor XII | | |
| BYP15 | PN | KITLG |
| BYP2 | PN | BYP4 |
| BYP2 | PN | SYAD4 |
| BYP2 | PN | TGFB1 |
| BYP4 | PN | CASP3 |
| BYP4 | PN | CASP8 |
| BRCA1 | PN | CASP3 |
| BRCA1 | PN | HAPK3 |
| C11orf3 | PN | BDK |
| CAK complex | PN | CCNA2 |
| CAK complex | PN | CDK4 |
| CALCA | PN | YAPK1 |
| CALCA | PN | YAPK3 |
| CALCA | PN | POYC |
| calcineurin | PN | ELK1 |
| calcineurin | PN | NAPK8 |
| calcineurin | PN | NEF2A |
| calcium | PN | PTGIS |
| calcium | PN | TAC1 |
| calmodulin | PN | GCG |
| calmodulin | PN | NAPK1 |

TABLE 12

| Protein A | | Protein B |
|---|---|---|
| cAYP | PN | AGT |
| cANP | PN | AKT1 |
| cANP | PN | CXCR4 |
| cAYP | PN | FGF2 |
| cAYP | PN | INHBA |
| cAYP | PN | KITLG |
| cAYP | PN | HPY |
| cANP | PN | SST |
| carbon tetrachloride | PN | CYP2E1 |
| CASP1 | PN | IL18 |
| CASP3 | PN | BIRC5 |
| CASP3 | PN | TGF1 |
| CASP3 | PN | TAC1 |
| CASP3 | PN | TNF |
| CASP7 | PN | BCL2 |
| CASP7 | PN | NAP3K1 |
| CASP8 | PN | TNFATP8 |
| CASP8 | PN | TRAF2 |
| CASP9 | PN | BCL2L1 |
| CASP9 | PN | BIRC4 |
| caspase | PN | IGF1 |
| caspase | PN | IL8 |
| CAT | PN | CYCS |
| catecholamines | PN | CRH |
| catecholamines | PN | LIF |
| catecholamines | PN | NPPA |
| catenin | PN | AR |
| catenin | PN | PTGS2 |
| catenin | PN | TP53 |
| CAV1 | PN | CD36 |
| CAV1 | PN | ESR1 |
| CAV1 | PN | PKA |
| CBL | PN | SH3KBP1 |
| CCAL1 | PN | NAPK1 |
| CCK | PN | CALCA |
| CCK | PN | GCG |
| CCK | PN | MAPK1 |
| CCK | PN | MAPK3 |
| CCK | PN | NGF3 |
| CCK | PN | PLA2G1B |
| CCK | PN | POVC |
| CCK | PN | SST |
| CCL2 | PN | WAPK8 |
| CCL21 | PN | IL10 |
| CCL21 | PN | WAPK1 |
| CCL21 | PN | TNF |
| CCL4 | PN | TGF81 |
| CCL5 | PN | CCL2 |
| CCL5 | PN | IL2 |
| CCL5 | PN | IL8 |
| CCNA2 | PN | CDK6 |
| CCNA2 | PN | CDKN16 |
| CCNB1 | PN | CDKN1A |
| CCND1 | PN | CDK2 |
| CCND1 | PN | CDKN1B |
| CCND1 | PN | CDX1 |
| CCND1 | PN | E2F1 |
| CCND1 | PN | RASSF1 |
| CCND1 | PN | RPS6KB1 |
| CCND1 | PN | TCF4 |
| CCND1 | PN | TGFB1 |
| CCND2 | PN | CDK4 |
| CCND2 | PN | FOKO3A |
| CCND2 | PN | INHBA |
| CCNE1 | PN | CDKN1A |
| CCNE1 | PN | TP53 |
| CCR3 | PN | CCL11 |
| CCR5 | PN | CXCR3 |
| CD14 | PN | IL4 |
| CD14 | PN | TGFB1 |

TABLE 13

| Protein A | | Protein B |
|---|---|---|
| CD24 | PN | CD4 |
| CD28 | PN | CHUK |
| CD28 | PN | IL10 |
| CD28 | PN | TGFB1 |
| CD28 | PN | TNF |
| CD28 | PN | TNFSF6 |
| CD4 | PN | BCL2L1 |
| CD4 | PN | LCK |
| CD44 | PN | LFA-1 (integrin) |
| CD80 | PN | CD28 |
| CD80 | PN | IL10 |
| CD81 | PN | LCK |
| CD86 | PN | IL10 |
| CD8A | PN | CCR5 |
| CD8A | PN | IL10 |
| CD9 | PN | IFNG |
| CDC2 | PN | HIST2H3C |
| CDC2 | PN | RPS6KB1 |
| CDC25A | PN | 14-3-3 |
| CDC25B | PN | CCNA2 |
| CDC25C | PN | ATR |
| CDC25C | PN | CCNA2 |
| CDC42 | PN | AKT1 |
| CDC42 | PN | FegamaRI |
| CDC42 | PN | NAP3K1 |
| CDC42 | PN | NAP3K4 |
| CDC42 | PN | RPS6KB1 |
| CDH2 | PN | YAPK1 |
| CDK2 | PN | CDC25A |
| CDK2 | PN | CDK3 |
| CDK2 | PN | CDKN1B |
| CDK2 | PN | PCNA |
| CDK5 | PN | CDKN1A |
| CDK5 | PN | PSND9 |
| CDK5 | PN | TP53 |
| CDK7 | PN | CCNH |
| CDKN1A | PN | AHPN/CD437 |
| CDKN1A | PN | CCKD2 |
| CDKN1A | PN | Cyp2b20 |
| CDKN1A | PN | FOS |
| CDKN1A | PN | RALGDS |
| CDKN1B | PN | CKS1A |
| CDKN1B | PN | EGF |
| CDKN1A | PN | platelet-derived growth |
| | | factor |
| CDKN1B | PN | PSVD9 |
| CDKN1B | PN | RBL2 |
| CDKN1B | PN | RPS6K |
| CDKN1B | PN | SKP2 |
| CDT1 | PN | GWIH |
| CEBPA | PN | acetyltransferase |
| CEBPA | PN | SP1 |
| CEBPB | PN | NR3C1 |
| CEBPB | PN | TGFB1 |
| CETP | PN | INS |
| CHEK1 | PN | TP53 |
| CHEK2 | PN | TP53 |
| chorionic | PN | TGFA |
| gonadotropin | | |
| CIDEA | PN | DFFA |
| CIDEB | PN | DFFA |
| CLK1 | PN | PTCH |
| CNTF | PN | LIFR |
| CNTF | PN | SOCS3 |
| CNTFR | PN | LIFR |
| COPS5 | PN | JUN |
| cortisol | PN | GH1 |
| cortisol | PN | LEP |
| CREB1 | PN | SOD2 |
| CRH | PN | GH1 |
| CRH | PN | PDNC |
| CRH | PN | SST |

TABLE 14

| Protein A | | Protein B |
|---|---|---|
| CSF1 | PN | COC42 |
| CSF1 | PN | FN1 |
| CSF1 | PN | NYP2 |
| CSF1 | PN | PPARG |
| CSF1 | PN | PTK2 |
| CSF1 | PN | PTPH6 |
| CSF1 | PN | SHC1 |
| CSF2 | PN | IFHA1 |
| CSF2 | PN | IFNG |
| CSF2 | PN | IL10 |
| CSF2 | PN | IL4 |
| CSF2 | PN | IL8 |
| CSF2 | PN | PPARG |
| CSF2 | PN | RARA |
| CSF2 | PN | STAT1 |
| CSF3 | PN | EGR1 |
| CSF3 | PN | EMI1 |
| CSF3 | PN | MAP2X5 |
| CSF3 | PN | Phosphatidylinesitol |
| | | 3-kinase |
| CSF3 | PN | RUNX1 |
| CSF3 | PN | SOCS1 |
| CSF3 | PN | SOCS3 |
| CSF3 | PN | STAT5A |
| CSF3 | PN | TGFB1 |
| CSF3R | PN | STAT1 |
| CSK | PN | PXN |
| CTNNB1 | PN | DKK1 |
| CTNNB1 | PN | TCF4 |
| CTNNB1 | PN | TP53 |
| CTSB | PN | TGFB1 |
| CTTN | PN | SRC |
| CX3CL1 | PN | TLiR1 |
| CX3CR1 | PN | CX3CL1 |
| CXCL1 | PN | PKC |
| CXCL12 | PN | CXCR4 |
| CXCL12 | PN | PTK2B |
| CXCL12 | PN | RAC1 |
| CXCL12 | PN | TNF |
| CXCL13 | PN | IL10 |
| CXCR4 | PN | CCL2 |
| CXCR4 | PN | CCR5 |
| CXCR4 | PN | TL8 |
| cyclase | PN | DRD2 |
| cyclase | PN | NGF3 |
| cyclin | PN | CDH1 |
| cyclin-dependent | PN | BCL2 |
| protein kinase | | |
| cyclin-dependent | PN | CDK2 |
| protein kinase | | |
| cyclin-dependent | PN | CDKN1B |
| protein kinase | | |
| CYCS | PN | BCL2 |
| CYP1A1 | PN | TFNG |
| CYP1A1 | PN | TGFB1 |
| CYP3A4 | PN | CYP2E1 |
| CYRG1 | PN | TP53 |
| cytochrone P450 | PN | TNF |
| dATP/ATP | PN | CASP9 |
| DAXX | PN | MAP3K5 |
| DAXX | PN | MAPK8 |
| DCE | PN | CYP2E1 |
| DCN | PN | RAC1 |
| DCN | PN | RHDA |
| DCN | PN | TNF |
| DIN | PN | CREB1 |
| DIN | PN | TGFA |
| d-lysine | PN | PLAT |
| DNA topoiscaerase | PN | TP53 |
| (ATP hydrolyzing) | | |
| DNA-dependent | PN | ABL1 |
| protein kinase | | |
| DNA-dependent | PN | ATN |
| protein kinase | | |
| DNASE1L3 | PN | PARP1 |
| DNN1 | PN | SRC |
| DNN2 | PN | CTTN |
| DOCK1 | PN | TERF2P |
| dopemine | PN | PTH |
| dopamine D1 receptor | PN | DRD2 |

TABLE 15

| Protein A | | Protein B |
|---|---|---|
| DRD2 | PN | FOS |
| DYL1 | PN | AKT1 |
| DYRK16 | PN | MAPK14 |
| DYRK16 | PN | TCF1 |
| E2 | PN | AKT1 |
| E2 | PN | CASP3 |
| E2F1 | PN | BCL2 |
| E2F1 | PN | BCL2L11 |
| E2F1 | PN | CCHA2 |
| E2F1 | PN | CDKN1A |
| E2F1 | PN | TOPEP1 |
| E2F1 | PN | TP53 |
| E2F4 | PN | RBL2 |
| EAT2 | PN | PGF |
| ECE1 | PN | EDN1 |
| EDN1 | PN | CAV1 |
| EDH1 | PN | DUSP1 |
| EDN1 | PN | GH1 |
| EDK1 | PN | GNA0 |
| EDN1 | PN | LPA |
| EDN1 | PN | MAP2K1 |
| EDN1 | PN | MAPK1 |
| EDN1 | PN | MAPK3 |
| EDN1 | PN | MPPA |
| EDN1 | PN | PTGIS |
| EDN1 | PN | PTHLH |
| EDN1 | PN | SRC |
| EDN1 | PN | src family |
| EDN1 | PN | SST |
| EDNRB | PN | AVP |
| effector caspase | PN | PARP1 |
| EGF | PN | APP |
| EGF | PN | AREG |
| EGF | PN | cANP |
| EGF | PN | CAT |
| EGF | PN | DAB2 |
| EGF | PN | DUSP1 |
| EGF | PN | EPS15 |
| EGF | PN | FGF2 |
| EGF | PN | fibroblast growth factor |
| EGF | PN | FST |
| EGF | PN | GDI |
| EGF | PN | GJA1 |
| EGF | PN | TNHBA |
| EGF | PN | MAP2K2 |
| EGF | PN | MAP3K1 |
| EGF | PN | MAPK14 |
| EGF | PN | PLAU |
| EGF | PN | PLAUR |
| EGF | PN | PRL |
| EGF | PN | PTEN |
| EGF | PN | RAC1 |
| EGF | PN | TERF21P |
| EGF | PN | THBS1 |
| EGFR | PN | catemin |
| EGFR | PN | CAN1 |
| EGFR | PN | DNN1 |
| EGFR | PN | TGF1R |
| EGFR | PN | MAPK14 |
| EGFR | PN | RAS small nonomeric |
| | | GTPase |
| EGFR | PN | SHC3 |
| EGR1 | PN | B-cell receptor |
| EGR1 | PN | JAK2 |
| EGR1 | PN | PPARG |
| EGR1 | PN | TP53 |
| EGTA | PN | SYK |
| ELA2 | PN | CCL2 |
| ELA2 | PN | NNP9 |
| ELA2 | PN | PI3 |
| ELN | PN | NNP2 |
| ELN | PN | NNP9 |
| endothelin | PN | GKRH1 |
| endothelin | PN | NSN3 |
| endothelin | PN | NPPA |
| endothelin | PN | RAF1 |
| endothelin- | PN | EDN1 |
| converting enzyme | | |

TABLE 16

| Protein A | | Protein B |
|---|---|---|
| endotoxin | PN | PTGIS |
| EP300 | PN | CTNNB1 |
| EP300 | PN | IL6 |
| EP300 | PN | IRF1 |
| EP300 | PN | NDY2 |
| EP300 | PN | NYOD1 |
| EP300 | PN | PCKA |
| EP300 | PN | SNAD3 |
| EPHB1 | PN | PXN |
| EPO | PN | CISH |
| EPO | PN | F2 |
| EPO | PN | PTPN6 |
| EPOR | PN | PTPN6 |
| EPSI5 | PN | SNAP25 |
| EPX | PN | JAK2 |
| EPX | PN | JUN |
| EPX | PN | PRKCA |
| EPX | PN | SRC |
| ERBB2 | PN | CSK |
| ERBB2 | PN | EP300 |
| ERBB2 | PN | PPARG |
| ERBB2 | PN | RAF1 |
| ERBB3 | PN | AKT1 |
| ERK activator | PN | MAP2K6 |
| kinase | | |
| ESR1 | PN | CREB1 |
| estradiol | PN | EGF |
| estradiol | PN | LEP |
| estradiol | PN | TGFA |
| estrogen | PN | AGT |
| ETS1 | PN | JUN |
| ETS1 | PN | HYP1 |
| ETS1 | PN | PDGFA |
| ETS1 | PN | TGFB1 |
| F10 | PN | LCP1 |
| F2 | PN | OTR |
| F2 | PN | EGFR |
| F2 | PN | FN1 |
| F2 | PN | IFNG |
| F2 | PN | MAPK1 |
| F2 | PN | PRKOD |
| F2 | PN | PTK2 |
| F2R | PN | map kinase |
| F2R | PN | RPS6K |
| F2R | PN | SRC |
| F3 | PN | FI0 |
| F3 | PN | MAPK3 |
| F7 | PN | IL8 |
| fatty acids | PN | cANP-dependent protein |
| | | kinase. catalyst |
| fatty acids | PN | GCG |
| fatty acids | PN | LEP |
| fatty acids | PN | PPARA |
| FcgamaRI | PN | SYK |
| Fe(ii) | PN | CAT |
| Fe(iii) | PN | HYOX1 |
| FGF1 | PN | EGF |
| FGF1 | PN | IL1 |
| FGF1 | PN | SIOOA13 |
| FGF1 | PN | STAT3 |
| FGF2 | PN | AKT1 |
| FGF2 | PN | ALB |
| FGF2 | PN | CKCL12 |
| FGF2 | PN | F2R |
| FGF2 | PN | FYN |
| FGF2 | PN | GJA1 |
| FGF2 | PN | LCP1 |
| FGF2 | PN | LPA |
| FGF2 | PN | MAPK1 |
| FGF2 | PN | SERPIHE1 |
| FGF2 | PN | SST |

TABLE 17

| Protein A | | Protein B |
|---|---|---|
| FGFR2 | PN | GAP43 |
| fibrin | PN | LCP1 |
| fibrin | PN | PLG |
| fibrinogen | PN | IL1F8 |
| fibrinogen | PN | PTGIS |
| fibrinogen | PN | TNF |
| fibroblast growth | PN | NYP1 |
| factor | | |
| fibroblast growth | PN | NYP3 |
| factor | | |
| fibroblast growth | PN | PRV1 |
| fator | | |
| fibroblast growth | PN | SST |
| factor | | |
| FLT1 | PN | Phosphatidylinositol |
| | | 3-kinase |
| FLT1 | PN | PTK2B |
| FLT4 | PN | FGF2 |
| FN1 | PN | fibrinogen |
| FN1 | PN | GRB2 |
| FN1 | PN | LCP1 |
| FN1 | PN | MAPK3 |
| FN1 | PH | MAPK8 |
| FN1 | PH | NMP14 |
| FN1 | PN | NMP2 |
| FN1 | PN | RAF1 |
| FOSL2 | PN | REL6 |
| FOXA2 | PN | HXF4A |
| FOXA2 | PN | TCF1 |
| FOXO1A | PN | HYOD1 |
| FUT7 | PN | HAID1 |
| GAI7 | PN | IL6 |
| GAB1 | PN | RPS6K |
| GAB2 | PN | AKT1 |
| GABAA receptor | PN | OXT |
| GAL | PN | SST |
| GAP | PN | RAC1 |
| GAPD | PN | BCL2 |
| GAS | PN | GCG |
| GAS | PN | MAPK1 |
| GAS | PN | MAPK3 |
| GAS | PN | NNP9 |
| GAS | PN | HTS |
| GAS | PN | ODC1 |
| GAS | PN | SST |
| GAS6 | PN | AKL |
| GAS6 | PN | STAT3 |
| GATA1 | PN | GATA4 |
| GC6 | PN | INS |
| GC6 | PN | SST |
| GDHF | PN | AKT1 |
| gelatin | PN | NNP2 |
| gelatin | PN | NNP3 |
| GFAP | PN | ITIH4 |
| GH1 | PN | CISH |
| GH1 | PN | GH1 |
| GH1 | PN | SOCS3 |
| GH1 | PN | SST |
| GH1 | PN | TERF2TP |
| GH1 | PN | TNF |
| GH1 | PN | TP53 |
| GHR | PN | IGF1 |
| GHR | PN | SOCS3 |
| GHRH | PN | INS |
| GHRH | PN | SST |
| GHRL | PN | GHRL |
| GHRL | PN | IGF1 |
| GIP | PN | GCG |
| GIP | PN | INS |
| GIP | PN | SST |
| GLI3 | PN | GLI2 |
| glucan 1,4- | PN | INS |
| alpha-glucosidase | | |

TABLE 18

| Protein A | | Protein B |
|---|---|---|
| glucose | PN | BDK |
| glucose | PN | FRAP1 |
| glucose | PN | IGF1 |
| glucose | PN | LEP |
| glucose | PN | MAP2K1 |
| glucose | PN | MAP3K1 |
| glucose | PN | MAPK3 |
| glucose | PN | PRKC0 |
| glucose | PN | SST |
| GLUL | PN | JUN |
| GNA12 | PN | ARHGEF12 |
| GNA12 | PN | BCL2 |
| GNA12 | PN | JUK |
| GNA13 | PN | BCL2 |
| GNA13 | PN | CDC42 |
| GNA13 | PN | JUN |
| GNA14 | PN | SRC |
| GNA0 | PN | MAP2K6 |
| GNA0 | PN | PLCB1 |
| GNLY | PN | GCG |
| GNRH1 | PN | CREB1 |
| GNRH1 | PN | CRH |
| GNRH1 | PN | MAPK3 |
| GNRH1 | PN | POYC |
| GNRH1 | PN | PRL |
| GNRHR | PN | ADCY2 |
| GPX1 | PN | NOS2A |
| GRB2 | PN | HRAS |
| GRIP1 | PN | JUN |
| GRLF1 | PN | HRAS |
| GRLF1 | PN | RAF1 |
| growth factor | PN | ABL1 |
| activity | | |
| GRP | PN | SST |
| GRP | PN | YIP |
| GSK3B | PN | MAPK1 |
| GSN | PN | CFL1 |
| GTF2B | PN | TBPL1 |
| GTP | PN | CDC42 |
| GTP | PN | GHAO |
| GTP | PN | HRAS |
| GTPase | PN | PAK1 |
| guanine nucleotide | PN | CDC42 |
| exchange factor | | |
| GZY8 | PN | BCL2 |
| H1F0 | PN | PLG |
| H2O2 | PN | AKT1 |
| H2O2 | PN | ATV |
| H2O2 | PN | CYCS |
| H2O2 | PN | FGF2 |
| H2O2 | PN | MAP2K1 |
| H2O2 | PN | NGFB |
| H2O2 | PN | PRKCD |
| H2O2 | PN | PTK2 |
| H2O2 | PN | TXK2 |
| HDL | PN | APOB |
| home | PN | HYOX1 |
| home | PN | HSPCA |
| home oxygenase | PN | CAV1 |
| (decyclizing) | | |
| HGF | PN | APP |
| HGF | PN | MAP2K1 |
| HGF | PN | MAPK14 |
| HGF | PN | MAPK3 |
| HGF | PN | MAPK8 |
| HGF | PN | RAC1 |
| HGF | PN | RAF1 |
| HIF1A | PN | NOS2A |
| HIF1A | PN | VEGF |
| histanine receptor | PN | AGT |
| histone deacetylase | PN | ABCB1 |
| histone deacetylase | PN | CCNE1 |
| HLA-A | PN | CD8A |
| HYGB1 | PN | IL1F8 |
| HYOK1 | PN | HSPA4 |
| HOYDI3 | PN | ODC1 |

TABLE 19

| Protein A | | Protein B |
|---|---|---|
| HRAS | PN | AKT1 |
| HRAS | PN | MAPK1 |
| HRAS | PN | MAPK3 |
| HSF1 | PN | HSPCA |
| HSPCA | PN | DCND1 |
| HSPCB | PN | TP53 |
| HTATIP | PN | PTGIS |
| HTATIP | PN | RAF1 |
| HTR2A | PN | PONC |
| HTR2A | PN | PRL |
| hypoxia-inducible | PN | EPX |
| factor 1 | | |
| hypoxia-inducible | PN | KOS2A |
| factor 1 | | |
| ICAN1 | PN | CD4 |
| ICAN1 | PN | MAPK3 |
| ICAN1 | PN | RAF1 |
| ICAN1 | PN | IFAP2A |
| ICOS | PN | IL5 |
| ICOS | PN | TNF |
| IFI16 | PN | TP53 |
| IFNA1 | PN | CISH |
| IFNA1 | PN | IL10 |
| IFNG | PN | COKN1A |
| IFNG | PN | COKN16 |
| IFNG | PN | CISH |
| IFNG | PN | HA |
| IFNG | PN | HGF |
| IFNG | PN | IFNB1 |
| IFNG | PN | INDO |
| IFNG | PN | JAK |
| IFNG | PN | KLRC1 |
| IFNG | PN | LANR1 |
| IFNG | PN | LCP1 |
| IFNG | PN | MAP2K4 |
| IFNG | PN | MAPK1 |
| IFNG | PN | MAPK3 |
| IFNG | PN | NO |
| IFNG | PN | Nuclear factor NF |
| | | kappa B |
| IFNG | PN | PKA |
| IFNG | PN | PKC |
| IFNG | PN | prostaglandin |
| IFNG | PN | RAC1 |
| IFNG | PN | SOCS1 |
| IFNG | PN | SOCS3 |
| IFNG | PN | SST |
| IFNG | PN | testosterone |
| IGBP1 | PN | BCL2 |
| IGF1 | PN | AKT1 |
| TGF1 | PN | BDK |
| IGF1 | PN | EGLAP |
| IGF1 | PN | FN1 |
| IGF1 | PN | GJA1 |
| IGF1 | PN | PRKCE |
| IGF1 | PN | STAT3 |
| IGF1 | PN | STAT5A |
| IGF1R | PN | PKN |
| IGF1R | PN | STAT3 |
| IGF1R | PN | TNF |
| IGF1R | PN | TP53 |
| IGF1R | PN | tumor necrosis factor |
| TGF2 | PN | IGFBP3 |
| IGF2 | PN | IGFB1 |
| IGFBP3 | PN | AGT |
| IGFBP3 | PN | SERPINE1 |
| IGFBP5 | PN | IGF3P6 |
| THH | PN | PTHLH |
| IkappaB kinase | PN | MAPK3 |
| IL1 | PN | AVP |
| IL1 | PN | CHUK |
| IL1 | PN | IFNB1 |
| IL1 | PN | IL1R1 |
| IL1 | PN | MAPK3 |
| IL1 | PN | MAPK9 |
| IL1 | PN | PTGIS |
| IL1 | PN | SST |
| IL1 | PN | TNFRSFIA |
| IL1 | PN | VGAK1 |

TABLE 20

| Protein A | | Protein B |
|---|---|---|
| IL10 | PN | CISH |
| IL10 | PN | INHBA |
| IL10 | PN | SOCS3 |
| IL10 | PN | STAT3 |
| IL10 | PN | THBS1 |
| IL10 | PN | TNFRSF1A |
| IL11 | PN | IFNG |
| IL11 | PN | IL4 |
| IL11 | PN | TNFRSF11B |
| IL15 | PN | IL7 |
| IL17 | PN | CSF3 |
| IL17 | PN | IL4 |
| IL18 | PN | IL4 |
| IL18 | PN | VCAN1 |
| IL1A | PN | IFNB1 |
| IL1A | PN | IL10 |
| IL1A | PN | PONC |
| IL1A | PN | PRL |
| IL1A | PN | TGFB1 |
| IL1A | PN | TNFRSF11B |
| IL1B | PN | BCL2 |
| IL1B | PN | CALCA |
| IL1B | PN | GH1 |
| IL1B | PN | HTATIP |
| IL1B | PN | ICAN1 |
| IL1B | PN | IL13 |
| IL1B | PN | IL4 |
| IL1B | PN | MAP2K1 |
| IL1B | PN | MAP2K6 |
| IL1B | PN | HHP1 |
| IL1B | PN | HHP3 |
| IL1B | PN | NPPA |
| IL1B | PN | OSH |
| IL1B | PN | PONC |
| IL1B | PN | SELE |
| IL1B | PN | SPP1 |
| IL1B | PN | TGFB1 |
| IL1B | PN | TNFRSF11B |
| IL1B | PN | VCAN1 |
| IL1F8 | PN | APP |
| IL1F8 | PN | COKN1A |
| IL1F8 | PN | CSF3 |
| IL1F8 | PN | IL10 |
| IL1F8 | PN | IL1R1 |
| IL1F8 | PN | MAP2K1 |
| IL1F8 | PN | NNP3 |
| IL1F8 | PN | NNP9 |
| IL1F8 | PN | OSH |
| IL1F8 | PN | PPARG |
| IL1F8 | PN | PSYD9 |
| IL1F8 | PN | SNAD3 |
| IL1F8 | PN | SPP1 |
| IL1F8 | PN | TINP1 |
| IL2 | PN | BCL2 |
| IL2 | PN | CISH |
| IL2 | PN | HSPA4 |
| IL2 | PN | TFNA1 |
| IL2 | PN | IL2RA |
| IL2 | PN | JUNB |
| IL2 | PN | LCK |
| IL2 | PN | NNP2 |
| IL2 | PN | PTGIS |
| IL2 | PN | RBL2 |
| IL2 | PN | SOCS3 |
| IL2 | PN | TNFRSF6 |
| IL2 | PN | TP53 |
| IL21 | PN | BCL2 |
| IL2R6 | PN | IL4 |
| IL2R6 | PN | IL7 |
| IL3 | PN | STAT5A |
| IL3 | PN | TGFB1 |

TABLE 21

| Protein A | | Protein B |
|---|---|---|
| IL4 | PN | AKT1 |
| IL4 | PN | CISH |
| IL4 | PN | JAK1 |
| IL4 | PN | JAK2 |
| IL4 | PN | MAPK1 |
| IL4 | PN | NFKBI |
| IL4 | PN | PPARG |
| IL4 | PN | PPPIR136 |
| IL4 | PN | RAF1 |
| IL4 | PN | SOCS3 |
| IL4 | PN | STAT5A |
| IL4 | PN | TNFRSF1A |
| IL4R | PN | interleukin IL13 |
| | | receptor |
| IL5 | PN | CISH |
| IL5 | PN | HRAS |
| IL5 | PN | IL10 |
| IL5 | PN | MAPK1 |
| IL6 | PN | APO8 |
| IL6 | PN | AYP |
| IL6 | PN | BCL2 |
| IL6 | PN | BMP6 |
| IL6 | PN | BYP7 |
| IL6 | PN | CO59 |
| IL6 | PN | CISH |
| IL6 | PN | HDL |
| IL6 | PN | ILEST |
| IL6 | PN | interleukin-6 receptor |
| IL6 | PN | nap kinase |
| IL6 | PN | NVP9 |
| IL6 | PN | NFKBI |
| IL6 | PN | PIAS1 |
| IL6 | PN | PIAS3 |
| IL6 | PN | RAF1 |
| IL6 | PN | RPS6K |
| IL6 | PN | SHOC2 |
| IL6 | PN | STL1 |
| IL6 | PN | SOCS1 |
| IL6 | PN | SOCS2 |
| IL6 | PN | SOCS3 |
| IL6 | PN | SST |
| IL6 | PN | TNF-alpha receptor |
| IL6 | PN | VIP |
| IL7 | PN | IFNG |
| IL7 | PN | IL6 |
| IL8 | PN | BCL2 |
| IL8 | PN | IL1R1 |
| IL8 | PN | NFKBIA |
| IL8 | PN | RHOA |
| IL9 | PN | CISH |
| IL9 | PN | IFNG |
| IL9 | PN | SOCS3 |
| IL9 | PN | STAT5A |
| ILF | PN | SOCS3 |
| ILK | PN | NAFK1 |
| INHBA | PN | INS |
| INHBA | PN | MAPK14 |
| INHBA | PN | MAPK3 |
| inositol | PN | ADCY2 |
| phospholipids | | |
| INS | PN | ABL1 |
| INS | PN | ADRA1A |
| INS | PN | adrenoceptor |
| INS | PN | AGT |
| INS | PN | AGIR2 |
| INS | PN | amylase |
| INS | PN | APOA1 |
| INS | PN | CALCA |
| INS | PN | CRH |
| INS | PN | CRYAB |
| INS | PN | DNH1 |
| INS | PN | DUSP1 |
| INS | PN | FOS |
| INS | PN | GNAD |
| INS | PN | GRB14 |
| INS | PN | GSK38 |
| INS | PN | HSPCA |
| INS | PN | IAPP |
| INS | PN | IGFBP3 |
| INS | PN | IL1 |

TABLE 22

| Protein A | | Protein B |
|---|---|---|
| INS | PN | INPP50 |
| INS | PN | INPPL1 |
| INS | PN | JUN |
| INS | PN | LEP |
| INS | PN | MAPK11 |
| INS | PN | MYC |
| INS | PN | NFKBIA |
| INS | PN | NOS3 |
| INS | PN | OGT |
| INS | PN | opioid receptor |
| INS | PN | PDE3B |
| INS | PN | PIK3R1 |
| INS | PN | PIK3R2 |
| INS | PN | PPARA |
| INS | PN | PRKCD |
| INS | PN | progesterone |
| INS | PN | PTP16 |
| INS | PN | PTPRF |
| INS | PN | RAB4A |
| INS | PN | RAC1 |
| INS | PN | RAPGEF1 |
| INS | PN | RHOO |
| INS | PN | SCN10A |
| INS | PN | SERPINE1 |
| INS | PN | SGK |
| INS | PN | SOCS3 |
| INS | PN | SOCS6 |
| INS | PN | SP1 |
| INS | PN | SST |
| INS | PN | TAT |
| INS | PN | TNFRSF6 |
| INS | PN | TP53INP1 |
| INS | PN | TREP10 |
| INS | PN | TSC1 |
| INS | PN | VANP2 |
| INS | PN | VANP3 |
| insulin receptor | PN | AKT1 |
| insulin receptor | PN | ERS1 |
| insulin receptor | PN | KLK3 |
| insulin receptor | PN | SOCS3 |
| integrin | PN | F3 |
| integrin | PN | MAPK3 |
| interleukin IL12 | PN | IL13 |
| interleukin IL12 | PN | IL4 |
| interleukin IL12 | PN | MAPK1 |
| interleukin IL12 | PN | MAPK8 |
| interleukin IL12 | PN | IL10 |
| receptor | | |
| interleukin IL23 | PN | IFNG |
| interleukin-1 | PN | IFNG |
| receptor ligand | | |
| interleukin-1 | PN | VCAY1 |
| receptor ligand | | |
| LOGAP1 | PN | RAC1 |
| IRF1 | PN | IRF2 |
| IRS1 | PN | AKT1 |
| IRS1 | PN | PPP1R138 |
| IRS1 | PN | RPS6KB1 |
| IRS2 | PN | AKT1 |
| IRS2 | PN | FRAP1 |
| isoproterenol | PN | GCG |
| ITGB1 | PN | SREBF1 |
| ITIH4 | PN | CCL5 |
| ITIHA | PN | MAPK14 |
| JVL | PN | CDKN1A |
| JAK | PN | SOCS3 |
| JAK2 | PN | CISH |
| JAK2 | PN | IFNA1 |
| JAK2 | PN | MAPK8 |
| JAK2 | PN | PRKC0 |
| JAK3 | PN | JAK2 |

TABLE 23

| Protein A | | Protein B |
|---|---|---|
| JAK | PN | DUSP1 |
| JUN | PN | CEBPG |
| JUN | PN | OKK1 |
| JUN | PN | EBP |
| JUN | PN | GSK3B |
| JUN | PN | NR4A1 |
| JUN | PN | Phosphatidylinositol |
| | | 3-kinase |
| JUN | PN | RARA |
| JUN | PN | SO02 |
| JUN | PN | SP11 |
| JUK8 | PN | NPPA |
| KDR | PN | AKT1 |
| KDR | PN | CAV1 |
| KDR | PN | HSPCA |
| KDR | PN | TGFB1 |
| kininogenase | PN | BDK |
| KIT | PN | BCL2L1 |
| KIT | PN | MYC |
| KITLG | PN | AKT1 |
| KITLG | PN | BCL2L1 |
| KITLG | PN | CISH |
| KITLG | PN | IL8 |
| KITLG | PN | LYN |
| KITLG | PN | SRC |
| KLK3 | PN | A2Y |
| KLRA1 | PN | NIC8 |
| KRAS2 | PN | AKT1 |
| LAN | PN | IL10 |
| laminin | PN | NYP2 |
| laminin | PN | PLAU |
| laminin | PN | PONC |
| LBP | PN | CD14 |
| LCK | PN | MAPK1 |
| LCP1 | PN | A2Y |
| LCP1 | PN | MP3 |
| LCP2 | PN | PLCG1 |
| LDL | PN | ALB |
| LDL | PN | YAPK14 |
| LDL | PN | RHOA |
| LDL | PN | SELE |
| LEP | PN | CISH |
| LEP | PN | POYC |
| LEP | PN | SOCS3 |
| LEP | PN | STAT3 |
| LEP | PN | TGFB1 |
| LGALS1 | PN | PTPRC |
| LIF | PN | CISH |
| LIF | PN | MAPK8 |
| linoleic acid | PN | EGFR |
| lipid | PN | PPARA |
| Lipids | PN | AKT1 |
| Lipopolysaccharide | PN | IL10 |
| LOC365454 | PN | RAC1 |
| LPA | PN | CAT |
| LPA | PN | DTR |
| LPA | PN | EGFR |
| LPA | PN | GAB1 |
| LPA | PN | PLG |
| LPA | PN | PTPN11 |
| LPL | PN | LDLR |
| LPL | PN | PRL |
| LPS | PN | HTATIP |
| LPS | PN | IFNA1 |
| LPS | PN | IFNG |
| LPS | PN | IL10 |
| LPS | PN | IL6 |
| LPS | PN | MAPK9 |
| LPS | PN | SPP1 |
| LPS | PN | TGFB1 |
| LRBA | PN | TP53 |
| LRP1 | PN | VEGF |
| LTB | PN | PPARA |
| LTB4 | PN | IL4 |

TABLE 24

| Protein A | | Protein B |
|---|---|---|
| Luteinizing hormone | PN | IL1B |
| LYN | PN | MAP2K7 |
| LYN | PN | protein phosphatase 1 |
| MAG-1 (integrin) | PN | CAT |
| MADD | PN | TNF |
| MAH | PN | CYP2E1 |
| map kinase | PN | CALCA |
| map kinase | PN | CREB1 |
| map kinase | PN | DUSP1 |
| map kinase | PN | HSPA4 |
| map kinase | PN | SELL |
| map kinase | PN | SOCS3 |
| map kinase | PN | TH |
| MAP kinase kinase | PN | TERF2IP |
| kinase | | |
| MAP2K1 | PN | CDG2PN |
| MAP2K1 | PN | MAPK14 |
| MAP2K1 | PN | MAPK8 |
| MAP2K1 | PN | SNAD2 |
| MAP2K2 | PN | MAPK14 |
| MAP2K2 | PN | MAPK8 |
| MAP2K3 | PN | MAPK1 |
| MAP3K1 | PN | AXT1 |
| MAP3K1 | PN | DUSP1 |
| MAP3K1 | PN | MAP2K4 |
| MAP3K1 | PN | MAPK9 |
| MAP3K1 | PN | THFRSF6 |
| MAP3K10 | PN | MAP2K4 |
| MAP3K3 | PN | CHUK |
| MAP3K5 | PN | CDC25A |
| MAP3K5 | PN | TP53 |
| MAPK1 | PN | ADRBK1 |
| MAPK1 | PN | AR |
| MAPK1 | PN | ATF2 |
| MAPK1 | PN | CASP3 |
| MAPK1 | PN | CDK4 |
| MAPK1 | PN | CISH |
| MAPK1 | PN | DEFB4 |
| MAPK1 | PN | DUSP1 |
| MAPK1 | PN | DUSP4 |
| MAPK1 | PN | HEF1A |
| MAPK1 | PN | MLC1 |
| MAPK1 | PN | MYP2 |
| MAPK1 | PN | nadph oxidase |
| MAPK1 | PN | PLAUR |
| MAPK1 | PN | protein-glutamine gamre- |
| | | glutanyltransferase |
| MAPK1 | PN | RPS6 |
| MAPK1 | PN | SOCS1 |
| MAPK14 | PN | CDKH1A |
| MAPK14 | PN | HSPA4 |
| MAPK14 | PN | nadph oxidase |
| MAPK14 | PN | NOS3 |
| MAPK14 | PN | NPPA |
| MAPK14 | PN | PPARA |
| MAPK14 | PN | PPARG |
| MAPK14 | PN | SWD3 |
| MAPK3 | PN | CDKN1B |
| MAPK3 | PN | dual-specificity |
| | | protein phosphatase |
| MAPK3 | PN | FOSB |
| MAPK3 | PN | GAB1 |
| MAPK3 | PN | MAPKAP1 |
| MAPK3 | PN | MCF1 |
| MAPK3 | PN | PRKCE |
| MAPK3 | PN | PTGS2 |
| MAPK3 | PN | SAP kinase |
| MAPK7 | PN | MAP2K2 |
| MAPK7 | PN | MAPK3 |

TABLE 25

| Protein A | | Protein B |
|---|---|---|
| MAPK8 | PN | CAT |
| MAPK8 | PN | CDKN1A |
| MAPK8 | PN | DUSP1 |
| MAPK8 | PN | HSP81 |
| MAPK8 | PN | IkappaB kinase |
| MAPK8 | PN | JKK |
| MAPK8 | PN | MPPB |
| MAPK8 | PN | PARP1 |
| MAPK8 | PN | PKC |
| MAPK8 | PN | PPN1L |
| MAPK8 | PN | protein phosphatase 2A |
| MAPK8 | PN | SYAD2 |
| MAPK8 | PN | SYCA |
| MAPK8 | PN | SOD2 |
| MAPK8 | PN | ATF2 |
| MAPK9 | PN | SCL2L1 |
| MAPK9 | PN | MAPK8 |
| MAPK9 | PN | SNAD4 |
| MAPKAP1 | PN | [symbol] OX1 |
| matrix | PN | [symbol] P2 |
| metalloproteinase | | |
| MAX | PN | NXI1 |
| MCF2 | PN | CDC42 |
| MCF2 | PN | MAPK1 |
| MCF2L | PN | CDC42 |
| MCN7 | PN | NC [symbol] 2 |
| MCP | PN | TGFB1 |
| MDA | PN | CAT |
| MDK2 | PN | E2F1 |
| MDK2 | PN | HIF1A |
| MDK2 | PN | MDH4 |
| MEN1 | PN | RUNX2 |
| MET | PN | PRL |
| MIF | PN | src family |
| MITF | PN | MAPK14 |
| MKNK1 | PN | EIF4EBP1 |
| MLN | PN | INS |
| MNP13 | PN | TINP1 |
| MYP14 | PN | VEGF |
| MYP2 | PN | map kinase |
| MYP2 | PN | PTK2 |
| MYP2 | PN | TINP1 |
| MYP3 | PN | FGF7 |
| MYP3 | PN | JUN |
| MYP3 | PN | TIYP1 |
| MOS | PN | RPS6K |
| MSIB | PN | EGFR |
| MSTI | PN | DFFA |
| MTBP | PN | IP53 |
| MUC4 | PN | SNAD2 |
| MYC | PN | CD [symbol] 2A |
| MYC | PN | GAPD |
| NYC | PN | GATA1 |
| NYC | PN | histone |
| NYC | PN | KRT18 |
| NYC | PN | NFKB1A |
| NYC | PN | NSEP1 |
| NYC | PN | phosphopyruvate |
| | | hydratase |
| NYC | PN | TFAP2A |
| NYCN | PN | NGFB |
| NYOD1 | PN | TGFB1 |
| NCF1 | PN | MAPK1 |
| NCSTN | PN | APP |
| nerve growth factor | PN | TP53 |
| receptor ligand | | |
| NF-kappeB-inducing | PN | MAPK8 |
| kinase | | |
| NFKBIA | PN | RELA |
| NFKBIA | PN | SRC |
| NSFB | PN | AKT1 |
| NGFB | PN | EGR1 |
| NGFB | PN | IFNG |
| NGFB | PN | LIF |
| NGFB | PN | MAPK8 |
| NGFB | PN | PLAUR |
| NGFB | PN | PPP1R13B |
| NGFB | PN | RAC1 |
| NGFB | PN | TP53 |

TABLE 26

| Protein A | | Protein B |
|---|---|---|
| NID | PN | NKP3 |
| nitric oxide | PN | SRC |
| synthase | | |
| N [symbol] DA receptor | PN | PARP1 |
| NNK | PN | CγP2E1 |
| NNK | PN | PTGS1 |
| NO | PN | BCL2 |
| NO | PN | BCL2L1 |
| NO | PN | GCG |
| NO | PN | GNRH1 |
| NO | PN | HRAS |
| NO | PN | IL10 |
| NO | PN | SPP1 |
| NO | PN | TP53 |
| noradrenaline | PN | AVP |
| norapinephrine | PN | APP |
| NOS1 | PN | PTGS2 |
| NOS2A | PN | HNGB1 |
| NOS2A | PN | IL1R1 |
| NOS2A | PN | JUN |
| NOS2A | PN | NFNB1A |
| NOS2A | PN | TP53 |
| NOS3 | PN | SRC |
| NOTCH3 | PN | VAPK1 |
| NPY | PN | CRH |
| NROB1 | PN | NR5A1 |
| NR3C1 | PN | BCL2L1 |
| NR3C1 | PN | HSPCA |
| NR3C1 | PN | KY3 |
| NR3C1 | PN | NFKBIA |
| NR5A2 | PN | NROB2 |
| NRG1 | PN | NAPK14 |
| NTS | PN | DRD2 |
| NTS | PN | SST |
| NUCB2 | PN | PPARA |
| Nuclear factor NF | PN | AHR |
| kappa B | | |
| Nuclear factor NF | PN | CISH |
| kappa B | | |
| Nuclear factor NF | PN | EBP |
| kappa B | | |
| Nuclear factor NF | PN | EGR1 |
| kappa B | | |
| Nuclear factor NF | PN | F3 |
| kappa B | | |
| Nuclear factor NF | PN | FOS |
| kappa B | | |
| Nuclear factor NF | PN | HYOX1 |
| kappa B | | |
| Nuclear factor NF | PN | IRF1 |
| kappa B | | |
| Nuclear factor NF | PN | NYC |
| kappa B | | |
| Nuclear factor NF | PN | NOS2A |
| kappa B | | |
| Nuclear factor NF | PN | REL |
| kappa B | | |
| okadaic acid | PN | IL1R1 |
| opioid receptor | PN | NAPK1 |
| OSN | PN | CDKN1A |
| OSN | PN | STAT3 |
| OXT | PN | POYC |
| oxygen | PN | BOL2L1 |
| oxygen | PN | SELP |
| Oxysterols | PN | CAT |
| PAH | PN | CYP1A1 |
| PAK2 | PN | ABL1 |
| PARP1 | PN | CASP7 |
| PARP1 | PN | CDKN1A |
| PARP1 | PN | NAPK1 |

TABLE 27

| Protein A | | Protein B |
|---|---|---|
| pathway-specific | PN | JUN |
| SYAD protein | | |
| pathway-specific | PN | NAPK8 |
| SYAD protein | | |
| pathway-specific | PN | SYAD2 |
| SYAD protein | | |
| PAKR | PN | CASP8 |
| PAKR | PN | YAPK1 |
| PAKR | PN | RAF1 |
| PAKR | PN | TNFRSF6 |
| PAX6 | PN | INS |
| PC4 | PN | CDKN1B |
| PCNA | PN | CDKN1A |
| PDCD8 | PN | DIABL0 |
| PDE40 | PN | NAPK1 |
| PDGFA | PN | NAP2K1 |
| PDGFRB | PN | PLCG1 |
| pentagastrin | PN | CCK |
| peptide receptor, | PN | CXCL12 |
| G-protein coupled | | |
| peptide receptor, | PN | CKCR3 |
| G-protein coupled | | |
| peptide receptor, | PN | RAC1 |
| G-protein coupled | | |
| PF4 | PN | IL8 |
| PGE1 | PN | IL10 |
| PGE1 | PN | IL4 |
| PGE1 | PN | PRL |
| PGE2 | PN | BNP2 |
| PGE2 | PN | GH1 |
| PGE2 | PN | IL1R1 |
| PGE2 | PN | NPPA |
| PGF | PN | BHOA |
| PGF2 alpha | PN | PRL |
| phenylephrine | PN | NPPA |
| phosphatidylcholine | PN | CCL21 |
| phosphatidylcholine | PN | F2 |
| Phosphatidylinositol | PN | F3 |
| 3-kinas | | |
| Phosphatidylinositol | PN | CTSB |
| 3-kinase | | |
| Phosphatidylinositol | PN | EIF4E |
| 3-kinase | | |
| Phosphatidylinositol | PN | EPHA3 |
| 3-kinase | | |
| Phosphatidylinositol | PN | HIF1A |
| 3-kinase | | |
| Phosphatidylinositol | PN | YDK2 |
| 3-kinase | | |
| Phosphatidylinositol | PN | NEF2A |
| 3-kinase | | |
| Phosphatidylinositol | PN | PIPN11 |
| 3-kinase | | |
| phosphoinositide | PN | GRP58 |
| PI | PN | NSHB |
| PIK3CA | PN | AKT1 |
| PIK3CA | PN | PPP1R13B |
| PIK3CG | PN | RAC1 |
| PIK3R1 | PN | Phosphatidylinositol |
| | | 3-kinase |
| PIN1 | PN | STAT5A |
| PKA | PN | AKT1 |
| PKA | PN | HRAS |
| PKA | PN | HSFB1 |
| PKA | PN | IGF1 |
| PKA | PN | NFKB1A |
| PKC | PN | DUSP1 |
| PKC | PN | EIF4EBP1 |
| PKC | PN | NET |
| PKC | PN | NPPA |
| PKC | PN | RB1 |
| PKC | PN | SELP |
| PKC | PN | SRC |
| PLA2G2D | PN | NAPK1 |

TABLE 28

| Protein A | | Protein B |
|---|---|---|
| PLAT | PN | LPA |
| PLAT | PN | PLG |
| PLAT | PN | SERPINE1 |
| platelet-derived | PN | ADCY2 |
| growth factor | | |
| platelet-derived | PN | ENP2 |
| growth factor | | |
| platelet-derived | PN | CAT |
| growth factor | | |
| platelet-derived | PN | DUSP1 |
| growth factor | | |
| platelet-derived | PN | GJA1 |
| growth factor | | |
| platelet-derived | PN | KLK3 |
| growth factor | | |
| platelet-derived | PN | NAP3K11 |
| growth factor | | |
| platelet-derived | PN | NAPK3 |
| growth factor | | |
| platelet-derived | PN | NYC |
| growth factor | | |
| platelet-derived | PN | NGFB |
| growth factor | | |
| platelet-derived | PN | PPARG |
| growth factor | | |
| platelet-derived | PN | PTGIS |
| growth factor | | |
| platelet-derived | PN | PTPN11 |
| growth factor | | |
| platelet-derived | PN | RAC1 |
| growth factor | | |
| platelet-derived | PN | RAF1 |
| growth factor | | |
| platelet-derived | PN | TXN |
| growth factor | | |
| platelet-derived | PN | EDN1 |
| growth factor | | |
| platelet-derived | PN | NPPA |
| growth factor | | |
| platelet-derived | PN | VEGF |
| growth factor | | |
| PLAU | PN | NYP3 |
| PLAU | PN | PLAT |
| PLAU | PN | SRC |
| PLAUR | PN | SERPINE1 |
| PLCG1 | PN | GSN |
| PLCG1 | PN | PRKCN |
| PLCG1 | PN | profilin |
| PLG | PN | ELA2 |
| PNA | PN | ADCY2 |
| PNA | PN | ADN |
| PNA | PN | CCND1 |
| PNA | PN | DTR |
| PNA | PN | OUSP1 |
| PNA | PN | ESR1 |
| PNA | PN | NAP3K1 |
| PNA | PN | NAPK1 |
| PNA | PN | NAPK3 |
| PNA | PN | YYC |
| PNA | PN | NPPA |
| PNA | PN | PAK1 |
| PNA | PN | RAF1 |
| PNA | PN | SST |
| PHCH | PN | GNA0 |
| PNL | PN | TP53 |
| porin | PN | BCL2L1 |
| PPARG | PN | EGF |
| PPARG | PN | IRF1 |
| PPARG | PN | NAPK1 |
| PPARG | PN | NAPK3 |
| PPARG | PN | NAPK3 |
| PPARG | PN | TGFB1 |
| PRKCA | PN | TP53 |
| PRKCE | PN | calcium |
| PRKCE | PN | nap kinase |
| PRKCE | PN | RPSGK |
| PRKCZ | PN | RPSGKB1 |
| PRKDC | PN | TP53 |

TABLE 29

| Protein A | | Protein B |
|---|---|---|
| PRL | PN | AKT1 |
| PRL | PN | CESH |
| PRL | PN | IFKG |
| PRL | PN | IGF2 |
| PRL | PN | JUN |
| PRL | PN | LIF |
| PRL | PN | NOS2A |
| PRL | PN | TNF |
| progesterone | PN | DUSP1 |
| progesterone | PN | GNRH1 |
| progesterone | PN | HYGCR |
| progesterone | PN | LEP |
| prostaglandin | PN | EGFR |
| prostaglandin | PN | POEC |
| prostaglandin | PN | PRL |
| prostaglandin- | PN | VIP |
| endoperox de synthase | | |
| protein C | PN | F2R |
| (activated) | | |
| protein phosphatase | PN | F2 |
| protein phosphatase | PN | IL6 |
| protein | PN | RAF1 |
| phosphatase 1 | | |
| protein | PN | RB1 |
| phosphatase 1 | | |
| protein | PN | SP1 |
| phosphatase 2A | | |
| protein tyrosine | PN | BCL2L1 |
| kinase | | |
| protein tyrosine | PN | BDK |
| kinase | | |
| protein tyrosine | PN | CBL |
| kinase | | |
| protein tyrosine | PN | [symbol] RS1 |
| kinase | | |
| protein tyrosine | PN | NPPA |
| kinase | | |
| protein tyrosine | PN | PTK28 |
| kinase | | |
| protein tyrosine | PN | SOCS3 |
| kinase | | |
| protein tyrosine | PN | NYC |
| kinase | | |
| protein tyrosine | PN | PXN |
| phosphatase | | |
| PSD | PN | RAC1 |
| PSEN1 | PN | APP |
| PSYD9 | PN | Cyp2b20 |
| PTEN | PN | FOX01A |
| PTEN | PN | oxygen |
| PTGDS | PN | PPARG |
| PTGER1 | PN | IL1B |
| PTGIS | PN | IL10 |
| PTGS1 | PN | PTGS2 |
| PTGS2 | PN | IL4 |
| PTGS2 | PN | NFKBIA |
| PTGS2 | PN | PPARA |
| PTGS2 | PN | PTGIS |
| PTH | PN | IL18 |
| PTHLH | PN | EGFR |
| PTHLH | PN | PTH |
| PTHR1 | PN | IGF1 |
| PTK2 | PN | BCAR1 |
| PTK2 | PN | CASP3 |
| PTK2 | PN | CASP3 |
| PTK2 | PN | NAPK3 |
| PTK2 | PN | PTEN |
| PTK2 | PN | PTPN11 |
| PTK2 | PN | PKH |
| PTK2B | PN | RHOA |
| PTP4A3 | PN | JAK2 |
| PTPRA | PN | SRC |
| PTPRC | PN | STAT6 |
| PTTGI | PN | TP53 |

TABLE 30

| Protein A | | Protein B |
|---|---|---|
| quinone | PN | TP53 |
| RA | PN | CYP1A1 |
| RAC1 | PN | COH1 |
| RAC1 | PN | map kinase |
| RAC1 | PN | VAP3K1 |
| RAC1 | PN | NAPK14 |
| RAC1 | PN | NNP2 |
| RAC1 | PN | RHOA |
| RAC1 | PN | SAP kinase |
| RAF1 | PN | BRAF |
| RAF1 | PN | RPSGKB1 |
| RALGDS | PN | STAT3 |
| RAPGEF1 | PN | RAF1 |
| RARA | PN | IGFBP3 |
| RAS small monomeric | PN | CCHD1 |
| GTPase | | |
| RAS small monomeric | PN | CDKN2A |
| GTPase | | |
| RAS small monomeric | PN | E2F1 |
| GTPase | | |
| RAS small monomeric | PN | VAP2K6 |
| GTPase | | |
| RAS small monomeric | PN | VAPK14 |
| GTPase | | |
| RAS small monomeric | PN | PAK1 |
| GTPase | | |
| RAS small monomeric | PN | PPPIR13B |
| GTPase | | |
| RAS small monomeric | PN | TRAF3 |
| GTPase | | |
| RASA1 | PN | GRLF1 |
| RASD1 | PN | NAPK3 |
| RB1 | PN | CDKN1A |
| RB1 | PN | COKN1B |
| RB1 | PN | SP1 |
| RB1 | PN | TGFB2 |
| RBL1 | PN | CCND1 |
| RBL1 | PN | CDKN1A |
| RBL2 | PN | CCNE1 |
| receptor signaling | PN | AKT1 |
| protein | | |
| receptor tyrosine | PN | PTPN6 |
| kinase | | |
| REL | PN | BCL2L1 |
| REL | PN | RELB |
| RELA | PN | EP300 |
| RELB | PN | NFKBIA |
| REH | PN | ARG2 |
| REN | PN | NPY |
| RET | PN | AKT1 |
| RETN | PN | AKT1 |
| RETN | PN | EDN1 |
| RGS2 | PN | IL2 |
| RHO | PN | JUN |
| RHO | PN | GRK1 |
| Rho small monomeric | PN | CDCA2 |
| GTPase | | |
| Rho small monomeric | PN | NAPK8 |
| GTPase | | |
| Rho small monomeric | PN | PTK2 |
| GTPase | | |
| Rho small monomeric | PN | RAC1 |
| GTPase | | |
| Rho small monomeric | PN | RHOA |
| GTPase | | |
| RHOA | PN | geranylgeranyl |
| | | pyrophosphate |
| RHOA | PN | map kinase |
| RHOA | PN | NAPK1 |
| RHOA | PN | NKL1 |
| RHOA | PN | NYOD1 |
| RHOA | PN | oxygen |
| RHOA | PN | PAKG |
| RHOA | PN | PCLD |
| RHOA | PN | RAF1 |
| RHOA | PN | TNC |
| RIPK1 | PN | MAP3K1 |
| RIPK2 | PN | MAPK1 |
| RIPK2 | PN | MAPK3 |

TABLE 31

| Protein A | | Protein B |
|---|---|---|
| RPS6K | PN | CALCA |
| RPS6K | PN | CREB1 |
| RPS6K | PN | DUSP1 |
| RPS6K | PN | EIF4E |
| RPS6K | PN | HSPA4 |
| RPS6K | PN | SELL |
| RPS6K | PN | TERF21P |
| RPS6K | PN | TH |
| RPS6K81 | PN | MAPKS |
| RRAS | PN | HRAS |
| RRAS | PN | PTK2 |
| RRAS | PN | RAF1 |
| RUNX2 | PN | TNFSF11 |
| SI00B | PN | IFN6 |
| SIP | PN | RAC1 |
| SAP kinase | PN | BCL2 |
| SAP kinase | PN | DUSP1 |
| SAP kinase | PN | IL6 |
| SAP kinase | PN | IL8 |
| SCARB1 | PN | CAV1 |
| SCLY | PN | CSF2 |
| SCT | PN | INS |
| SELE | PN | STAT6 |
| SELL | PN | MAPK8 |
| SELPLG | PN | PKC |
| SERPINE1 | PN | MAPK3 |
| SERPINE2 | PN | F2R |
| SHC1 | PN | MAPK3 |
| SHC1 | PN | PTEN |
| SHH | PN | BHP2 |
| SLC12A9 | PN | RB1 |
| SLC2A4 | PN | SLC2A1 |
| SLJT2 | PN | CXCL12 |
| SMAD1 | PN | SMAD4 |
| SMAD3 | PN | ESR1 |
| SMAD3 | PN | JUN |
| SMAD3 | PN | MAP3K1 |
| SMAD3 | PN | NR3C1 |
| SMAD3 | PN | SMAD7 |
| SMAD3 | PN | MDR |
| SKAD4 | PN | MAPK3 |
| SMAD5 | PN | SMAD9 |
| small numeric | PN | RAF1 |
| GTPase | | |
| SMAP | PN | MAPK1 |
| SOD2 | PN | PGE2 |
| SP1 | PN | CC2D1 |
| SP1 | PN | ILIF8 |
| SP1 | PN | IRF1 |
| SP1 | PN | MUC2 |
| SP1 | PN | THFRSF6 |
| SP1 | PN | TP53 |
| sphingosine-1- | PN | TGF81 |
| phosohate phosphatase | | |
| SPN | PN | TNF |
| SPP1 | PN | MAPK1 |
| SRC | PN | ADRBK1 |
| SRC | PN | ATF2 |
| SRC | PN | CAV1 |
| SRC | PN | CCL21 |
| SRC | PN | GRB2 |
| SRC | PN | HRAS |
| SRC | PN | MAP2K2 |
| SRC | PN | NYP2 |
| SRC | PN | NYP9 |
| SRC | PN | PLAUR |
| src family | PN | GAV1 |
| src family | PN | CBL |
| src family | PN | HRAS |
| src family | PN | LYN |
| src family | PN | MAPK9 |
| src family | PN | PLAUR |
| SREBF1 | PN | HYGCR |
| SREBF1 | PN | LDLR |
| SREBF1 | PN | LEP |
| SRF | PN | IER2 |
| SST | PN | CDC25C |
| SST | PN | IL2 |
| SSTR2 | PN | PTPN11 |
| SSTR2 | PN | PTPN6 |

TABLE 32

| Protein A | | Protein B |
|---|---|---|
| STAT3 | PN | CISH |
| STAT3 | PN | JUN |
| STAT3 | PN | SOCS3 |
| STAT5A | PN | ESR1 |
| STAT5B | PN | SOCS3 |
| STAT6 | PN | IFN6 |
| steroid receptor | PN | PGR |
| STNN1 | PN | TP53 |
| STX4A | PN | F2 |
| superoxide cisutase | PN | INS |
| SYK | PN | MPK3 |
| T-cell transcription | PN | GSK3B |
| factor NFATC | | |
| T-cell transcription | PN | MAPK8 |
| factor NFATC | | |
| TBP | PN | TP53 |
| TCE | PN | CYP1A1 |
| TCE | PN | CYP2E1 |
| TCE | PN | Cyp3a11 |
| T-cell receptor | PN | CBLB |
| T3 complex | | |
| T-cell receptor | PN | JUN |
| T3 complex | | |
| T-cell receptor | PN | MAP2K1 |
| T3 complex | | |
| T-cell receptor | PN | MAP3K1 |
| T3 complex | | |
| T-cell receptor | PN | RAC1 |
| T3 complex | | |
| TCF1 | PN | ALB |
| TDE1 | PN | CASP9 |
| telomerase | PN | MAPK1 |
| testosterone | PN | CYP19A1 |
| testosterone | PN | TAC1 |
| testosterone | PN | TGFB1 |
| TFDP1 | PN | E2F1 |
| TFF3 | PN | MAPK1 |
| TFRC | PN | TF |
| TG | PN | IL1B |
| TGFB1 | PN | activin |
| TGFB1 | PN | BGN |
| TGFB1 | PN | COL21 |
| TGFB1 | PN | CTF1 |
| TGFB1 | PN | DPT |
| TGFB1 | PN | FST |
| TGFB1 | PN | HYAL2 |
| TGFB1 | PN | IGFBP2 |
| TGFB1 | PN | IGFBP5 |
| TGFB1 | PN | IL7 |
| TGFB1 | PN | inhibitory SMAD protein |
| TGFB1 | PN | KLRC1 |
| TGFB1 | PN | LAMA1 |
| TGFB1 | PN | LOK |
| TGFB1 | PN | LPA sodium sell |
| TGFB1 | PN | MAP2K4 |
| TGFB1 | PN | NF1 |
| TGFB1 | PN | NPPB |
| TGFB1 | PN | PGE1 |
| TGFB1 | PN | PLAS4 |
| TGFB1 | PN | PKG |
| TGFB1 | PN | PLA2G1B |
| TGFB1 | PN | PLAUR |
| TGFB1 | PN | PPARA |
| TGFB1 | PN | procollagen |
| | | N-endopeptidase |
| TGFB1 | PN | RA |
| TGFB1 | PN | RAC1 |
| TGFB1 | PN | REL |
| TGFB1 | PN | SERPINE1 |
| TGFB1 | PN | SHADG |
| TGFB1 | PN | SMAD7 |
| TGFB1 | PN | THBS2 |
| TGFBR1 | PN | SMAD3 |
| THBD | PN | PROC |
| THBS1 | PN | MNP2 |
| THP0 | PN | STAT1 |
| THP0 | PN | STAT3 |
| THP0 | PN | TNF |

TABLE 33

| Protein A | | Protein B |
|---|---|---|
| thrombin | PN | DTR |
| thrombin | PN | EGFR |
| thrombin | PN | FN1 |
| thrombin | PN | PRKOD |
| thrombin | PN | PRKCE |
| thrombin | PN | PTGTS |
| thrombin | PN | PTK2 |
| thrombin | PN | SERPINE1 |
| thyroid stimulating | PN | STAT3 |
| hormone | | |
| TIKP1 | PN | MAPK14 |
| TLR4 | PN | IL10 |
| TLR4 | PN | IL6 |
| TLR4 | PN | TNF |
| TNA | PN | PLG |
| TNC | PN | HYP13 |
| TNF | PN | ADH |
| TNF | PN | ANXA1 |
| TNF | PN | ATF3 |
| TNF | PN | BCL2A1 |
| TNF | PN | BCL2L1 |
| TNF | PN | BCL3 |
| TNF | PN | CD4 |
| TNF | PN | CD80 |
| TNF | PN | CD8A |
| TNF | PN | CHUX |
| TNF | PN | CTNNB1 |
| TNF | PN | CXCL9 |
| TNF | PN | DUSP1 |
| TNF | PN | E2 |
| TNF | PN | E2F1 |
| TNF | PN | exo-alpha-sialidase |
| TNF | PN | ferritin |
| TNF | PN | FGF2 |
| TNF | PN | HDL |
| TNF | PN | HIF1A |
| TNF | PN | HRAS |
| TNF | PN | IFNB1 |
| TNF | PN | Ikappa B |
| TNF | PN | ILIORA |
| TNF | PN | IL11 |
| TNF | PN | IL9 |
| TNF | PN | IRF1 |
| TNF | PN | ITGA4 |
| TNF | PN | LEF |
| TNF | PN | LFA-1 (integrin) |
| TNF | PN | MAP2K1 |
| TNF | PN | MAP2K2 |
| TNF | PN | MAP2K4 |
| TNF | PN | MAP3K1 |
| TNF | PN | MAP3K5 |
| TNF | PN | MAP3K3 |
| TNF | PN | HSK1 |
| TNF | PN | ND |
| TNF | PN | NR3C1 |
| TNF | PN | PDGFA |
| TNF | PN | P13 |
| TNF | PN | PPARA |
| TNF | PN | PRKCI |
| TNF | PN | protein tyrosine kinase |
| | | inhibitor |
| TNF | PN | PTGS2 |
| TNF | PN | RAC1 |
| TNF | PN | RHOA |
| TNF | PN | SAP kinase |
| TNF | PN | SERPING1 |
| TNF | PN | SHAD1 |
| TNF | PN | SOCS1 |
| TNF | PN | SOD1 |
| TNF | PN | SOD2 |
| TNF | PN | SPHK1 |
| TNF | PN | STAT1 |
| TNF | PN | STAT6 |
| TNF | PN | superoxide dismutase |
| TNF | PN | TLR2 |
| TNF | PN | TNFRSF1A |
| TNF | PN | TRAD0 |
| TNF | PN | TRAF1 |
| TNF | PN | TRAF4 |
| TNF | PN | TXN |
| TNF | PN | VIP |
| TNF | PN | ZFP36 |

TABLE 34

| Protein A | | Protein B |
|---|---|---|
| TNFATPB | PN | NFKBIA |
| TNF-alpha receptor | PN | NFKBIA |
| TNFRSF108 | PN | CASP10 |
| TNFRSF1A | PN | CASP3 |
| TNFRSF1A | PN | VAPK14 |
| TNFRSF5 | PN | CD86 |
| TNFRSF5 | PN | STAT6 |
| TNFRSF6 | PN | OOKNIA |
| TNFRSF6 | PN | IL10 |
| TNFRSF6 | PN | IL3 |
| TNFRSF6 | PN | NAPK14 |
| TNFRSF6 | PN | BAPK9 |
| TNFRSF6 | PN | NFKB1 |
| TNFRSF6 | PN | PPARA |
| TNFRSF6 | PN | RAC1 |
| TNFRSF10 | PN | BCL2 |
| TNFRSF10 | PN | BCL2L1 |
| TNFRSF10 | PN | CYCS |
| TNFRSF10 | PN | EGF |
| TNFRSF10 | PN | NAPK1 |
| TNFRSF10 | PN | NFKBIA |
| TNFRSF10 | PN | PARP1 |
| TNFRSF10 | PN | TKF |
| TNFRSF11 | PN | HFKBIA |
| TNFRSF4 | PN | IL4 |
| TNFRSF5 | PN | IL10 |
| TNFRSF5 | PN | NAPK1 |
| TNFRSF5 | PN | TNFSF11 |
| TNFRSF6 | PN | NFK61 |
| Toll receptor | PN | BCL2 |
| TP53 | PN | CASP2 |
| TP53 | PN | CASP8 |
| TP53 | PN | ESR1 |
| TP53 | PN | HAPK1 |
| TP53 | PN | HDN2 |
| TP53 | PN | NET |
| TP53 | PN | PEG3 |
| TP53 | PN | PPH1D |
| TP53 | PN | PYCR1 |
| TP53 | PN | SFH |
| TP53 | PN | MT1 |
| TP73 | PN | CC [symbol] G1 |
| TP73 | PN | EDX2 |
| TRAF2 | PN | GCXR |
| TRAF2 | PN | HAP3K1 |
| TRAF2 | PN | TNFRSF1A |
| TRAF3 | PN | KFIBIA |
| TRAF3 | PN | TNFRSF5 |
| TRAF6 | PN | NAPK14 |
| TRAF6 | PN | TRAF3 |
| transcription | PN | BOL2L11 |
| factor | | |
| transcription | PN | PONG |
| factor | PN | |
| transcription | PN | PPARG |
| factor | PN | |
| transforming | PN | COKN1A |
| growth factor | PN | |
| TSG1 | PN | TSC2 |
| TTF1 | PN | TG |

TABLE 35

| Protein A | | Protein B |
|---|---|---|
| tumor necrosis | PN | BCL2L1 |
| factor | | |
| tumor necrosis | PN | HGF |
| factor | | |
| tumor necrosis | PN | [symbol] OX1 |
| factor | | |
| tumor necrosis | PN | IRS1 |
| factor | | |
| tumor necrosis | PN | LIF |
| factor | | |
| tumor necrosis | PN | NOS2A |
| factor | | |
| tumor necrosis | PN | PONC |
| factor | | |
| tumor necrosis | PN | PTGS2 |
| factor | | |
| tumor necrosis | PN | REL |
| factor | | |
| tumor necrosis | PN | S0D2 |
| factor | | |
| tumor necrosis | PN | SST |
| factor | | |
| tumor necrosis | PN | STAT6 |
| factor | | |
| tumor necrosis | PN | VCAK1 |
| factor | | |
| tumor necrosis | PN | IL13 |
| factor | | |
| UBL3 | PN | TGFB1 |
| yesopressin | PN | AVP |
| VAV1 | PN | CBL |
| VAV1 | PN | CDC42 |
| VAV1 | PN | PAK1 |
| VAV1 | PN | RAC1 |
| VAV2 | PN | CDC42 |
| VAV2 | PN | RAC1 |
| VAV3 | PN | STAT3 |
| VCAN1 | PN | PTK2 |
| VCAN1 | PN | RAC1 |
| VDR | PN | TTGAN |
| VEGF | PN | FLT1 |
| VEGF | PN | LCP1 |
| VEGF | PN | PTK2 |
| VEGF | PN | PTK2B |
| VEGF | PN | RAC1 |
| VEGF | PN | SELE |
| VIP | PN | GALGA |
| VIP | PN | GCG |
| VIP | PN | SST |
| VLDL | PN | LEP |
| voltage-dependent | PN | GN [symbol] |
| calcium channel | | |
| VTN | PN | FN1 |
| VTN | PN | SERPINE1 |
| water | PN | NPPA |
| [symbol] T1 | PN | NRDB1 |
| ZAP70 | PN | PTPK6 |
| ZAP148 | PN | TP53 |

TABLE 36

| Protein A | | Protein B |
|---|---|---|
| A2Y | PP | TGFB1 |
| ABL1 | PP | H2O2 |
| ABL1 | PP | HCK |
| ABL1 | PP | integrin |
| ABL1 | PP | HCK1 |
| ABL1 | PP | PRKCD |
| ABL1 | PP | src family |
| ABL1 | PP | TP73 |
| ACDC | PP | INS |
| acetyltransferase | PP | INS |
| ADAKTSL1 | PP | TGFB1 |
| ADCY2 | PP | FOS |
| ADCY2 | PP | MAPK3 |
| ADCY2 | PP | PONC |
| ADCYAP1 | PP | ADCY2 |
| ADCYAP1 | PP | TAC1 |
| ADORA2A | PP | MAPKS |
| ADP | PP | JUN |
| ADP | PP | MAPK8 |
| ADP | PP | P2RY1 |
| ADRBK1 | PP | RAS small monomeric |
| | | GTPase |
| AGT | PP | EDN1 |
| AGT | PP | EGF |
| AGT | PP | HRAS |
| AGT | PP | MAPK3 |
| AGT | PP | Phosphatidylinositol |
| | | 3-kinase |
| AGT | PP | PLCG1 |
| AGT | PP | TKF |
| AGT | PP | VEGF |
| AGTR1 | PP | ANG |
| AGTR1 | PP | IGF1 |
| AGTR1 | PP | SERPINE1 |
| AKR1B1 | PP | TNF |
| AKT1 | PP | ADP |
| AKT1 | PP | calcium-dependent cell |
| | | adhesion molecule |
| AKT1 | PP | catenin |
| AKT1 | PP | CDC37 |
| AKT1 | PP | CDKN1A |
| AKT1 | PP | CREB1 |
| AKT1 | PP | CSF1 |
| AKT1 | PP | CSF2 |
| AKT1 | PP | CTGF |
| AKT1 | PP | EGF |
| AKT1 | PP | estrogen |
| AKT1 | PP | F2 |
| AKT1 | PP | GSK3A |
| AKT1 | PP | HGF |
| AKT1 | PP | IFNG |
| AKT1 | PP | IGF1R |
| AKT1 | PP | IgG |
| AKT1 | PP | IL5 |
| AKT1 | PP | IL8RB |
| AKT1 | PP | [symbol] LK |
| AKT1 | PP | [symbol] RS |
| AKT1 | PP | insulins |
| AKT1 | PP | integrin |
| AKT1 | PP | JAK2 |
| AKT1 | PP | laminio |
| AKT1 | PP | LEP |
| AKT1 | PP | lipid |
| AKT1 | PP | MSK1 |
| AKT1 | PP | MYOD1 |
| AKT1 | PP | NPPA |
| AKT1 | PP | Nuclear factor NF |
| | | kappa B |
| AKT1 | PP | OR2C1 |
| AKT1 | PP | PAK1 |
| AKT1 | PP | peptide receptor, |
| | | G-protein coupled |
| AKT1 | PP | progesterone |
| AKT1 | PP | protein serine/ |
| | | theronine kinase |
| AKT1 | PP | PS |
| AKT1 | PP | PtdIns-3. 4-P 2 |
| AKT1 | PP | RAS small monomeric |
| | | GTPase |
| AKT1 | PP | RELA |

TABLE 37

| Protein A | | Protein B |
|---|---|---|
| AKT1 | PP | SLC2A1 |
| AKT1 | PP | SSTR2 |
| AKT1 | PP | THPO |
| AKT1 | PP | transmembrane receptor |
| | | protein tyrosine kinase |
| AKT1 | PP | tumor necrosis factor |
| AKT1 | PP | UCN |
| AKT1 | PP | UCN3 |
| AKT2 | PP | NYOD1 |
| ALB | PP | CAV1 |
| ALK | PP | IPN1 |
| ALOK5 | PP | TNF |
| APAF1 | PP | TP53 |
| APP | PP | IL18 |
| APP | PP | STAT3 |
| APP | PP | THF |
| APP | PP | THFRSF5 |
| AR | PP | EP300 |
| AR | PP | SRC |
| AR | PP | STAT5A |
| ARAF1 | PP | EDK1 |
| ARAF1 | PP | MAP2K1 |
| ARAF1 | PP | MAP2K2 |
| ARAF1 | PP | MAPK1 |
| ARAF1 | PP | RAS small monomeric |
| | | GTPase |
| AREG | PP | BT1 |
| ARF1P2 | PP | RAC1 |
| ARG2 | PP | TGFB1 |
| ARHGEF12 | PP | GNA13 |
| ARRB1 | PP | MAPK3 |
| ATF2 | PP | TGFB1 |
| AYP | PP | ADCY2 |
| BAX | PP | BCL2L2 |
| BAX | PP | CASP3 |
| BAX | PP | TNFRSF6 |
| BBC3 | PP | TP53 |
| BCAR1 | PP | CRK |
| BCAR1 | PP | EGF |
| BCAR1 | PP | FN1 |
| BCAR1 | PP | [symbol] NS |
| BCAR1 | PP | KRAS2 |
| BCAR1 | PP | YAPK8 |
| BCAR1 | PP | SRC |
| B-cell receptor | PP | JUN |
| BCL2 | PP | cAYP-dependent protein |
| | | kinase. |
| BCL2 | PP | Phosphatidylinositol |
| | | 3-kinase |
| BCL2 | PP | POU4F1 |
| BCL2 | PP | RPS6K |
| BCL2 | PP | STAT3 |
| BCL2 | PP | TNFRSF17 |
| BCL2 | PP | VEGF |
| BCL2L1 | PP | STAT3 |
| BCL3 | PP | CCND1 |
| BDK | PP | EGFR |
| BDK | PP | IL18 |
| BDK | PP | IL1F8 |
| BDK | PP | NGF8 |
| BDK | PP | PRKCE |
| BDK | PP | PTGS2 |
| BDK | PP | TAC1 |
| BDNF | PP | CREB1 |
| BDNF | PP | FGF2 |
| BDNF | PP | PIPN11 |
| BDNF | PP | VIP |
| BIRC3 | PP | NAPK8 |
| BKP2 | PP | BCL2L1 |
| BRAF | PP | MAP2K1 |
| BRAF | PP | MAP2K2 |
| BRAF | PP | MAPK1 |
| BRAF | PP | MAPK3 |
| BRAF | PP | NTRK1 |
| BRAF | PP | RASGRP1 |
| BRAF | PP | RPS6K |
| BRAF | PP | T-cell receptor |
| | | T3 complex |
| BRAF | PP | TERF2IP |
| BRCA1 | PP | IFNG |

TABLE 38

| Protein A | | Protein B |
|---|---|---|
| BTK | PP | LYN |
| C5R1 | PP | IL6 |
| CALCA | PP | ADCY2 |
| CALCA | PP | GCG |
| CALCA | PP | TGFB1 |
| calcium | PP | PLCG1 |
| calcium-dependent | PP | RAC1 |
| cell adhesion molecule | | |
| calmodulin | PP | EGFR |
| calpain | PP | EGF |
| CaN-kinase II | PP | EGF |
| cANP | PP | EGFR |
| cAKP-dependent | PP | ADH |
| protein kinase. | | |
| catalyst | | |
| CAPG | PP | PLCG1 |
| casein | PP | INS |
| CASP6 | PP | TP53 |
| CASP8 | PP | BAX |
| CASP8 | PP | CASP9 |
| CASP8 | PP | GYCS |
| CASP8 | PP | TNFSF6 |
| CASP9 | PP | CASP3 |
| CASP9 | PP | effector caspase |
| CASP9 | PP | PARP1 |
| CASP9 | PP | PTK2 |
| CASP9 | PP | TNFRSF6 |
| caspase | PP | CDKN1A |
| caspase | PP | MAPK3 |
| catecholamine | PP | Na+/K+ ATPase |
| CAV1 | PP | CAV2 |
| CAV1 | PP | IL6 |
| CBL | PP | EGF |
| CBL | PP | FH1 |
| CBL | PP | INS |
| CBL | PP | KDR |
| CBL | PP | Phosphatidylinositol |
| | | 3-kinase |
| CCL2 | PP | IL1A |
| CCL2 | PP | IL3 |
| CCL2 | PP | IL6 |
| CCL2 | PP | JUN |
| CCL2 | PP | MAPK3 |
| CCL2 | PP | TNF |
| CCL21 | PP | cANP |
| CCL4 | PP | TNF |
| CCL5 | PP | TFNG |
| CCL5 | PP | MAPK14 |
| CCKA2 | PP | TP53 |
| CCHD1 | PP | CDK6 |
| CCHD1 | PP | CDKN1A |
| CCHD1 | PP | EGF |
| CCHD1 | PP | ERBB2 |
| CCHD1 | PP | MAP2K1 |
| CCHD1 | PP | MAPK1 |
| CCHD1 | PP | NYC |
| CCHD1 | PP | PCAF |
| CCHD1 | PP | platelet-derived |
| | | growth factor |
| CCHD1 | PP | RAF1 |
| CCHD1 | PP | RBL2 |
| CCKH | PP | CAK complex |
| CD28 | PP | JUN |
| CD28 | PP | MAPK8 |
| CD4 | PP | IL4 |
| CD4 | PP | IL6 |
| CD44 | PP | HGF |
| CD44 | PP | TGFB1 |
| CD48 | PP | TFNG |
| CD5 | PP | NAFK8 |
| CD58 | PP | IL4 |
| CDC2 | PP | CDC25C |
| CDC25A | PP | CCNE1 |
| CDC25C | PP | NSLN |
| CDC42 | PP | F2 |
| CDC42 | PP | PTK2 |
| CDC42 | PP | RAF1 |

TABLE 39

| Protein A | | Protein B |
|---|---|---|
| CDH1 | PP | NYC |
| CDK2 | PP | OCND2 |
| CDKN1A | PP | ERCA1 |
| CDKN1A | PP | EGF |
| CDKN1A | PP | EGFR |
| CDKN1A | PP | FGF2 |
| CDKN1A | PP | HDKA10 |
| CDKN1A | PP | nap kinase |
| CDKN1A | PP | MAPK1 |
| CDKN1A | PP | HTRK1 |
| CDKN1A | PP | Nuclear factor NF |
| | | kappa 8 |
| CDKN1A | PP | PAK1 |
| CDKN1A | PP | Phosphatidylinositol |
| | | 3-kinase |
| CDKN1A | PP | PKC |
| CDKN1A | PP | PRKCA |
| CDKN1A | PP | protein serine/ |
| | | threonine kinase |
| CDKN1A | PP | protein tyrosine kinase |
| CDKN1A | PP | SLC12A9 |
| CDKN1A | PP | STAT1 |
| CDKN1A | PP | TP73 |
| CDKN1B | PP | CDKN1A |
| CDKN1B | PP | CDKN1C |
| CDKN1B | PP | JUN |
| CDKN1B | PP | NYOD1 |
| CDKN1B | PP | SP1 |
| CDKN2B | PP | TGFB1 |
| CEBPA | PP | USF1 |
| CEBFB | PP | RAS small monomeric |
| | | GTPase |
| CEBPB | PP | STAT3 |
| CEBPB | PP | transcription factor |
| CEBPG | PP | IL6 |
| choline phosphatase | PP | PRKCZ |
| choline phosphatase | PP | RHOA |
| CHUK | PP | 1KBKB |
| CHUK | PP | NF-kappa8-inducing |
| | | kinase |
| CNTF | PP | IL6 |
| CNTF | PP | NAPK1 |
| CNTF | PP | NAPK3 |
| CREB1 | PP | ADCY2 |
| CREB1 | PP | EP300 |
| CREB1 | PP | GC6 |
| CREB1 | PP | IL2 |
| CREB1 | PP | NAPK14 |
| CREB1 | PP | POKC |
| CREB1 | PP | RAC1 |
| CREB1 | PP | SRC |
| CREB1 | PP | TGFB1 |
| CRH | PP | ILG |
| CRH | PP | TNF |
| CRHR1 | PP | MAPK3 |
| CRK | PP | DOCK1 |
| CRK | PP | EGF |
| CRK | PP | NAPK1 |
| CRK | PP | NGFB |
| CRK | PP | platelet-derived |
| | | growth factor |
| CRK | PP | PKN |
| CRK | PP | RAS small monomeric |
| | | GTPase |
| CRK | PP | RPS6K |
| CRK | PP | src family |
| CRK | PP | T-cell receptor |
| | | T3 complex |
| CRKL | PP | EGF |
| CRKL | PP | RAPGEF1 |
| CSF1 | PP | CCL2 |
| CSF1 | PP | CSF2 |
| CSF1 | PP | IL6 |
| CSF1 | PP | NAPK1 |
| CSF1 | PP | STAT3 |

TABLE 40

| Protein A | | Protein B |
|---|---|---|
| CSF2 | PP | IL2 |
| CSF2 | PP | JAK2 |
| CSF2 | PP | JUN |
| CSF2 | PP | KITL6 |
| CSF2 | PP | LYN |
| CSF2 | PP | NAPK14 |
| CSF2 | PP | PTPN11 |
| CSF2 | PP | RAF1 |
| CSF2 | PP | STAT5A |
| CSF2 | PP | TNF |
| CSF2 | PP | VEGF |
| CSF3 | PP | CEBPB |
| CSF3 | PP | CISH |
| CSF3 | PP | CSF3R |
| CSF3 | PP | IL1 |
| CSF3 | PP | IL6 |
| CSF3 | PP | ITGA11 |
| CSF3 | PP | ITGAX |
| CSF3 | PP | ITGB2 |
| CSF3 | PP | JAK1 |
| CSF3 | PP | JAK2 |
| CSF3 | PP | LYN |
| CSF3 | PP | RAF1 |
| CSF3 | PP | RPS6K |
| CSF3 | PP | STAT1 |
| CSF3 | PP | TYK2 |
| CTGF | PP | FH1 |
| CTGF | PP | TGFB1 |
| CTGF | PP | VEGF |
| CTNNB1 | PP | LEF |
| CTNNB1 | PP | LEF1 |
| CX3CL1 | PP | TP53 |
| CXCL10 | PP | CXCR3 |
| CXCL5 | PP | TNF |
| CXCL9 | PP | IFNG |
| CXCR3 | PP | VAPK1 |
| cyclin | PP | E2F1 |
| CYCS | PP | CASP9 |
| CYCS | PP | effector caspase |
| CYP1A1 | PP | CYP2B6 |
| diacylglycerol | PP | PLC61 |
| DTR | PP | NAPK1 |
| E2F1 | PP | CASP9 |
| E2F1 | PP | caspase |
| E2F1 | PP | CHEK2 |
| EDN1 | PP | CKCL1 |
| EDN1 | PP | EGF |
| EDN1 | PP | HRAS |
| EDN1 | PP | YAPK3 |
| EDN1 | PP | NOS2A |
| EDN1 | PP | PLA2618 |
| EDN1 | PP | RHOA |
| EDN1 | PP | SHC1 |
| EDN1 | PP | VEGF |
| EEF2X | PP | FRAP1 |
| EGF | PP | ABL1 |
| EGF | PP | adrenoceptor |
| EGF | PP | AGTR1 |
| EGF | PP | ALB |
| EGF | PP | ARF6 |
| EGF | PP | CDC42 |
| EGF | PP | CISH |
| EGF | PP | DNN1 |
| EGF | PP | DTR |
| EGF | PP | ELK1 |
| EGF | PP | ERBE3 |
| EGF | PP | ERK activator kinase |
| EGF | PP | FN1 |
| EGF | PP | FOS |
| EGF | PP | GAB1 |
| EGF | PP | GH1 |
| EGF | PP | K202 |
| EGF | PP | IGF1 |
| EGF | PP | IL6 |
| EGF | PP | NAP2K1 |
| EGF | PP | NAPK3 |
| EGF | PP | NYC |
| EGF | PP | PTK2 |
| EGF | PP | PTPN11 |
| EGF | PP | RAF1 |

TABLE 41

| Protein A | | Protein B |
|---|---|---|
| EGF | PP | SHC1 |
| EGF | PP | SRC |
| EGF | PP | STAT3 |
| EGF | PP | TGFA |
| EGFR | PP | AREG |
| EGFR | PP | DTR |
| EGFR | PP | EPS15 |
| EGFR | PP | ERBB4 |
| EGFR | PP | FN1 |
| EGFR | PP | GAB1 |
| EGFR | PP | nap kinase |
| EGFR | PP | NAPK3 |
| EGFR | PP | NAP2 |
| EGFR | PP | Nuclear factor NF |
| | | kappa B |
| EGFR | PP | PGE2 |
| EGFR | PP | PKC |
| EGFR | PP | PLCG1 |
| EGFR | PP | PTK2 |
| EGFR | PP | PTK28 |
| EGFR | PP | SOS1 |
| EGFR | PP | SRC |
| EGFR | PP | TGFB1 |
| EGFR | PP | UBE2L3 |
| EGR1 | PP | FGF2 |
| EGR1 | PP | IGF2 |
| EGR1 | PP | IL3 |
| EGR1 | PP | interleukin-1 receptor |
| | | ligand |
| EGR1 | PP | NTSR1 |
| EGR1 | PP | PDGFA |
| EGR1 | PP | platelet-derived |
| | | growth factor |
| EGR1 | PP | RPSGK |
| EGR1 | PP | TGFB1 |
| EGR1 | PP | TNF |
| ELF3 | PP | ERBB2 |
| ELK1 | PP | JUN |
| ELK1 | PP | HAPK3 |
| ELK1 | PP | HAPK8 |
| ELK1 | PP | P2RY1 |
| ELK1 | PP | SAP kinase |
| endothelin | PP | IL6 |
| EKTPD2 | PP | EGF |
| EP300 | PP | TGFB1 |
| EPHA3 | PP | VEGF |
| EPHB1 | PP | HAPK1 |
| EPHB1 | PP | HAPK8 |
| EPX | PP | CSF3 |
| ERBB2 | PP | GRE2 |
| ERBB2 | PP | Nuclear factor NF |
| | | kappa B |
| ERB32 | PP | PTGS2 |
| ERB84 | PP | ERBB2 |
| ERK activator kinase | PP | HAPK8 |
| ESR1 | PP | AKT2 |
| ESR1 | PP | JUN |
| ESR1 | PP | HAPK1 |
| ESR1 | PP | SP1 |
| ETS1 | PP | COLIA2 |
| ETS1 | PP | ETS2 |
| ETS1 | PP | PPARA |
| ETS1 | PP | VEGF |
| ETS2 | PP | CGHD1 |
| ETY1 | PP | ERBB2 |
| F2 | PP | ICAH1 |
| F2 | PP | HAPK3 |
| F2 | PP | Phosphatidylinositol |
| | | 3-kinase |
| F2 | PP | PLAUR |
| F2 | PP | PLCG1 |
| F2 | PP | PRKCA |
| F2 | PP | protein phosphatase 1 |
| F2 | PP | PTK2B |
| F2 | PP | SHC1 |

TABLE 42

| Protein A | | Protein B |
|---|---|---|
| F2R | PP | ACRBK2 |
| F2R | PP | beta-n- |
| | | acetylglucoseninidase |
| F2R | PP | EGF |
| F2R | PP | ICAN1 |
| F2R | PP | peptide receptor, |
| | | G-protein coupled |
| F2R | PP | Phosphatidylinositol |
| | | 3-kinase |
| F2R | PP | PLAT |
| F2R | PP | RAP1B |
| F2R | PP | SELP |
| F2R | PP | SHC1 |
| F2R | PP | TXNL5 |
| F2RL1 | PP | F2R |
| F2RL1 | PP | peptide receptor, |
| | | G-protein coupled |
| F2RL1 | PP | phospholipase C |
| F3 | PP | ALB |
| F3 | PP | F2 |
| F3 | PP | IL18 |
| FCER2 | PP | IL6 |
| Ferritin | PP | IFNG |
| FGF1 | PP | Hap kinase |
| FGF1 | PP | NGFB |
| FGF1 | PP | PLK3 |
| FGF1 | PP | RPS6K |
| FGF18 | PP | ERK activator kinase |
| FGF2 | PP | BCL2L1 |
| FGF2 | PP | CD44 |
| FGF2 | PP | JUN |
| FGF2 | PP | NPPA |
| FGF2 | PP | PLOG1 |
| FGF2 | PP | PRL |
| FGF2 | PP | PTGS2 |
| FGF2 | PP | PTPH11 |
| FGF2 | PP | TGFB1 |
| FGF2 | PP | VEGF |
| FGF7 | PP | HAPK1 |
| fibroblast growth | PP | TNF |
| factor | | |
| FN1 | PP | AGT |
| FN1 | PP | EDN1 |
| FN1 | PP | HAP2K1 |
| FN1 | PP | HAPK1 |
| FN1 | PP | HAP9 |
| FN1 | PP | PTK2 |
| FN1 | PP | RHOA |
| FN1 | PP | VEGF |
| FOS | PP | AGT |
| FOS | PP | BDNF |
| FOS | PP | CREB1 |
| FOS | PP | ELK1 |
| FOS | PP | JUNB |
| FOS | PP | VAPK8 |
| FOS | PP | ODC1 |
| FOS | PP | RAF1 |
| FOS | PP | RPSGKB1 |
| FOS | PP | SRG |
| FOS | PP | STAT3 |
| FOSB | PP | JUN |
| FYN | PP | ELK1 |
| FYN | PP | PLCG1 |
| g protein | PP | EGFR |
| GA17 | PP | TFNG |
| GAB1 | PP | HGF |
| GAB1 | PP | nap kinase |
| GAB1 | PP | Phosphatidylinositol |
| | | 3-kinase |
| GAB1 | PP | platelet-derived |
| | | growth factor |
| GADD45A | PP | COKN1A |
| GADD45A | PP | NAPK8 |
| GAP | PP | NAPK1 |
| GAS | PP | THF |

TABLE 43

| Protein A | | Protein B |
|---|---|---|
| GCG | PP | ADCY2 |
| GCG | PP | NAP2K1 |
| GCG | PP | NAPK1 |
| GCG | PP | NAPK3 |
| GCG | PP | PONC |
| GDNF | PP | HAPK1 |
| GEF | PP | RAC1 |
| GH1 | PP | CEBP3 |
| GH1 | PP | GNRH1 |
| GH1 | PP | IRS1 |
| GH1 | PP | JAK2 |
| GJAI | PP | NAPK3 |
| GLI | PP | GLI2 |
| glucose | PP | PPARGC1A |
| glutamate receptor | PP | NAPK1 |
| GNA12 | PP | NAPK3 |
| GNAO | PP | RHOA |
| GNRH1 | PP | ADCYAP1 |
| GNRH1 | PP | EGFR |
| GNRHR | PP | EGFR |
| GNRHR | PP | NAPK1 |
| GRB2 | PP | BCR |
| GRB2 | PP | EGF |
| GRB2 | PP | LPA |
| GRB2 | PP | SHC1 |
| Group] cetabotropic | PP | NAPK3 |
| glutamate receptor | PP | JUN |
| growth factor | | |
| receptor | PP | BCL2L11 |
| GSK3B | | |
| GSK3B | PP | BDNF |
| GSK3B | PP | NAP2K1 |
| GTPase | PP | GNAO |
| guanine nucleotide | PP | RAC1 |
| exchange factor | | |
| GYPC | PP | IL6 |
| H+/K+ ATPase | PP | AKT1 |
| H2O2 | PP | POGFB |
| H2O2 | PP | PLGG1 |
| HAND1 | PP | IFNG |
| HGF | PP | IL6 |
| HGF | PP | NGFB |
| HGF | PP | NOS2A |
| HGF | PP | PLAU |
| HGF | PP | PLAUR |
| HGF | PP | PTPH11 |
| HGF | PP | SP1 |
| HGF | PP | SRC |
| HGF | PP | VEGF |
| HGS | PP | EGF |
| HIFIA | PP | GSK38 |
| HLA-A | PP | IFNG |
| HNOX1 | PP | IL6 |
| HNOX1 | PP | NAPK14 |
| HNOX1 | PP | NAPK3 |
| HRAS | PP | EGFR |
| HRAS | PP | KRT18 |
| HRAS | PP | PLC61 |
| HSPA4 | PP | HSPCA |
| HSPB2 | PP | TNF |
| HSPCA | PP | NOS3 |
| HSPCA | PP | STAT3 |
| HSFCA | PP | VEGF |
| HSPE1 | PP | HSPD1 |
| HTATIP | PP | IL6 |
| ICAN1 | PP | CCL5 |
| ICAN1 | PP | FOS |
| ICAN1 | PP | IL2 |
| ICAN1 | PP | HAPK8 |
| ICAN1 | PP | STAT1 |
| IER2 | PP | JUN |
| IER2 | PP | INF |

TABLE 44

| Protein A | | Protein B |
|---|---|---|
| IFNG | PP | B2H |
| IFNG | PP | B7H3 |
| IFNG | PP | GASP1 |
| IFNG | PP | CCR5 |
| IFNG | PP | CCR6 |
| IFNG | PP | CD69 |
| IFNG | PP | CD86 |
| IFNG | PP | COK5R1 |
| IFNG | PP | CEACAY5 |
| IFNG | PP | CK3CL1 |
| IFNG | PP | CKCL16 |
| IFNG | PP | DPP4 |
| IFNG | PP | FCGR1A |
| IFNG | PP | HLA-B |
| IFNG | PP | HLA-E |
| IFNG | PP | HSPA4 |
| IFNG | PP | IL15 |
| IFNG | PP | IL-18 receptor |
| IFNG | PP | IL1F8 |
| IFNG | PP | IL2RB |
| IFNG | PP | IRF1 |
| IFNG | PP | JAK2 |
| IFNG | PP | LTA |
| IFNG | PP | LY96 |
| IFNG | PP | neopterin |
| IFNG | PP | NOS2A |
| IFNG | PP | OAS1 |
| IFNG | PP | PIH1 |
| IFNG | PP | PLA2618 |
| IFNG | PP | RA |
| IFNG | PP | RPS6K |
| IFNG | PP | SELL |
| IFNG | PP | SFTPA1 |
| IFNG | PP | SNN2 |
| IFNG | PP | STAT3 |
| IFNG | PP | STAT5A |
| IFNG | PP | TACR1 |
| IFNG | PP | TBK21 |
| IFNG | PP | TKFRSF6 |
| IFKG | PP | TRIY8 |
| IGBPI | PP | CCND1 |
| IGF1 | PP | BCL2 |
| IGF1 | PP | BDNF |
| IGF1 | PP | CCNA2 |
| IGF1 | PP | CRK |
| IGF1 | PP | EGFR |
| IGF1 | PP | FOS |
| IGF1 | PP | VAPK3 |
| IGF1 | PP | OXT |
| IGF1 | PP | POYC |
| IGF1 | PP | PRL |
| IGF1 | PP | PTGS2 |
| IGF1 | PP | PTK2 |
| IGF1 | PP | PTPN11 |
| IGF1 | PP | RAF1 |
| IGF1 | PP | SHC1 |
| IGF1 | PP | SRC |
| IGF1R | PP | IGF1 |
| IGFBP3 | PP | IP53 |
| IHH | PP | KAPK3 |
| IkappaB kinase | PP | CHUK |
| IL1 | PP | ECR1 |
| IL1 | PP | IFNG |
| IL1 | PP | IL6 |
| IL1 | PP | YAPK1 |
| IL10 | PP | POYC |
| IL13 | PP | IL2 |
| IL13 | PP | IL3 |
| IL13 | PP | IL5 |
| IL17 | PP | TNF |
| IL18 | PP | IFNG |

TABLE 45

| Protein A | | Protein B |
|---|---|---|
| IL1A | PP | CGL5 |
| IL1A | PP | CSF2 |
| IL1A | PP | CXCL10 |
| IL1A | PP | F2 |
| IL1A | PP | FNI |
| IL1A | PP | HGF |
| IL1A | PP | IL1B |
| IL1A | PP | IL1F8 |
| IL1A | PP | NFKBIA |
| IL1A | PP | NGFB |
| IL1B | PP | CEBPB |
| IL1B | PP | CSF1 |
| IL1B | PP | CSF2 |
| IL1B | PP | EDNI |
| IL1B | PP | HGF |
| IL1B | PP | HSPA4 |
| IL1B | PP | IL6 |
| IL1B | PP | KIILG |
| IL1B | PP | NAPK1 |
| IL1B | PP | NAPK14 |
| IL1B | PP | NAPK3 |
| IL1B | PP | NAPK8 |
| IL1B | PP | TAC1 |
| IL1B | PP | TNFRSF6 |
| IL1F8 | PP | AKT1 |
| IL1F8 | PP | CD44 |
| IL1F8 | PP | EGRI |
| IL1F8 | PP | FOS |
| IL1F8 | PP | NAPK14 |
| IL1F8 | PP | TNFRSF5 |
| IL1F8 | PP | VCAN1 |
| IL2 | PP | AKTI |
| IL2 | PP | CD4 |
| IL2 | PP | CO44 |
| IL2 | PP | CSF1 |
| IL2 | PP | GRB2 |
| IL2 | PP | HRAS |
| IL2 | PP | IL1B |
| IL2 | PP | JUN |
| IL2 | PP | KITLG |
| IL2 | PP | NAPK1 |
| IL2 | PP | NAPK3 |
| IL2 | PP | PLAUR |
| IL2 | PP | PLCG1 |
| IL2 | PP | PONC |
| IL2 | PP | RAF1 |
| IL2 | PP | SHG1 |
| IL2 | PP | STAT5A |
| IL2 | PP | TNFRSF5 |
| IL2 | PP | TNFSF6 |
| IL24 | PP | IL20RB |
| IL3 | PP | AKTI |
| IL3 | PP | CBL |
| IL3 | PP | CSF2 |
| IL3 | PP | ILIA |
| IL3 | PP | ILIB |
| IL3 | PP | IL6 |
| IL3 | PP | JAK2 |
| IL3 | PP | LYN |
| IL3 | PP | NAPK1 |
| IL3 | PP | NAPK8 |
| IL3 | PP | PTPN11 |
| IL3 | PP | SHG1 |
| IL4 | PP | IRS1 |
| IL4 | PP | NAPK14 |
| IL4 | PP | PLCG1 |
| IL4 | PP | TNFRSF5 |
| IL4 | PP | VCAN1 |
| IL5 | PP | VCAN1 |
| IL5 | PP | ILIB |
| IL5 | PP | JAK2 |
| IL5 | PP | JUN |
| IL5 | PP | NAPK3 |
| IL5 | PP | SHC1 |

TABLE 46

| Protein A | | Protein B |
|---|---|---|
| IL6 | PP | A2H |
| IL6 | PP | ACP1 |
| IL6 | PP | AGT |
| IL6 | PP | AKT1 |
| IL6 | PP | APP |
| IL6 | PP | AREG |
| IL6 | PP | cAMP |
| IL6 | PP | CD14 |
| IL6 | PP | CEBPD |
| IL6 | PP | CRP |
| IL6 | PP | CSF2 |
| IL6 | PP | EBP |
| IL6 | PP | EDN1 |
| IL6 | PP | ELA2 |
| IL6 | PP | F3 |
| IL6 | PP | FCGR3A |
| IL6 | PP | FGF7 |
| IL6 | PP | FH1 |
| IL6 | PP | FOS |
| IL6 | PP | GZMA |
| IL6 | PP | H2O2 |
| IL6 | PP | hemoglobin |
| IL6 | PP | histamine |
| IL6 | PP | HSPB2 |
| IL6 | PP | ICAN1 |
| IL6 | PP | TGF1 |
| IL6 | PP | IL1A |
| IL6 | PP | IL2 |
| IL6 | PP | IL6R |
| IL6 | PP | IL8 |
| IL6 | PP | IILH4 |
| IL6 | PP | KIT |
| IL6 | PP | lactate |
| IL6 | PP | LIF |
| IL6 | PP | LTB4 |
| IL6 | PP | MAPK3 |
| IL6 | PP | YHC class II complex |
| IL6 | PP | YHC class II protein |
| IL6 | PP | NOS2A |
| IL6 | PP | PGE1 |
| IL6 | PP | PKG |
| IL6 | PP | PLA2G10 |
| IL6 | PP | PHA |
| IL6 | PP | PTGS1 |
| IL6 | PP | PTGS2 |
| IL6 | PP | PTPRC |
| IL6 | PP | RAS small monomeric |
| | | GTPase |
| IL6 | PP | SLC12A4 |
| IL6 | PP | STAT1 |
| IL6 | PP | TAC1 |
| IL6 | PP | TNFRSF1A |
| IL6 | PP | TNFRSF5 |
| IL6 | PP | TNFSF11 |
| IL6 | PP | TRAF3 |
| IL6 | PP | TYK2 |
| IL6 | PP | VEGF |
| IL6ST | PP | CDKN1A |
| IL8 | PP | CEBPB |
| IL8 | PP | CSF3 |
| IL8 | PP | EDN1 |
| IL8 | PP | EGFR |
| IL8 | PP | FGF2 |
| IL8 | PP | IL1A |
| IL8 | PP | MAPK8 |
| IL8 | PP | POYC |
| IL8 | PP | TAC1 |
| IL8 | PP | TGFA |
| IL8 | PP | TNFSF6 |
| IL8 | PP | VEGF |
| IL8RA | PP | IL8RB |
| IL8RB | PP | CXCL1 |
| ILF | PP | IL8 |
| ILK | PP | TGFB1 |
| I [symbol] HBA | PP | TGFB1 |
| inositol lipids | PP | AXT1 |

TABLE 47

| Protein A | | Protein B |
|---|---|---|
| INS | PP | ADRB2 |
| INS | PP | AIB |
| INS | PP | ANXA1 |
| INS | PP | ARNT |
| INS | PP | atypical protein |
| | | kinase C |
| INS | PP | calmodulin |
| INS | PP | CDC42 |
| INS | PP | CREB1 |
| INS | PP | CSF1 |
| INS | PP | EDN1 |
| INS | PP | EGF |
| INS | PP | EGR1 |
| INS | PP | estradiol |
| INS | PP | FGF1 |
| INS | PP | fibroblast growth factor |
| INS | PP | GAS |
| INS | PP | GCXR |
| INS | PP | GRB10 |
| INS | PP | GRB2 |
| INS | PP | IGF1R |
| INS | PP | IGFBP5 |
| INS | PP | IL1RN |
| INS | PP | IP3 |
| INS | PP | IPF1 |
| INS | PP | JAK2 |
| INS | PP | KLK3 |
| INS | PP | map kinase |
| INS | PP | MAPK1 |
| INS | PP | HIF |
| INS | PP | HEK6 |
| INS | PP | HGF8 |
| INS | PP | Nuclear factor NF |
| | | kappa B |
| INS | PP | ODC1 |
| INS | PP | OKT |
| INS | PP | PDYN |
| INS | PP | phospholipase C |
| INS | PP | PKC |
| INS | PP | PKLR |
| INS | PP | platelet-derived |
| | | growth facor |
| INS | PP | P [symbol] CH |
| INS | PP | PONG |
| INS | PP | PRKCZ |
| INS | PP | protein phosphatase |
| INS | PP | protein tyrosine kinase |
| INS | PP | PTGDS |
| INS | PP | PTHLH |
| INS | PP | PTPN11 |
| INS | PP | pyruvate dehydrogenase |
| INS | PP | RA |
| INS | PP | RAS small monomeric |
| | | GTPase |
| INS | PP | RPS6KA3 |
| INS | PP | SOS1 |
| INS | PP | SRC |
| INS | PP | src family |
| INS | PP | TRH |
| INS | PP | TUB |
| INS | PP | VEGF |
| insulin receptor | PP | GAB1 |
| insulin receptor | PP | SOS1 |
| insulin receptor | PP | PRKCA |
| integrin | PP | FLT1 |
| integrin | PP | PDGFB |
| integrin | PP | RHOA |
| interleukin IL12 | PP | IFNG |
| interleukin IL12 | PP | TNF |
| interleukin IL12 | PP | IFNG |
| receptor | | |
| interleukin IL2 | PP | BCL2 |
| receptor | | |
| interleukin IL2 | PP | IFNG |
| receptor | | |
| interleukin IL2 | PP | PTK2B |
| receptor | | |
| IRF1 | PP | STAT1 |
| IRF7 | PP | MAPK8 |
| IRS1 | PP | PTK2 |

TABLE 48

| Protein A | | Protein B |
|---|---|---|
| ITK | PP | TGFB1 |
| JAK1 | PP | IFNG |
| JAK2 | PP | EGFR |
| JAK2 | PP | IGF1 |
| JUN | PP | JUN |
| JUN | PP | PRKCE |
| JUN | PP | ABCB1 |
| JUN | PP | ABL1 |
| JUN | PP | AKT1 |
| JUN | PP | ATF2 |
| JUN | PP | ATF3 |
| JUN | PP | CANK2A |
| JUN | PP | caspase |
| JUN | PP | CNTF |
| JUN | PP | EDN1 |
| JUN | PP | EGFR |
| JUN | PP | FH1 |
| JUN | PP | IL6 |
| JUN | PP | IL8 |
| JUN | PP | map kinase |
| JUN | PP | MAP2K4 |
| JUN | PP | MAP3K1 |
| JUN | PP | MAPK14 |
| JUN | PP | MET |
| JUN | PP | MITF |
| JUN | PP | MAP9 |
| JUN | PP | NFAT |
| JUN | PP | NFKB1 |
| JUN | PP | Nuclear factor NF |
| | | kappa B |
| JUN | PP | ODC1 |
| JUN | PP | PGE2 |
| JUN | PP | platelet-derived |
| | | growth factor |
| JUN | PP | protein serine/ |
| | | threonine kinase |
| JUN | PP | protein tyrosine kinase |
| JUN | PP | RAF1 |
| JUN | PP | replication factor C |
| JUN | PP | RIPK2 |
| JUN | PP | RPS6K |
| JUN | PP | SP1 |
| JUN | PP | SPP1 |
| JUN | PP | TNF |
| JUN | PP | TNFRSF6 |
| JUN | PP | tumor necrosis factor |
| JUN | PP | VEGF |
| JUNB | PP | FOSL1 |
| JUND | PP | YAPK3 |
| KDR | PP | IFNG |
| KDR | PP | PLOG1 |
| KITLG | PP | HGF |
| KITLG | PP | IL1A |
| KITLG | PP | IL3 |
| KITLG | PP | IL4 |
| LC [symbol] | PP | JUN |
| LDL | PP | TNF |
| LDLR | PP | APOE |
| LEF | PP | CCND1 |
| LEP | PP | CCK |
| LEP | PP | EDN1 |
| LEP | PP | IL16 |
| LEP | PP | IL1F8 |
| LEP | PP | IL6 |
| LEP | PP | LIF |
| LEP | PP | MAPK8 |
| LEP | PP | PRL |
| LIF | PP | CEBP8 |
| LIF | PP | KITLG |
| LIF | PP | PRKCD |
| LIF | PP | STAT3 |
| LIF | PP | TNF |
| LIP | PP | PTPN11 |
| LPA | PP | EGF |
| LPA | PP | IGF1 |
| LPA | PP | IL6 |
| LPA | PP | RHOA |
| LPA | PP | SERPINE1 |
| LPS | PP | MAPK1 |
| LPS | PP | MAPK3 |

TABLE 49

| Protein A | | Protein B |
|---|---|---|
| LRPAP1 | PP | MAPK1 |
| LTA | PP | TNF |
| LTB | PP | MAPK1 |
| LTB | PP | TNF |
| LYN | PP | B-cell receptor |
| LYN | PP | IL5 |
| LYN | PP | platelet-derived |
| | | growth factor |
| LYN | PP | protein tyrosine kinase |
| MAD | PP | TGFB1 |
| map kinase | PP | EGR1 |
| map kinase | PP | TNF |
| map kinase | PP | IFNG |
| MAP2K1 | PP | IL2 |
| MAP2K1 | PP | IL6 |
| MAP2K1 | PP | JUN |
| MAP2K1 | PP | PTK2 |
| MAP2K6 | PP | JUN |
| MAP2K6 | PP | MAPK3 |
| MAP2K6 | PP | MAPK8 |
| MAP3K1 | PP | MAPK2K1 |
| MAP3K1 | PP | MAPK3 |
| MAP3K1 | PP | TP53 |
| MAP3K11 | PP | MAPK1 |
| MAP3K5 | PP | MAPK8 |
| MAP3K8 | PP | MAPK8 |
| MAPK1 | PP | ADAN17 |
| MAPK1 | PP | ADCY2 |
| MAPK1 | PP | ADP |
| MAPK1 | PP | AGTR1 |
| MAPK1 | PP | ALON12 |
| MAPK1 | PP | CaS-kinase II |
| MAPK1 | PP | DCL5 |
| MAPK1 | PP | CDKN1B |
| MAPK1 | PP | CFLAR |
| MAPK1 | PP | CKCL12 |
| MAPK1 | PP | ELK1 |
| MAPK1 | PP | FPR1 |
| MAPK1 | PP | GJA1 |
| MAPK1 | PP | GRB2 |
| MAPK1 | PP | MAP kinase |
| MAPK1 | PP | MAPK9 |
| MAPK1 | PP | MP3 |
| MAPK1 | PP | MSK1 |
| MAPK1 | PP | NYDA receptor |
| MAPK1 | PP | FDPK1 |
| MAPK1 | PP | PKG |
| MAPK1 | PP | PPARA |
| MAPK1 | PP | PRKCZ |
| MAPK1 | PP | protein phosphatase |
| MAPK1 | PP | PTK2B |
| MAPK1 | PP | RPS6KA2 |
| MAPK1 | PP | RPS5KA4 |
| MAPK1 | PP | SSTR4 |
| MAPK1 | PP | TERF2IP |
| MAPK1 | PP | TLR4 |
| MAPK1 | PP | TNFSF11 |
| MAPK1 | PP | tumor necrosis factor |
| MAPK14 | PP | AGT |
| MAPK14 | PP | CSF1 |
| MAPK14 | PP | EDN1 |
| MAPK14 | PP | ELA2 |
| MAPK14 | PP | TCAN1 |
| MAPK14 | PP | IL2 |
| MAPK14 | PP | K [symbol] LG |
| MAPK14 | PP | NGFB |
| MAPK14 | PP | PDGFRA |
| MAPK14 | PP | STAT1 |
| MAPK14 | PP | VEGF |

TABLE 50

| Protein A | | Protein B |
|---|---|---|
| MAPK3 | PP | AGTR1 |
| MAPK3 | PP | CSF2 |
| MAPK3 | PP | EGR1 |
| MAPK3 | PP | EIF354 |
| MAPK3 | PP | glutamate receptor |
| MAPK3 | PP | H2O2 |
| MAPK3 | PP | HTATIP |
| MAPK3 | PP | IL6R |
| MAPK3 | PP | IL8R8 |
| MAPK3 | PP | insulin receptor |
| MAPK3 | PP | LRPAP1 |
| MAPK3 | PP | NYP3 |
| MAPK3 | PP | NPPA |
| MAPK3 | PP | Nuclear factor NF |
| | | kappa 8 |
| MAPK3 | PP | PDEGH |
| MAPK3 | PP | PKA |
| MAPK3 | PP | PLA2GIB |
| MAPK3 | PP | prostaglandin |
| MAPK3 | PP | protein serine/ |
| | | threonine kinase |
| MAPK3 | PP | RAF1 |
| MAPK3 | PP | RAPGEF1 |
| MAPK3 | PP | RASGRP1 |
| MAPK3 | PP | receptor tyrosine kinase |
| MAPK3 | PP | RPS6KB1 |
| MAPK3 | PP | TNF |
| MAPK3 | PP | TNFRSF5 |
| MAPK3 | PP | transcription factor |
| MAPK3 | PP | tranmembrane receptor |
| | | protein tyrosine kinase |
| MAPK3 | PP | VEGF |
| MAPK8 | PP | APP |
| MAPK8 | PP | B-cell receptor |
| MAPK8 | PP | CASP3 |
| MAPK8 | PP | CASP8 |
| MAPK8 | PP | CASP9 |
| MAPK8 | PP | CCL5 |
| MAPK8 | PP | CYCS |
| MAPK8 | PP | DUSP22 |
| MAPK8 | PP | EGFR |
| MAPK8 | PP | EPHA4 |
| MAPK8 | PP | EPX |
| MAPK8 | PP | F2 |
| MAPK8 | PP | FADD |
| MAPK8 | PP | HYOX1 |
| MAPK8 | PP | HTATIP |
| MAPK8 | PP | IL1F8 |
| MAPK8 | PP | LTB |
| MAPK8 | PP | nap kinase |
| MAPK8 | PP | MAP2K7 |
| MAPK8 | PP | MAP3K1 |
| MAPK8 | PP | MAP3K11 |
| MAPK8 | PP | MAP3K2 |
| MAPK8 | PP | MAP3K3 |
| MAPK8 | PP | MAP3K4 |
| MAPK8 | PP | NEDD9 |
| MAPK8 | PP | PGE2 |
| MAPK8 | PP | PIAS1 |
| MAPK8 | PP | platelet-derived |
| | | growth factor |
| MAPK8 | PP | PTGS2 |
| MAPK8 | PP | PTK23 |
| MAPK8 | PP | RAS small monomeric |
| | | GTPase |
| MAPK8 | PP | replication factor C |
| MAPK8 | PP | RHOB |
| MAPK8 | PP | src family |
| MAPK8 | PP | telomerase |
| MAPK8 | PP | transcription factor |
| MAPK8 | PP | VEGF |
| MBP | PP | INS |
| MBP | PP | TGFB1 |
| MCL1 | PP | EGFR |
| MDH2 | PP | ligase |
| MDK2 | PP | RAS small monomeric |
| | | GTPase |

TABLE 51

| Protein A | | Protein B |
|---|---|---|
| MDP | PP | F2R |
| MDP | PP | IFKG |
| MDP | PP | IL6 |
| MDP | PP | TNF |
| MDP | PP | TNF |
| MHP1 | PP | IL6 |
| MHP2 | PP | BS6 |
| MHP2 | PP | EPHA1 |
| MHP2 | PP | FGF2 |
| MHP2 | PP | IL1F8 |
| MHP2 | PP | NKP13 |
| MHP2 | PP | NKP14 |
| MHP2 | PP | NKP9 |
| MHP2 | PP | VEGF |
| MHP7 | PP | EGFR |
| MHP9 | PP | EGF |
| MHP9 | PP | IL8 |
| MHP9 | PP | TGF31 |
| MHP9 | PP | TNF |
| MHP9 | PP | VEGF |
| MPO | PP | CSF3 |
| MYC | PP | PRKCE |
| MYC | PP | RAF1 |
| MYC | PP | TNF |
| MYC | PP | TNFSF6 |
| NBS1 | PP | TP53 |
| NF1 | PP | GTPase |
| NFKB1 | PP | CCHD1 |
| NGFB | PP | BONF |
| NGFB | PP | CREB1 |
| NGFB | PP | FOS |
| NGFB | PP | GAB1 |
| NGFB | PP | GSK3B |
| NGFB | PP | IL3 |
| NGFB | PP | IL6 |
| NGFB | PP | NAPK1 |
| NGFB | PP | NAPK3 |
| NGFB | PP | PRKCE |
| NGFB | PP | PTGS2 |
| NGFB | PP | TAC1 |
| NGFR | PP | NAPK3 |
| NGFR | PP | TP53 |
| nitric oxide synthase | PP | IFNG |
| NO | PP | NAPK1 |
| NOS2A | PP | JAK2 |
| NOS2A | PP | NAPK3 |
| NOS3 | PP | AKT1 |
| NPPA | PP | PRKCA |
| NR1T2 | PP | CYP3A4 |
| NRP1 | PP | EGFR |
| NTRK1 | PP | NAPK3 |
| NTRK3 | PP | NAPK3 |
| Nuclear factor NF | PP | CKCL1 |
| kappa B | | |
| ODC1 | PP | RHO4 |
| OSH | PP | NAPK3 |
| OXT | PP | PTES2 |
| P2Y receptor | PP | NAPK3 |
| PAK1 | PP | TNF |
| PAKR | PP | F2R |
| PAKR | PP | TNF |
| PC4 | PP | PSNO9 |
| PCNA | PP | TP53 |
| PDGFB | PP | platelet-derived |
| | | growth factor |
| PDPK1 | PP | RAS small monomeric |
| | | GTPase |
| PDPK1 | PP | Rho small monomeric |
| | | GTPase |
| peptide receptor, | PP | RHOA |
| G-protein coupled | | |
| PGE1 | PP | YAPK8 |
| PGE2 | PP | CEBPB |
| PGE2 | PP | ILG |
| Phosphatidylinositol | PP | E [symbol] F4EBP1 |
| 3-kinase | | |
| Phosphatidylinositol | PP | PLA2G13 |
| 3-kinase | | |
| Phosphatidylinositol | PP | RAC1 |
| 3-kinase | | |

TABLE 52

| Protein A | | Protein B |
|---|---|---|
| phospholipase | PP | TNF |
| phospholipase C | PP | PLCG1 |
| PKA | PP | TGFB1 |
| PKC | PP | EGR1 |
| PKC | PP | ETS1 |
| PKC | PP | F2R |
| PKC | PP | PRKCABP |
| PKC | PP | RHOA |
| PLA2G1B | PP | IL2 |
| PLA2G1B | PP | IL6 |
| PLA2G1B | PP | NAPK1 |
| PLA2G1B | PP | NAPK14 |
| PLA2G1B | PP | PTGS2 |
| platelet-derived | PP | AKT1 |
| growth factor | | |
| platelet-derived | PP | EIF4E |
| growth factor | | |
| platelet-derived | PP | LPA |
| growth factor | | |
| platelet-derived | PP | SERPIKE1 |
| growth factor | | |
| PLAUR | PP | integrin |
| PLAUR | PP | PLAU |
| PLAUR | PP | PLG |
| PLAUR | PP | thronbin |
| PLCG1 | PP | choline phosphatase |
| PLCG1 | PP | fibroblast growth factor |
| PLCG1 | PP | growth factor receptor |
| PLCG1 | PP | HGF |
| PLCG1 | PP | inositol phosphate |
| PLCG1 | PP | inositol phosphates |
| PLCG1 | PP | INS |
| PLCG1 | PP | Phosphatidylinositol |
| | | 3-kinase |
| PLCG1 | PP | protein tyrosine kinase |
| PLCG1 | PP | src family |
| PLCG1 | PP | T-cell receptor |
| | | T3 complex |
| PLCG1 | PP | TERF2TP |
| PLG | PP | fibrin(ogen) |
| PNA | PP | TGFB1 |
| POYC | PP | CXCL1 |
| POYC | PP | FN1 |
| POYC | PP | FOS |
| POYC | PP | GH1 |
| POYC | PP | IL6 |
| PPARA | PP | CEBPG |
| PRKCA | PP | INS |
| PRKCA | PP | RAF1 |
| PRKCD | PP | [symbol] FNA1 |
| PRKCD | PP | YAPK14 |
| PRKCD | PP | RAF1 |
| PRKCD | PP | TGF31 |
| PRKCE | PP | EGF |
| PRKCE | PP | Nuclear factor NF |
| | | kappa B |
| PRKCE | PP | PKC |
| PRKCE | PP | PRKCZ |
| PRKCZ | PP | IL1 |
| PRKCZ | PP | map kinase |
| PRKCZ | PP | Nuclear factor NF |
| | | kappa B |
| PRKCZ | PP | Phosphatidylinositol |
| | | 3-kinase |
| PRKCZ | PP | RPS6K |
| PRL | PP | IL18 |
| PRL | PP | INS |
| PRL | PP | YAPK1 |
| PRL | PP | ODC1 |
| PRL | PP | STAT1 |
| PRL | PP | TAC1 |
| PROCR | PP | THBD |
| progesterone | PP | NAPK1 |
| PROS1 | PP | EL6R |
| protein phosphatase | PP | YAPK8 |
| protein serine/ | PP | NAPK1 |
| threonine kinase | | |

TABLE 53

| Protein A | | Protein B |
|---|---|---|
| protein tyrosine | PP | GAB1 |
| kinase | | |
| protein tyrosine | PP | NAPK3 |
| kinase | | |
| protein tyrosine | PP | RHOA |
| kinase | | |
| protein tyrosine | PP | TGFB1 |
| kinase | | |
| PSYD9 | PP | COKN1A |
| PTEN | PP | CCND3 |
| PTGIS | PP | ADCY2 |
| PTGIS | PP | thromboxane A2 |
| PTGIS | PP | VE [symbol] F |
| PTGS2 | PP | EGFR |
| PTGS2 | PP | HSPA4 |
| PTGS2 | PP | YAP2K1 |
| PTGS2 | PP | REN |
| PTH | PP | voltage-gated calcium |
| | | channel |
| PTHLH | PP | IL6 |
| PTK2 | PP | ARHGEF12 |
| PTK2 | PP | GRB2 |
| PTK2B | PP | AGT |
| PTK2B | PP | COKN1A |
| PTK2B | PP | RPSGKB1 |
| PTK2B | PP | SYK |
| PTPH11 | PP | IL6 |
| RAC1 | PP | ARF1 |
| RAC1 | PP | ARF6 |
| RAC1 | PP | CO44 |
| RAC1 | PP | EGFR |
| RAC1 | PP | FGF2 |
| RAC1 | PP | GDP |
| RAC1 | PP | IL1 |
| RAC1 | PP | KITLG |
| RAC1 | PP | RASGRP1 |
| RAC1 | PP | TNFSF6 |
| RAC1 | PP | WASF2 |
| RAF1 | PP | COKN1A |
| RAF1 | PP | EGFR |
| RAF1 | PP | ELK1 |
| RAF1 | PP | HSPCA |
| RAF1 | PP | HAP [symbol] K1 |
| RAF1 | PP | SRC |
| RAF1 | PP | TGFB1 |
| RAPGEF1 | PP | NAPK1 |
| RARA | PP | [symbol] L3 |
| RARA | PP | TGFB1 |
| RAS small monomeric | PP | CDKN1A |
| GTPase | | |
| RAS small monomeric | PP | EGR1 |
| GTPase | | |
| RAS small monomeric | PP | NAPK3 |
| GTPase | | |
| RASGRP1 | PP | NAPK1 |
| RB1 | PP | HGF |
| RB1 | PP | NYC |
| RBL1 | PP | E2F4 |
| RELA | PP | TNFRSF6 |
| replication factor C | PP | NAPK1 |
| Rho kinase | PP | RHOA |
| RHOA | PP | Glass A G-protein |
| | | coupled receptor |
| RHOA | PP | EGR1 |
| RHOA | PP | GNA12 |
| RHOA | PP | GNA13 |
| RHOA | PP | NCF2L |
| RHOA | PP | PPPIR12A |
| RHOC | PP | NAPK3 |
| RHOC | PP | RAC1 |
| ROCK1 | PP | RHOA |
| RPS6K | PP | COKN2A |
| RPS6K | PP | ELK1 |
| RPS6K | PP | ERBB2 |
| RPS6K | PP | NAPK3 |
| RPS6K | PP | src family |

TABLE 54

| Protein A | | Protein B |
|---|---|---|
| RPS5KA2 | PP | HAPK3 |
| RPS6KB1 | PP | CSF2 |
| RPS6KB1 | PP | [symbol] GF1 |
| RPS6KB1 | PP | [symbol] AK |
| RP56KB1 | PP | SRC |
| RRAS | PP | BCAR1 |
| RXRA | PP | TNF |
| SAA2 | PP | TNF |
| SAP kinase | PP | [symbol] N |
| SAP kinase | PP | NAPK1 |
| SCYL1 | PP | TNF |
| SERPINE1 | PP | F3 |
| SERPINE1 | PP | IL6 |
| SFTPA1 | PP | IL6 |
| SHC1 | PP | IL6 |
| SHC1 | PP | [symbol] S |
| SHC1 | PP | NGFB |
| SHC1 | PP | PLDG1 |
| SIAH1 | PP | CDKN1A |
| signal peptide | PP | CRK |
| peptidase | | |
| small monomeric | PP | HAPK3 |
| GTPase | | |
| SOSI | PP | HAPK8 |
| SPI | PP | SERPINE1 |
| SP [symbol] B | PP | IFR6 |
| SRC | PP | CBL |
| SRC | PP | CCND1 |
| SRC | PP | CTGF |
| SRC | PP | ERBB2 |
| SRC | PP | HIF1A |
| SRC | PP | ICAN1 |
| SRC | PP | NYG |
| SRC | PP | NPPA |
| SRC | PP | PTK2B |
| SRC | PP | SHC1 |
| src family | PP | YAPK1 |
| src family | PP | YAPK3 |
| src family | PP | PTK2 |
| src family | PP | PTK2B |
| SRF | PP | ELK1 |
| STAT1 | PP | CSF1 |
| STAT1 | PP | EGF |
| STAT1 | PP | IFNG |
| STAT1 | PP | IL2 |
| STAT1 | PP | YAPK1 |
| STAT3 | PP | HGF |
| STAT3 | PP | YAPK14 |
| STAT3 | PP | STAT5A |
| STAT5A | PP | BCL2L1 |
| STAT5A | PP | IL5 |
| STAT5A | PP | IL5 |
| STAT6 | PP | IL4 |
| STAT6 | PP | IL4R |
| SYK | PP | FegammaRI |
| SYK | PP | LYN |
| T cell transition | PP | JUN |
| factor NFATC | | |
| TAC1 | PP | FOS |
| TAF1 | PP | CCND1 |
| TAP1 | PP | IFNG |
| T-cell receptor | PP | IFNG |
| T3 complex | | |
| T-cell receptor | PP | HAPK1 |
| T3 complex | | |
| TFIID | PP | TP53 |
| TGFA | PP | EGFR |
| TGFA | PP | FNI |
| TGFA | PP | HGF |
| TGFA | PP | [symbol] GF1 |
| TGFA | PP | IL1B |
| TGFA | PP | IL6 |
| TGFA | PP | RAF1 |
| TGFA | PP | TP53 |

TABLE 55

| Protein A | | Protein B |
|---|---|---|
| TGFB1 | PP | ADP |
| TGFB1 | PP | alphaVbeta6 |
| TGFB1 | PP | CRP |
| TGFB1 | PP | EDN1 |
| TGFB1 | PP | HIF1A |
| TGFB1 | PP | IER2 |
| TGFB1 | PP | ILIR1 |
| TGFB1 | PP | IL8 |
| TGFB1 | PP | ITGAE |
| TGFB1 | PP | LCP1 |
| TGFB1 | PP | LTBP1 |
| TGFB1 | PP | N [symbol] F |
| TGFB1 | PP | NYB13 |
| TGFB1 | PP | NYP14 |
| TGFB1 | PP | HYB |
| TGFB1 | PP | HGFB |
| TGFB1 | PP | PDGFA |
| TGFB1 | PP | PDGFB |
| TGFB1 | PP | PTAS3 |
| TGFB1 | PP | PKC |
| TGFB1 | PP | protein-glutamine gamma- |
| | | glutanyltransferase |
| TGFB1 | PP | PTH |
| TGFB1 | PP | RHF7 |
| TGFB1 | PP | SPARC |
| TGFB1 | PP | TFE3 |
| TGFB1 | PP | TGFB3 |
| TGFB1 | PP | TIEG |
| TGFB1 | PP | TIEG2 |
| TGFB1 | PP | vitronectin receptor |
| | | (integrin) |
| TGFB1 | PP | VTN |
| TGFB2 | PP | TGFB1 |
| TGFbetaR | PP | TGFB1 |
| THPO | PP | IL6 |
| thrombin | PP | PLCG1 |
| TLR2 | PP | IL6 |
| TNF | PP | Achronobacter iophagus |
| | | collagenase |
| TNF | PP | AGTR1 |
| TNF | PP | ANGPT2 |
| TNF | PP | B2K |
| TNF | PP | CADPS |
| TNF | PP | calpain |
| TNF | PP | CCLI1 |
| TNF | PP | CD14 |
| TNF | PP | CD69 |
| TNF | PP | CEACAV5 |
| TNF | PP | CRADD |
| TNF | PP | CSF1 |
| TNF | PP | ECE1 |
| TNF | PP | EDN1 |
| TNF | PP | endothelin |
| TNF | PP | EP300 |
| TNF | PP | FADD |
| TNF | PP | FCER2 |
| TNF | PP | FN1 |
| TNF | PP | FS [symbol] 1 |
| TNF | PP | HTATIP |
| TNF | PP | IL1 |
| TNF | PP | IL12B |
| TNF | PP | IL15 |
| TNF | PP | IL18 |
| TNF | PP | IL1A |
| TNF | PP | IL1B |
| TNF | PP | IL1F8 |
| TNF | PP | IL2 |
| TNF | PP | IL2RB |
| TNF | PP | IL7 |
| TNF | PP | IL8 |
| TNF | PP | integrin |
| TNF | PP | interleukin-I receptor |
| | | ligand |
| TNF | PP | ITGB2 |

TABLE 56

| Protein A | | Protein B |
|---|---|---|
| TNF | PP | ITIH4 |
| TNF | PP | JNK |
| TNF | PP | JUND |
| TNF | PP | LBP |
| TNF | PP | LPS |
| TNF | PP | NADCAK1 |
| TNF | PP | NAPK8 |
| TNF | PP | NAPKAPK2 |
| TNF | PP | matrix petalloproteinase |
| TNF | PP | NDDC |
| TNF | PP | NHP14 |
| TNF | PP | NYLK |
| TNF | PP | nadph oxidase |
| TNF | PP | NDUFA1 |
| TNF | PP | NFKB1 |
| TNF | PP | NGF8 |
| TNF | PP | NDS2A |
| TNF | PP | PARP1 |
| TNF | PP | PGF2 alpha |
| TNF | PP | PLA2G10 |
| TNF | PP | PLA2G1B |
| TNF | PP | PLAU |
| TNF | PP | PRKCE |
| TNF | PP | PRKCH |
| TNF | PP | PRKCZ |
| TNF | PP | prostaglandin- |
| | | endoperoxide synthase |
| TNF | PP | protein serine/ |
| | | threonine kinase |
| TNF | PP | protein lyrosine kinase |
| TNF | PP | PTK28 |
| TNF | PP | RAF1 |
| TNF | PP | RAS small monomeric |
| | | GTPase |
| TNF | PP | replication factor C |
| TNF | PP | RIPK1 |
| TNF | PP | RPSGK |
| TNF | PP | T cell transcription |
| | | factor NFATC |
| TNF | PP | TACR1 |
| TNF | PP | TGFA |
| TNF | PP | TNFRSF9 |
| TNF | PP | TNFSF5 |
| TNF | PP | TND1 |
| TNF | PP | TP53 |
| TNF | PP | VLDL |
| TNF | PP | VTN |
| TNFRSF1A | PP | VAPK3 |
| TNFRSF1A | PP | VAPK8 |
| TNFRSF5 | PP | CCL5 |
| TNFRSF5 | PP | IFNG |
| TNFRSF5 | PP | JUN |
| TNFRSF6 | PP | CASP3 |
| TNFRSF6 | PP | CD4 |
| TNFRSF6 | PP | CYCS |
| TNFRSF6 | PP | PARP1 |
| TNFSF10 | PP | TP53 |
| TNFSF5 | PP | CD40 receptor |
| TNFSF5 | PP | IFNG |
| TNFSF6 | PP | CD4 |
| TNFSF6 | PP | CYCS |
| TNFSF6 | PP | JUN |

TABLE 57

| Protein A | | Protein B |
|---|---|---|
| TP53 | PP | ABC81 |
| TP53 | PP | AGT |
| TP53 | PP | AXTN1 |
| TP53 | PP | CASP3 |
| TP53 | PP | CAV1 |
| TP53 | PP | CCNG1 |
| TP53 | PP | CDKN1A |
| TP53 | PP | EGFR |
| TP53 | PP | FNL2 |
| TP53 | PP | GADD45A |
| TP53 | PP | HES1 |
| TP53 | PP | HGF |
| TP53 | PP | HRAS |
| TP53 | PP | NAPK8 |
| TP53 | PP | NSH2 |
| TP53 | PP | progesterone |
| TP53 | PP | PTEN |
| TP53 | PP | RACI |
| TP53 | PP | SLC12A9 |
| TP53 | PP | SUH01 |
| TP53 | PP | TNFSF6 |
| TP53 | PP | TP53I3 |
| TP53 | PP | NIG1 |
| TP73 | PP | NYC |
| TPA | PP | IL6 |
| TPSB1 | PP | TGFB1 |
| TRAF2 | PP | VAPK8 |
| TRAF5 | PP | VAPK8 |
| tumor necrosis | PP | CDKN1A |
| factor | | |
| tumor necrosis | PP | NAPK3 |
| factor | | |
| tumor necrosis | PP | NAPK8 |
| factor | | |
| tumor necrosis | PP | RHOA |
| factor | | |
| TYK2 | PP | IFNG |
| Type I protein gerenyl- | PP | INS |
| geranyltransferase | | |
| USF1 | PP | USF2 |
| VAV1 | PP | NAOK8 |
| VCAN1 | PP | ICAN1 |
| VCAN1 | PP | NAPK14 |
| VEGF | PP | ANGPT2 |
| VEGF | PP | cANP-dependent protein |
| | | kinase catalyst |
| VEGF | PP | CSF1 |
| VEGF | PP | EGFR |
| VEGF | PP | EGR1 |
| VEGF | PP | F3 |
| VEGF | PP | MAPK1 |
| VEGF | PP | PRKCA |
| VEGF | PP | PTGS2 |
| VEGF | PP | RAF1 |
| VEGF | PP | RHOA |
| VEGF | PP | RPSBKP1 |
| VEGF | PP | SERPINE1 |
| VEGF | PP | SHC1 |
| VEGF | PP | SRC |
| VEGF | PP | STAT3 |
| VIP | PP | ADCY2 |
| VIP | PP | CREB1 |
| VIP | PP | LIF |
| WARS | PP | IFNG |

The protein information database 3 also stores information about how the intermolecular interaction between two molecules affects the cellular function.

How the intermolecular interaction between two molecules affects the cellular function is inferred in the following way.

For a molecule set of NN-type, the following steps are taken to infer how the intermolecular interaction between two molecules affects the cellular function. The first step is to select the cellular function to be affected simultaneously by two proteins of the molecule set of interest. It is assumed that when the cellular function X is promoted by protein A and suppressed by protein B, and protein A is on and protein B is off, the molecule set of protein A and protein B promotionally acts on the cellular function X. Conversely, it is assumed that when the cellular function X is suppressed by protein A and promoted by protein B, and protein A is off and protein B is on, the molecule set of protein A and protein B promotionally acts on the cellular function X.

The protein information database 3 stores information about how the intermolecular interaction (between two molecules) affects the cellular function with respect to each protein of the molecule set of NN-type. As its example, FIG. 3 shows the relation between the molecule set of NN-type relating to TP53 molecule and the cellular function that is promoted in response to on/off of the two proteins.

In the case of molecule set of NN-type involving TP53 and ABCC1 shown in FIG. 3A, it promotes no cellular function when TP53 is on and ABCC1 is off, and it promotes synthesis and motility as the cellular function when TP53 is off and ABCC1 is on.

In the case of molecule set of NN-type involving TP53 and telomerase shown in FIG. 3B, it promotes death and aging as the cellular function when TP53 is on and telomerase is off, and it promotes proliferation as the cellular function when TP53 is off and telomerase is on.

In the case of molecule set of NN-type involving TP53 and FGF2 shown in FIG. 3C, it promotes apoptosis, metabolic catabolism, aging, DNA fragmentation, death, and depolarization as the cellular function when TP53 is on and FGF2 is off, and it promotes mitosis, chromosome DNA replication, replication, amplification, synthesis, growth rate increase, advance of cell cycle to S phase, motility, angiogenesis, proliferation, and advance of cell state to G1 phase as the cellular function when P53 is off and FGF2 is on.

In the case of molecule set of NN-type involving TP53 and TERT shown in FIG. 3D, it promotes apoptosis, death, DNA damage recognition, and aging as the cellular function when TP53 is on and TERT is off, and it has no effect on the cellular function when P53 is off and TERT is on.

In the case of molecule set of NN-type involving TP53 and HSPA4 shown in FIG. 3E, it promotes apoptosis, DNA fragmentation, and death as the cellular function when TP53 is on and HSPA4 is off, and it has no effect on the cellular function when P53 is off and HSPA4 is on.

In the case of molecule set of NN-type involving TP53 and TXNRD1 shown in FIG. 3F, it has no effect on the cellular function when TP53 is on and TXNRD1 is off, and it promotes cell proliferation when TP53 is off and TXNRD1 is on.

Thus, the protein information database 3 stores any molecule set of NN-type involving other proteins than PT53 to show what cellular function is promoted when which protein is on.

For the relation between the cellular function and interaction between two molecules in the molecule set of PN-type, the molecule set of PN-type is assumed to positively act (POS) on the cellular function Y which is promoted by protein A and suppressed by protein B when promotive action is brought from protein A to protein B. Likewise, it is assumed to negatively act (NEG) on the cellular function Z which is suppressed by protein A and promoted by protein B.

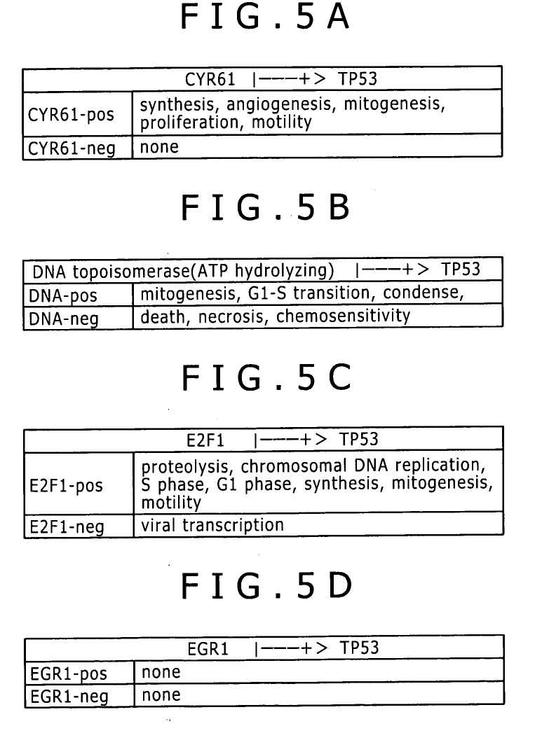

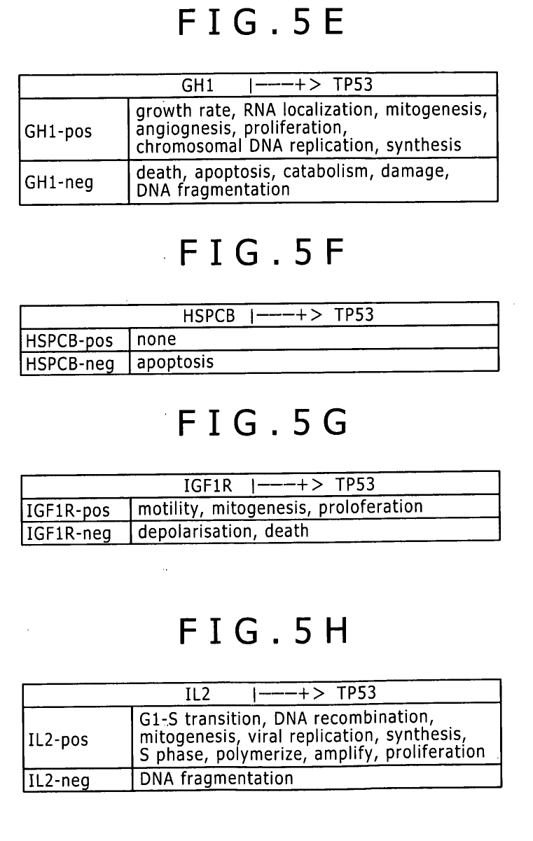

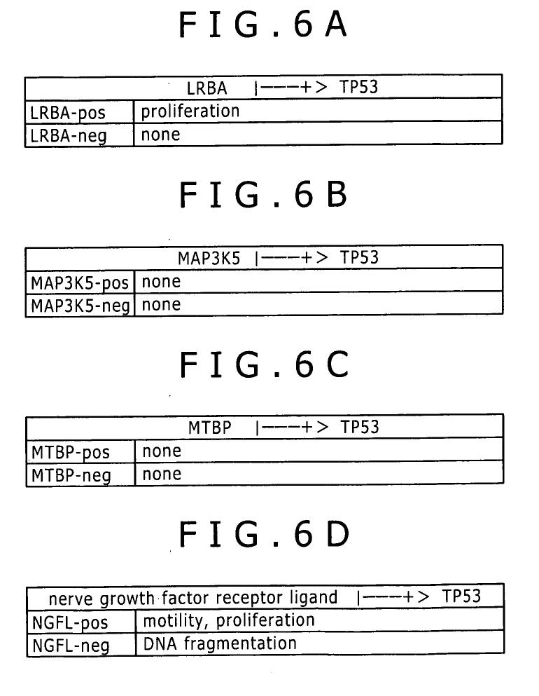

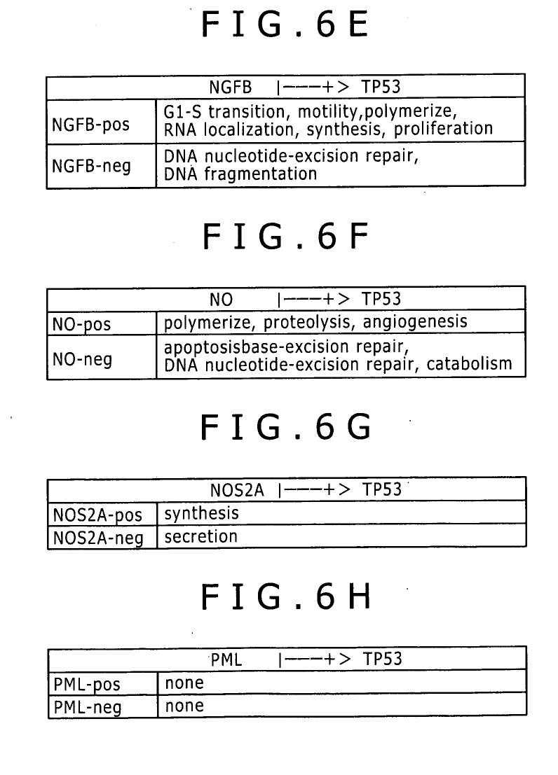

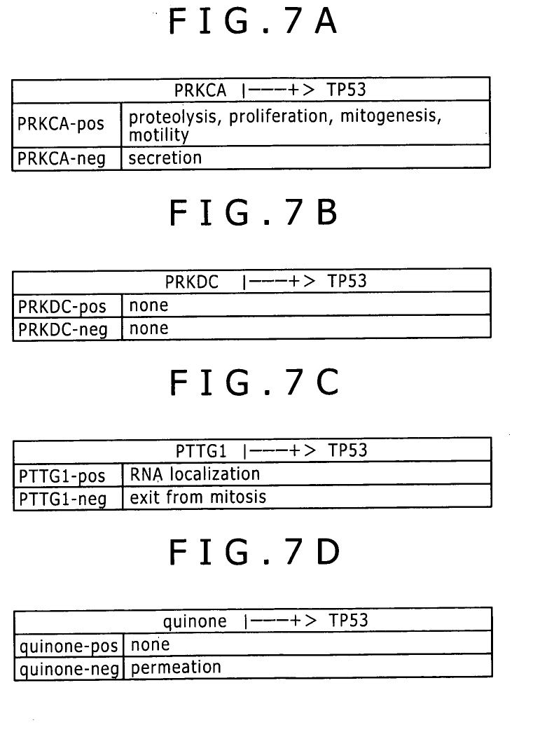

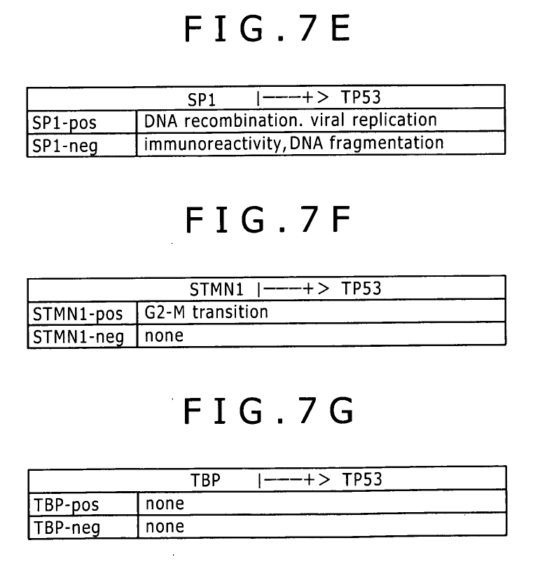

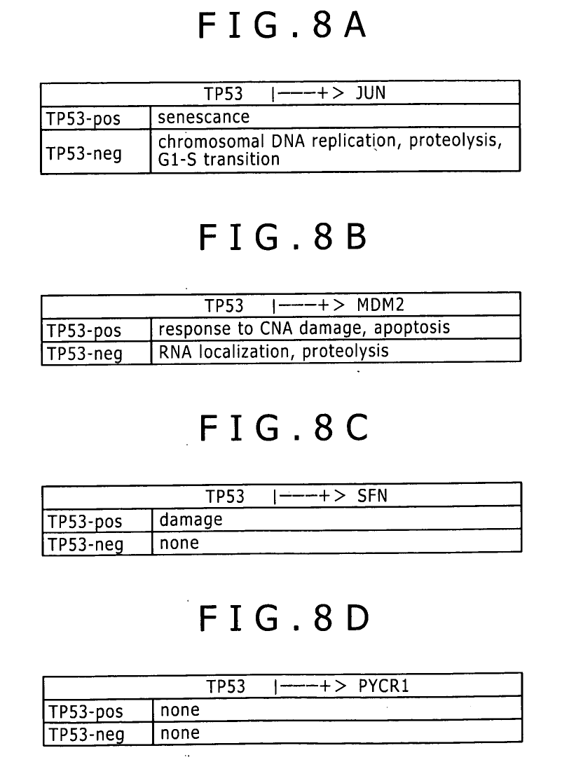

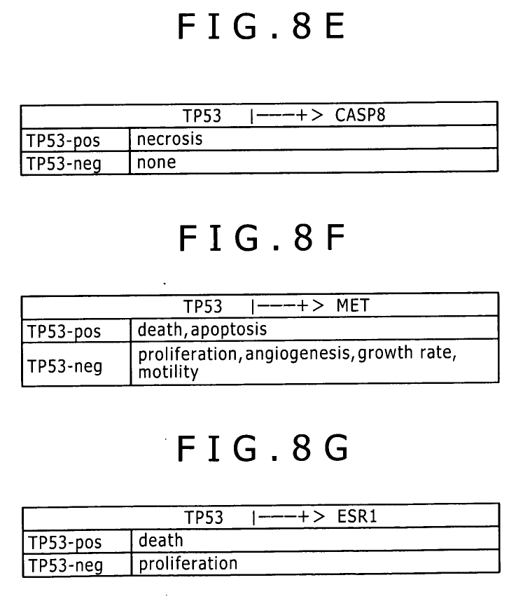

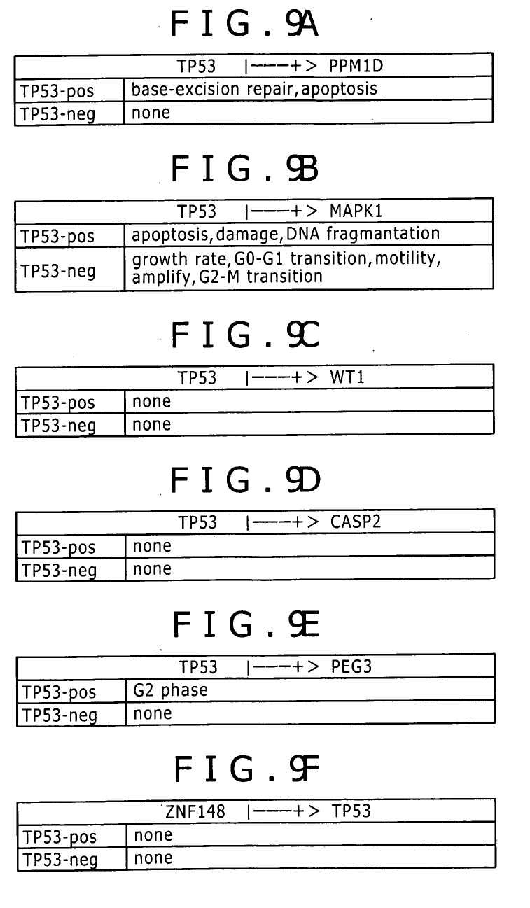

The protein information database 3 stores information about the relation between the cellular function and the interaction between two molecules for proteins involved in the molecule set of PN-type. This is illustrated in FIGS. 4 to 9, which show the relation between the cellular function and the molecule set of PN-type involving TP53.

In the case of molecule set of PN-type involving ADP and TP53 as shown in FIG. 4A, ADP promotes the function of synthesis, motility, mitogenesis, and polymerization, and ADP suppresses the function of damage and DNA damage recognition. In the case of molecule set of PN-type involving AGTR2 and TP53 as shown in FIG. 4B, AGTR2 promotes the function of synthesis and motility, and AGTR2 suppresses the function of cell permeability.

In the case of molecule set of PN-type involving catenin and TP53 as shown in FIG. 4C, catenin promotes the function of motility and proliferation, and catenin does not suppress any specific function. In the case of molecule set of PN-type involving CCNE1 and TP53 as shown in FIG. 4D, CCNE1 promotes the function of chromosomal DNA replication, condensation, proteolysis, synthesis, G1-S transition, instability, advance of cell cycle to S phase, motility, mitogenesis, and advance of cell cycle to G1 phase, and CCNE1 does not suppress any specific function.

In the same way as mentioned above, proteins involved in the molecule sets of PN-type shown in FIGS. 4E to 4H and FIGS. 5 to 9 given later promote and suppress the cellular functions. This information is stored in the protein information database 3.

The relation between the cellular function and interaction between two molecules is inferred in the same way as mentioned above for the molecule set of PP-type, that is, two proteins are regarded as “POS” (for promotion) and “NEG” (for suppression), respectively, if they promote and suppress the specifically selected cellular function on which they act simultaneously.

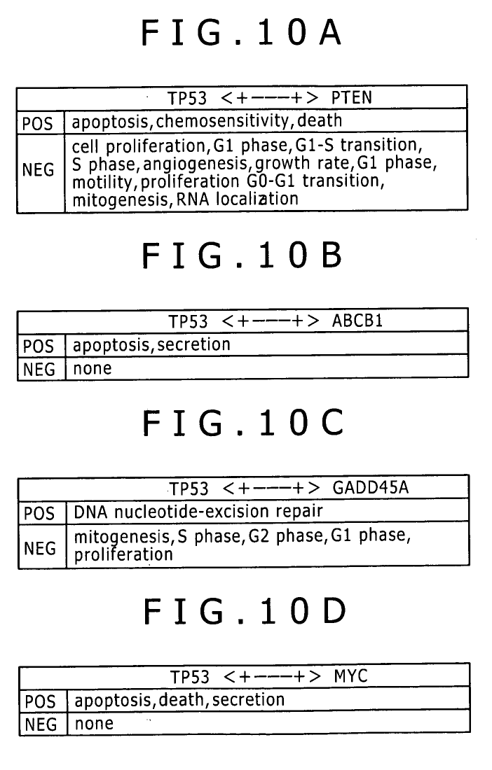

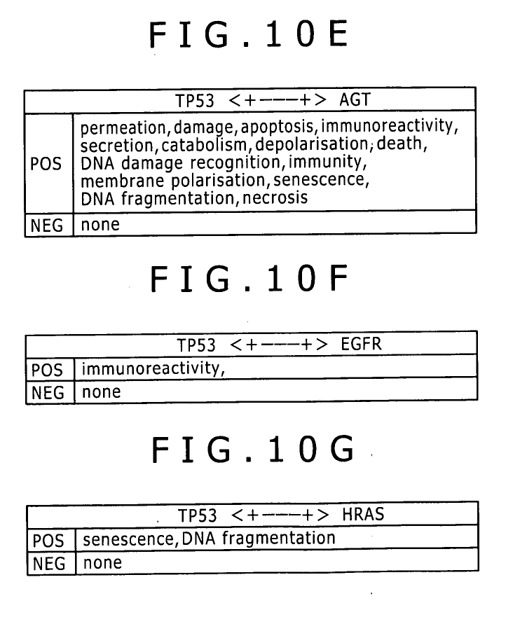

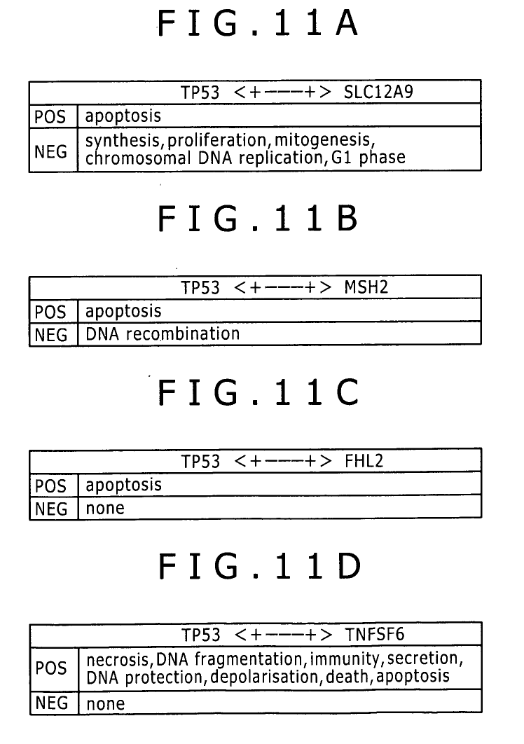

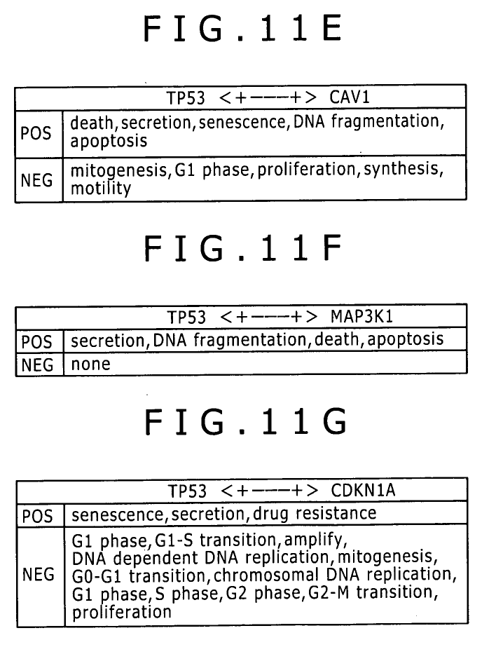

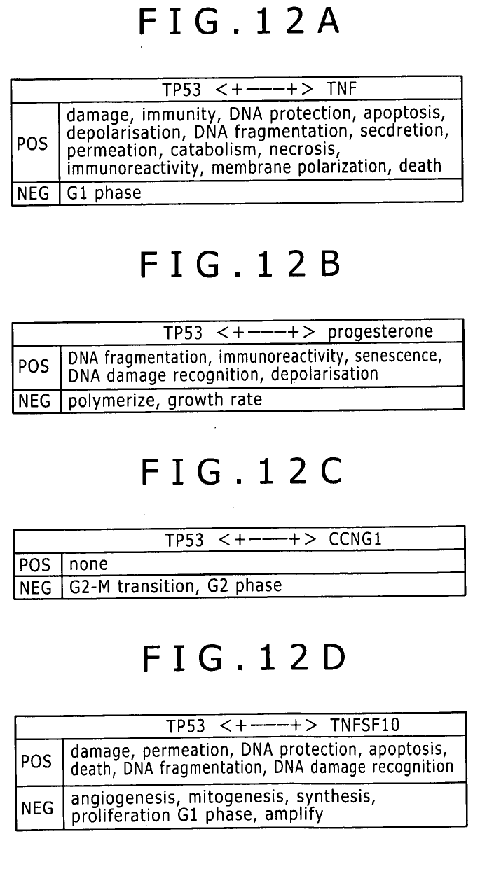

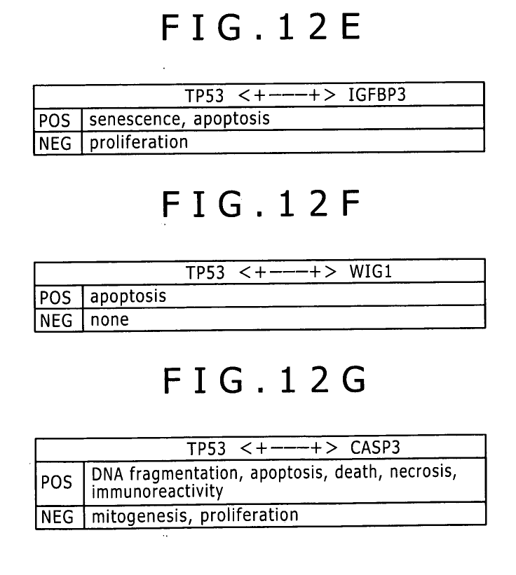

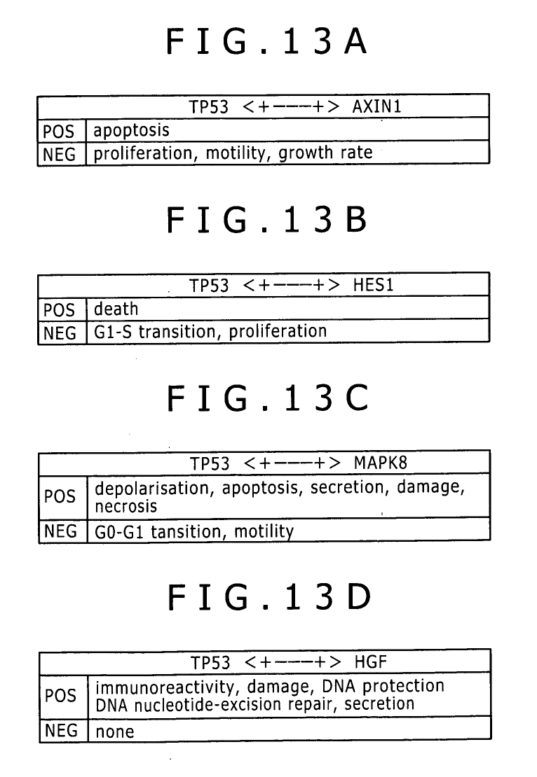

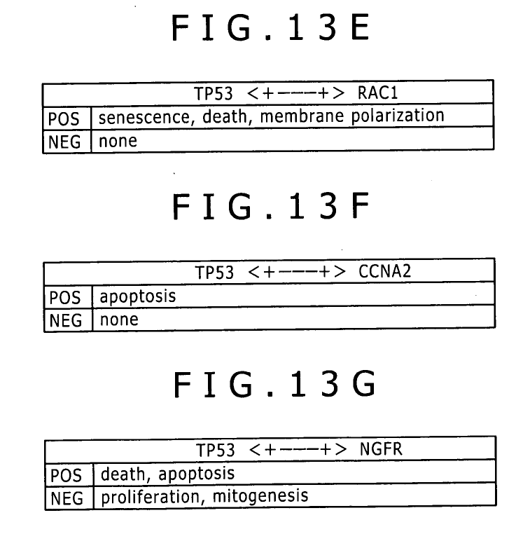

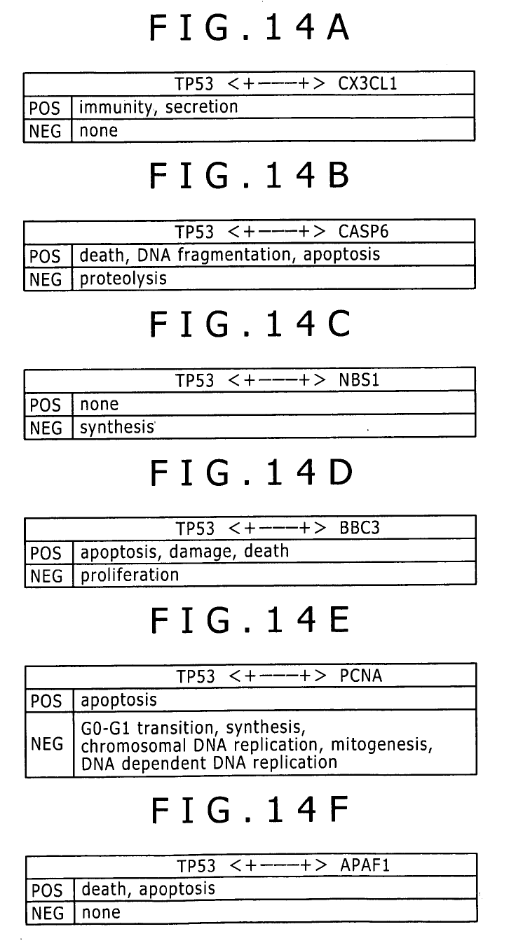

The protein information database 3 stores information about relation between the interaction of two molecules and the cellular function for the molecule set of PP-type involving various proteins. FIGS. 10 to 14 show, as some of their examples, how the cellular function is affected by the molecule set of PN-type involving TP53 molecule.

The molecule set of PN-type involving TP53 and PTEN as shown in FIG. 10A promotes the cellular function of apoptosis, chemosensitivity, and death if both proteins act for promotion, and it promotes the function of cell proliferation, advance of cell cycle to G1 phase, G1-S transition, advance of cell cycle to S phase, angiogenesis, growth rate increase, G0-G1 transition, mitogensis, and RNA localization if both proteins act for suppression. The molecule set of PN-type involving TP53 and ABCB1 as shown in FIG. 10B promotes the cellular function of apoptosis and secretion (release from cells of molecules produced inside cells) if both proteins act for promotion, and it does not promote any specific cellular function if both proteins act for suppression.

The molecule set of PN-type involving TP53 and GADD45A as shown in FIG. 10C promotes the cellular function of DNA nucleotide-excision repair if both proteins act for promotion, and it promotes the function of mitogenesis, S phase, G2 phase, G1 phase, and proliferation if both proteins act for suppression. The molecule set of PN-type involving TP53 and MYC as shown in FIG. 10D promotes the cellular function of apoptosis, death, and secretion, if both proteins act for promotion, and it does not promote any specific cellular function if both proteins act for suppression.

In the same way as mentioned above, proteins involved in the molecule sets of PN-type shown in FIGS. 10E to 10G and FIGS. 11 to 14 given later promote and suppress the cellular functions. This information is stored in the protein information database 3.

Now, the description of FIG. 1 is revisited.

The protein information analyzing unit 4 includes the protein expression ratio arithmetic unit 21, the point accumulating unit 22, the factor setting unit 23, the operating input acquisition unit 24, the database building and processing unit 25, the network building unit 26, the target molecule inferring unit 27, and the result output unit 28.

The protein expression ratio arithmetic unit 21 receives from the mRNA expression analyzing unit 2 (or the protein kit 7) information about the amount of target protein expressed in normal cells and information about the amount of target protein expressed in sample cells. It compares the amount of target protein expressed in normal cells with the amount of target protein expressed in sample cells and calculates the increase or decrease of the amount of target protein expressed. It supplies the thus obtained value as the protein index to the point accumulating unit 22.

The point accumulating unit 22 receives the value of protein index from the protein expression ratio arithmetic unit 21 and calculates the accumulated value of scores for individual cellular functions by using the value of protein index of two proteins constituting the molecule set of NN-type, PN-type, and PP-type stored in the protein information database 3 and the value of the factor set up in the factor setting unit 23. If the point accumulating unit 22 gives a positive value of score for the cellular function, it means that the cell for detection promotes the cellular function; otherwise, it means that the cell for detection suppresses the cellular function.

The point accumulating unit 22 performs arithmetic process in the following manner for the molecule sets of NN-type, PN-type, and PP-type.

Association with cellular function and scoring are carried out as follows for the molecule set of NN-type involving INS and IFNG.

The point accumulating unit 22 receives from the protein expression ratio arithmetic unit 21 the values of protein index for the two proteins constituting the molecule set of NN-type and then calculates the absolute value of the difference between the two values. Subsequently, it assigns the absolute value to be positive or negative according to whether each cellular function is promoted or suppressed, and multiplies it by the factor set up by the factor setting unit 23, thereby giving the score of the cellular function associated with the molecule set.

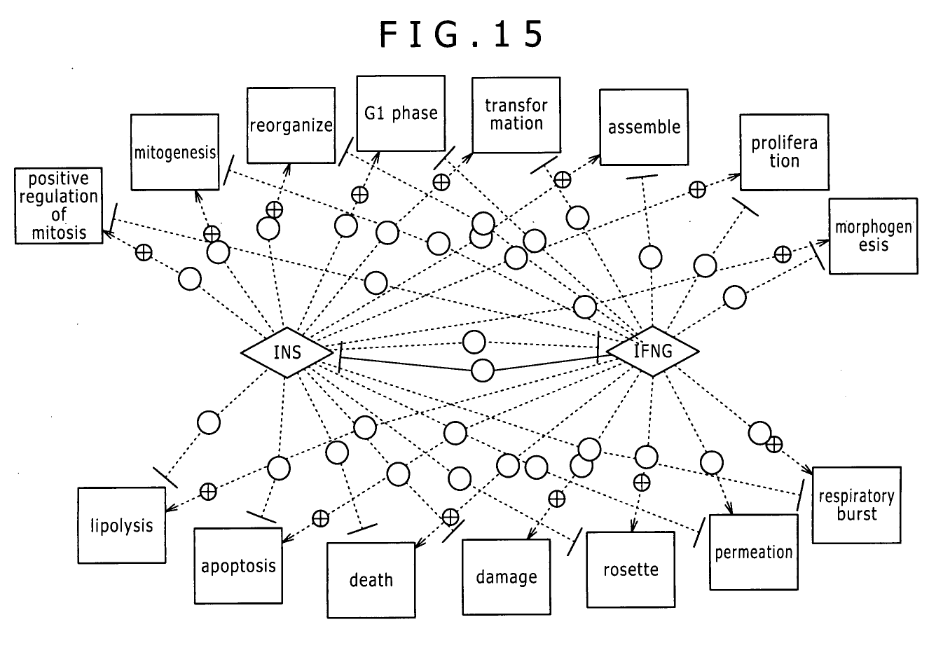

The cellular function associated with INS and IFNG for the molecule set of NN-type is classified into two categories as shown in FIG. 15. The first category includes those cellular functions which are promoted by INS (denoted by--->) and suppressed by IFNG (denoted by---|). The second category includes those cellular functions which are suppressed by INS and promoted by IFNG. Those cellular functions which are promoted by INS and suppressed by IFNG include positive regulation of mitosis, mitogenesis, reorganization, G1 phase, transformation, assembling, proliferation, and morphogenesis. Those cellular functions which are suppressed by INS and promoted by IFNG include lypolysis, apoptosis, death, damage, rosette, permeation, and respiratory burst.

The point accumulating unit 22 receives the protein index of INS and the protein index of IFNG from the protein expression ratio arithmetic unit 21. If the difference between the two indexes for the cellular function promoted by INS is larger than 0, it adds a positive sign to the absolute value of the difference. If the difference between the two indexes is smaller than 0, then it adds a negative sign to the absolute value of the difference. It multiplies the signed value by the factor set up in the factor setting unit 23. The resulting value is the score of the cellular function controlled by the two proteins (shown in FIG. 15).

Association with cellular function and scoring are carried out as follows for the molecule set of PN-type involving INS and JUN.

The point accumulating unit 22 receives from the protein expression ratio arithmetic unit 21 the value of protein index for either of the two proteins constituting the molecule set of PN-type which is promoted. It makes the value positive or negative according to whether the cellular function is promoted or suppressed and then multiplies it by the factor set up by the factor setting unit 23, thereby giving the score of the cellular function associated with the molecule set.

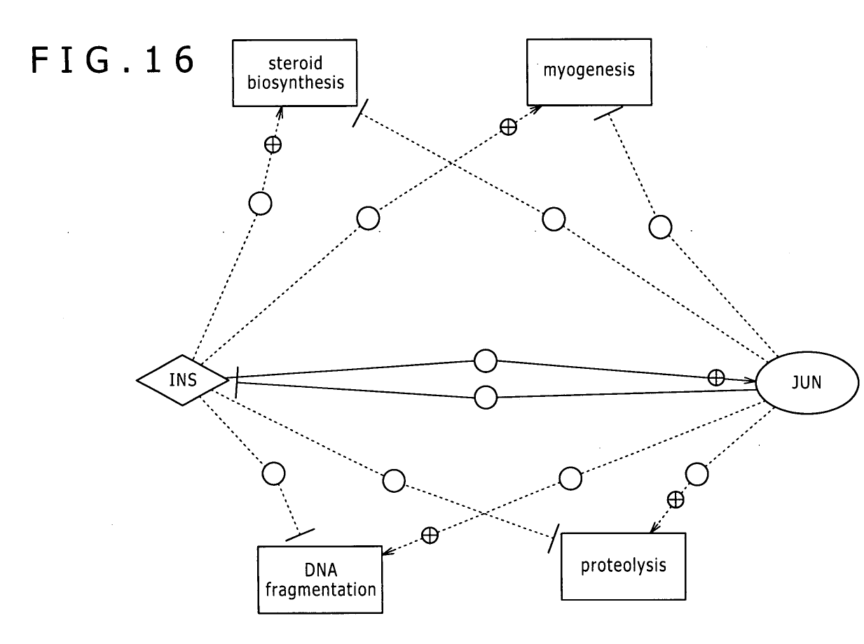

The cellular function associated with INS and JUN for the molecule set of PN-type is classified into two categories as shown in FIG. 16. The first category includes those cellular functions which are promoted by INS and suppressed by JUN. The second category includes those cellular functions which are suppressed by INS and promoted by JUN. Those cellular functions which are promoted by INS and suppressed by JUN include steroid biosynthesis and mitogenesis. Those cellular functions which are suppressed by INS and promoted by JUN include DNA fragmentation and proteolysis. The point accumulating unit 22 makes positive the value of the protein index of INS for the cellular function promoted by the promoting protein (INS in the case in FIG. 3), and it makes negative the value of the protein index for the cellular function suppressed by INS. Then it multiplies the resulting value by the factor set up by the factor setting unit 23, thereby giving the score of the cellular function (shown in FIG. 16) controlled by the two proteins.

Association with cellular function and scoring are carried out as follows for the molecule set of PP-type involving TNF and TP53.

The point accumulating unit 22 receives from the protein expression ratio arithmetic unit 21 the values of protein index for the two proteins constituting the molecule set of PP-type and then calculates their product. Subsequently, it assigns the product to be positive or negative according to whether each cellular function is promoted or suppressed, and multiplies it by the factor set up by the factor setting unit 23, thereby giving the score of the cellular function associated with the molecule set.

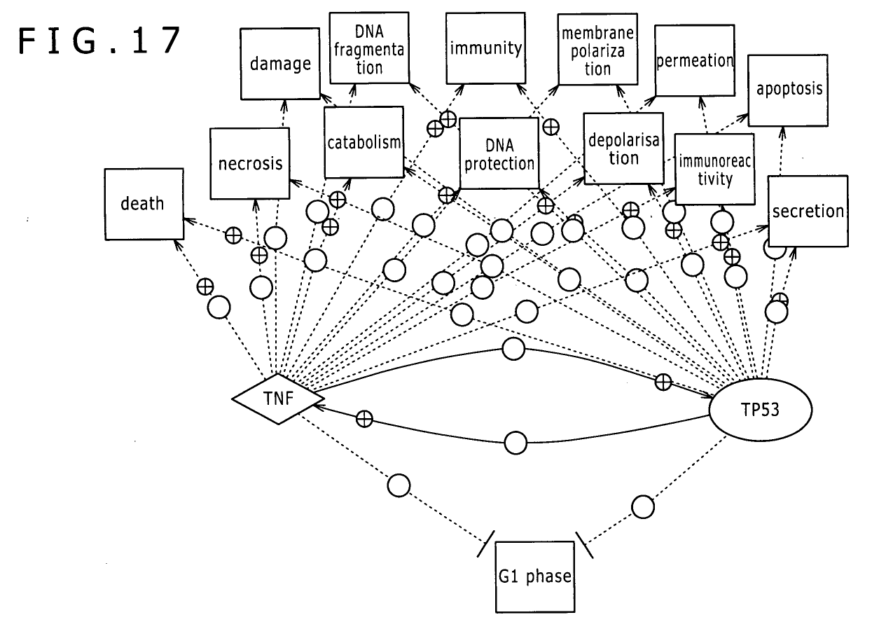

The cellular function associated with INS and TP53 for the molecule set of PP-type is classified into two categories as shown in FIG. 17. The first category includes those cellular functions which are promoted by both proteins. The second category includes those cellular functions which are suppressed by both proteins. Those cellular functions which are promoted by both proteins include death, necrosis, damage, apoptosis, and secretion. Those cellular functions which are suppressed by both proteins include advance of cell cycle to G1 phase. The point accumulating unit 22 multiplies the product of indexes of both proteins by the factor set up by the factor setting unit 23 in the case of the cellular functions promoted by both proteins involved in the molecule set, thereby giving the score of the cellular function associated with the molecule set. The point accumulating unit 22 also multiplies the negative value of the product of indexes of both proteins by the factor set up by the factor setting unit 23 in the case of the cellular functions suppressed by both proteins involved in the molecule set, thereby giving the score of the cellular function associated with the molecule set.

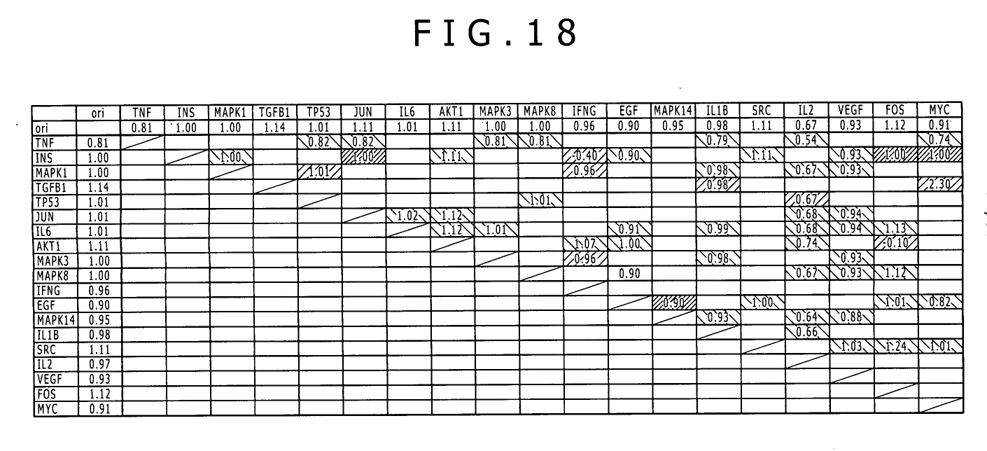

The protein index is calculated in the following manner which is explained with reference to FIG. 18 for typical 19 kinds of proteins involving cell interactions on the basis of data showing the expression of mRNA of DAOY (cultured cell of human medulloblastoma).

In FIG. 18, “ori” (original) denotes the ratio of the occurrence of a specific protein (out of 19 proteins) in the control target to the occurrence of a specific protein (out of 19 proteins) in the sample target.

The combination of 19 proteins (shown in FIG. 18) gives the result of calculations for the molecule sets of NN-type. If the protein corresponding to the ordinate has a larger index than that corresponding to the abscissa, it means that the former is in the promotion (POS) side and hence the difference (in terms of absolute value) between the two indexes is obtained. If the protein corresponding to the ordinate has a smaller index than that corresponding to the abscissa, it means that the former is in the suppression (NEG) side and hence the difference (in terms of absolute value) between the two indexes is obtained. In other words, the difference between indexes of INS and IFNG is −0.40 for the corresponding cellular functions explained with reference to FIG. 15. This value is multiplied by the factor and the resulting product is assigned to be positive or negative according to whether the protein is in the promotion side or suppression side. The same calculations as above are performed on other molecule sets of NN-type.

The combination of 19 proteins (shown in FIG. 18) indicates how the protein index of the protein in the promotion side is associated with the cellular function for the molecule set of PN-type. In other words, it gives the score (calculated by multiplying the cellular function promoted by INS by the factor of 1.00) for the molecule set of INS and JUN (explained above with reference to FIG. 16). The same calculations as above are performed on other molecule sets of PN-type.

The combination of 19 proteins (shown in FIG. 18) indicates the product of the index of the protein corresponding to the ordinate and the index of the protein corresponding to the abscissa. The molecule set involving TNF and TP53 (which has been explained with reference to FIG. 17) gives the score obtained by multiplying −0.82 by the factor for the cellular function promoted by both proteins. The same calculations as above are performed on other molecule set of PP-type.

As mentioned above, the point accumulating unit 22 calculates the score of the cellular function (as explained with reference to FIGS. 15 to 18) for the molecule sets of NN-type, PN-type, and PP-type, and then accumulates the scores of individual cellular functions and supplies the results to the result output unit 28 and the target molecule inferring unit 27.

The factor setting unit 23 sets up the factor for score accumulation to be executed by the point accumulating unit 22. The factor should preferably be set up such that it takes the largest value for NN-type and the smallest value for PP-type. If there is a molecular bond between two molecules involved in the molecule set, the factor should be multiplied by a prescribed value larger than 1. These factors are previously obtained by experiment and experience; they may be set up in the factor setting unit 23 or may be changed by the user through processing in the operation input acquisition unit 24.

The operation input acquisition unit 24 is an input device such as keyboard, mouse, touch pad, and touch panel, which receives inputs in response to the user's operation. It permits the user to change the setting of the factor in the factor setting unit 23, to change the value of protein index in the simulation by the target molecule inferring unit 27 (mentioned later), and to update the database in the database building and processing unit 25. It supplies the entry to the factor setting unit 23, the target molecule inferring unit 27, and the database building and processing unit 25.

The database building and processing unit 25 updates and supplements various kinds of information stored in the protein information database 3 according to the user's input (which is supplied from the operating input acquisition unit 24) or database externally supplied through the network interface (not shown).

The target molecule inferring unit 27 performs simulation to infer the target molecule on the basis of score for each cellular function obtained from processing by the point accumulating unit 22.

The target molecule inferring unit 27 simulates the change of score for cellular function which occurs when the protein index of specific molecule changes in the expression of mRNA of DAOY (cultured cell of human medulloblastoma), which was explained above with reference to FIG. 18.

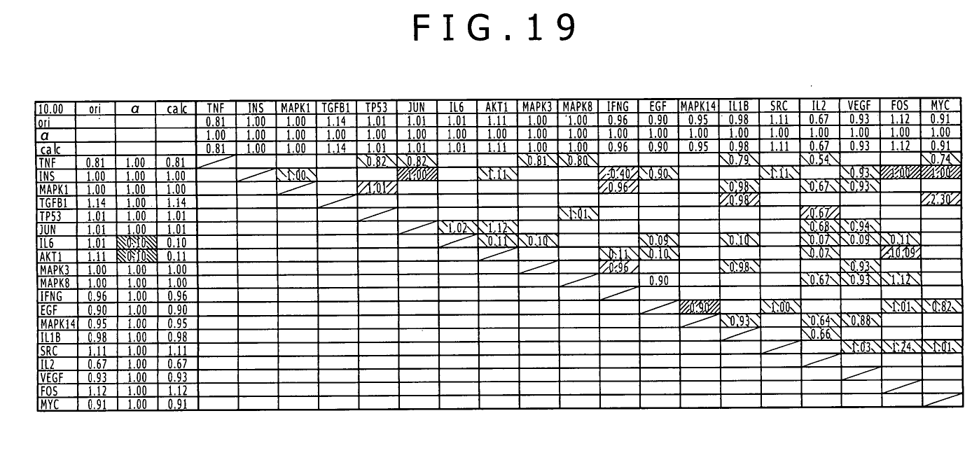

FIG. 19 shows the value (which is not yet multiplied by the factor) as the base of the score for the cellular function corresponding to individual molecule sets. The factor α for the protein index of AKT1 and IL6 is 0.1. Incidentally, AKT1 and IL6 are included in the 19 proteins (involving the expression of mRNA of DAOY) which were explained above with reference to FIG. 18.

The value in FIG. 19 differs from that in FIG. 18 in that the score for cellular function associated with FOS and AKT1 for the molecular set of NN-type is one which is obtained by multiplying −10.09 by a prescribed factor, because each protein index of AKT1 and IL6 is multiplied by 0.1. In addition, there is a difference between FIG. 19 and FIG. 18 in the value as the base for the score of cellular function associated with the molecule set of PP-type involving AKT1 and IL6.

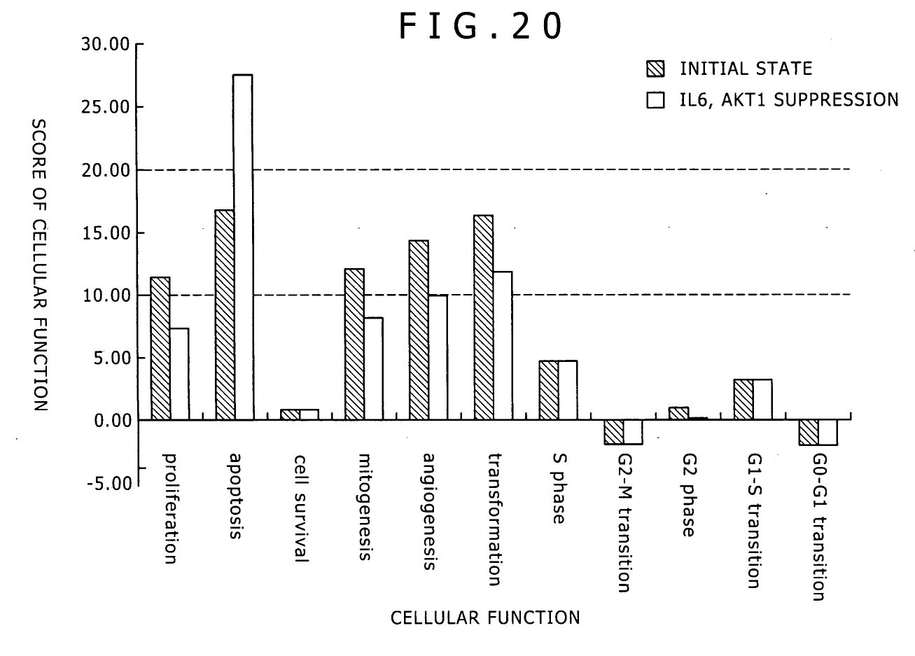

FIG. 20 shows how the score for cellular function changes when the base value of score for cellular function is multiplied by the factor and the results are accumulated as shown in FIGS. 18 and 19. The cellular functions which change in accumulated values as shown in FIG. 20 are those which are seriously associated with cell proliferation and cell death. They include proliferation, apoptosis, cell survival, mitogenesis, angiogenesis, transformation, S phase, G2 phase, G2-M transition, G2 phase, G1-S transition, and G0-G1 transition.

As shown in FIG. 20, the score for cellular functions relating to proliferation, mitogenesis, angiogenesis, and transformation decreases and the score for cellular functions relating to apoptosis increases, with the factor α for AKT1 and IL6 set at 0.1.

The foregoing suggests that any treatment (with an anticancer agent, for example) to suppress the function of proteins (AKT1 and IL6) causes at least the cultured cell of human medulloblastoma (DAOY) to dye rather than proliferate. Finding a combination of proteins for the most remarkable effect will help search for the candidate of target molecule as an anticancer agent.

The target molecule inferring unit 27 may also be designed such that it performs simulation to infer the target molecule based on the protein network model built up by the network building unit 26.

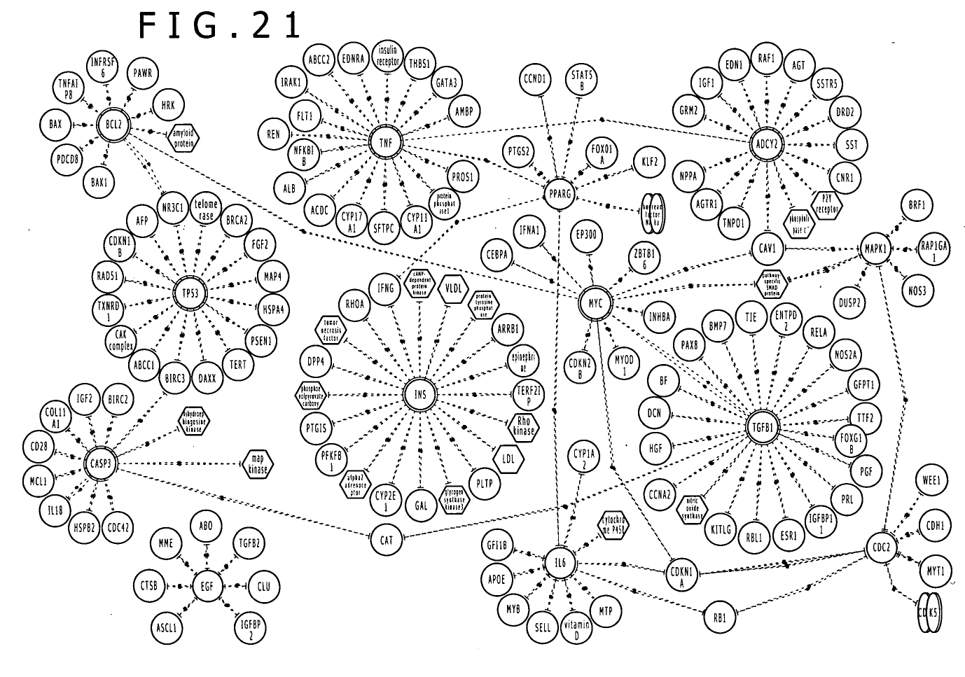

The network building unit 26 builds up the molecule network based on the information stored in the protein information database 3. FIG. 21 shows the molecule network drawn from the molecule set of NN-type. The molecule network may also be drawn for the molecule sets of other types. The molecule network is drawn separately for individual categories, and it is also drawn three-dimensionally according to relations among individual proteins.

Any increase or decrease of the index of a certain protein in the protein network model built up by the network building unit 26 affects the index of other proteins connected with the network. The target molecule inferring unit 27 simulates the change of index of individual proteins in the network to infer how an increase or decrease of protein index at one node affects the protein index at other nodes (adjacent to the node in which the protein index has changed), on the assumption that the effect in the first adjacent node is 50%, the effect in the second adjacent node is 30%, the effect in the third adjacent node is 10%, and so on. The protein network model built up by the network building unit 26 consists of more than one molecule network (similar to that shown in FIG. 21) associated with one another, with their nodes connected with one another in a very complicated manner. It will permit more accurate inference of target molecules if it is modified such that consideration is given to the presence of the nodes which are affected through more than one route as the protein index increases or decreases.

The target molecule inferring unit 27 repeats the process of accumulating the score of cellular function by using the result of the simulation which has been carried out by means of the molecule network, thereby inferring the target molecule.

The result output unit 28 receives an accumulated score of cellular function from the point accumulating unit 22 or receives the result of inference of the target molecule from the target molecule inferring unit 27, and then delivers it to either or both of the result display unit 5 and the result analyzing unit 6.

The result display unit 5 consists of a display device such as CRT and LCD. It displays the result of accumulated score of cellular function or the result of inference of the target molecule which has been received from the result output unit 28. The user will be able to perform input operation to infer the target molecule by reference to the result of accumulated score for cellular function which is displayed on the result display unit 5.

The result analyzing unit 6 accumulates the result of accumulated score for cellular functions or the result of inference of the target molecule (which has been received from the result output unit 28) and then performs analysis according to need.

To be concrete, the result analyzing unit 6 accumulates chronologically the result of accumulated score for cellular functions of the same test subject and analyses the chronological change, so that it permits one to correctly judge whether or not the target protein has decreased as the result of medication to the test subject during the specific period. Moreover, it also permits one to confirm the effect (increase or decrease in expression) on other proteins or the effect on other cellular function by medication in that period.

This description is based on the assumption that the result analyzing unit 6 is independent of the protein information analyzing unit 4. However, the former may be included in the latter.

The protein analyzing system according to the present invention permits one to analyze in a simple manner any system anomaly of disease caused by anomalous molecule network in cells (such as cancer).

In other words, the protein analyzing system according to the present invention classifies interactions between two molecules into five categories which are NN-type for two proteins suppressing each other, PN-type for two proteins, with the first one promoting the second one and the second one suppressing the first one, PP-type for two proteins promoting each other, P-type for two proteins, with the first one only promoting the second one, and N-type for two proteins, with the first one only suppressing the second one, and calculates and accumulates the score for the cellular function associated with the pair of proteins falling under any of these categories, thereby digitizing the cellular function. Combining this result with the variation of cellular function makes it possible to infer the system structure of cells.

The present invention makes it possible to analyze the relation between the cellular function and the intermolecular action of proteins instead of merely paying attention to a single molecule. Therefore, it permits one to investigate the change in cellular function which occurs when the amount of specific proteins expressed fluctuates. This capability may be used to simulate a combination to restore the normal state by changing the anomalous cellular function (resulting from cancer, for example). In this way it is possible to infer the target molecule important for medical treatment.

The target molecule important for medical treatment will be inferred by means of the molecule network consisting of nodes representing proteins and links representing interactions classified into five categories mentioned above. In this way it is possible to infer the target molecule more accurately.

Once a correct target molecule is inferred, it would be very useful to establish an adequate way of medication to restore the anomalous system resulting from diseases.

In what follows, the process for analysis by means of the protein analyzing system will be described with reference to FIG. 22 (flow chart).

In Step S1, the chip forming unit 1 prepares a DNA chip to determine the amount of protein expressed (for analysis).

One DNA chip has more than one probe, so that it can determine the amount of more than one protein expressed.

In Step S2, the mRNA expression analyzing unit 2 carries out hybridization for the normal cell and the sample cell. To be concrete, this step is carried out as follows. The DNA chip, which has been prepared in Step S1, is given dropwise a target for control and a target for detection. The target for control is produced by using complementary DNA (cDNA) which has been replicated by reverse transcription from mRNA collected from normal cells. The target for detection is produced by reverse replication of complementary DNA (cDNA) from mRNA collected from sample cells. The probe and target are bound together (hybridized) through the reaction to form the complementary strands (double-strands) between the nucleic acids having the complementary base sequence.

In Step S3, the mRNA expression analyzing unit 2 calculates the amount of target protein expressed in normal cells and the amount of target protein expressed in sample cells, and then it sends the result to the protein expression ratio arithmetic unit 21 of the protein information analyzing unit 4.

The detailed procedure for Step S3 includes cleaning of the DNA chip, on which hybridization has occurred, and addition of an intercalator which emits fluorescence upon irradiation with exciting light, then the intercalator binds with the probe which has been hybridized. The intercalator binds with the probe in such a way that it does not enter between the probe and the target if they are not hybridized and it enters between the probe and the target only if they are hybridized. Upon irradiation with exiting light, the intercalator emits fluorescence, which is subsequently condensed by an object lens or the like and separated from exciting light by a prism. The condensed and separated fluorescence enters a photodiode for image analysis and calculation of the amount of target protein expressed.

In Step S4, the protein expression ratio arithmetic unit 21 of the protein information analyzing unit 4 calculates an increase or decrease in the amount of target protein expressed in the sample cells in comparison with the amount of target protein in the normal cells. Subsequently, it sends the result of calculations to the point accumulating unit 22. In other words, the protein expression ratio arithmetic unit 21 calculates the protein index on the basis of the amount of target protein (control) expressed in the normal cells and the amount of target protein expressed in the sample cells, both of which have been supplied from the mRNA expression analyzing unit 2. Then, it sends the result to the point accumulating unit 22.

In Step S5, the point accumulating unit 22 calculates the point accumulation (mentioned later) according to the flow sheet shown in FIG. 23. It obtains the accumulated value of the score times the factor for each cellular function, and it sends the result to the result output unit 28 and the target molecule inferring unit 27.

In Step S6, the result output unit 28 sends the result obtained in Step S5 to either or both of the result display unit 5 and the result analyzing unit 6.

In Step S7, the operating input acquisition unit 24 decides whether or not an instruction has been given to execute the target molecule inferring process.

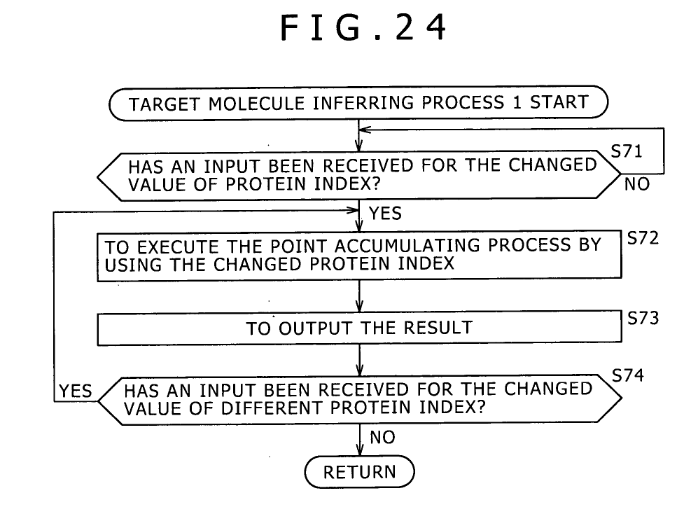

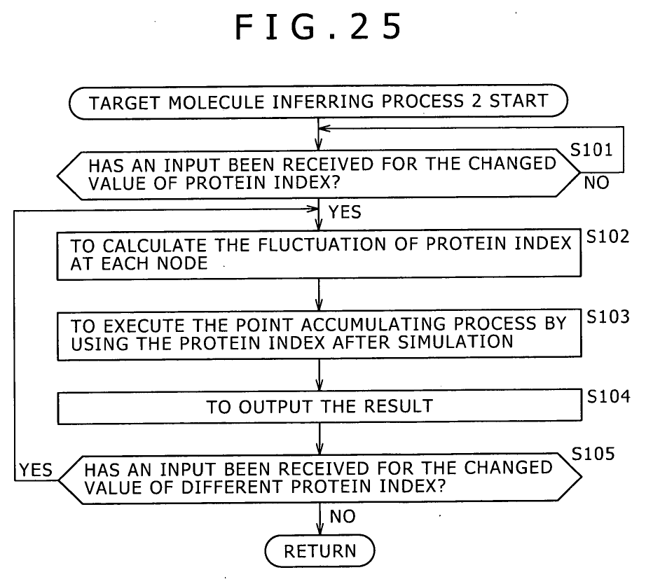

In Step S8, the target molecule inferring unit 27 performs the process to infer the target molecule according to the flow sheets shown in FIG. 24 or 25 (mentioned later), if it is judged in Step S7 that an instruction has been issued to execute the process of inferring the target molecule.

In Step S9, the result analyzing unit 6 decides whether or not an instruction has been given to analyze the result of analysis of proteins which has been supplied from the result output unit 28 of the protein information analyzing unit 4, if it is decided in Step S7 that no instruction has been issued to execute the process of inferring the target molecule or after completion of the processing Step S8.

In Step S10, the result analyzing unit 6 chronologically analyzes the result of protein analysis if it is judged in Step S9 that an instruction has been issued to analyze the result of protein analysis. The procedure for analysis includes accumulating chronologically the result of accumulation of the score for cellular function of the same test subject, analyzing the chronological changes, confirming whether or not the target protein has decreased as the result of medication to the test subject during the prescribed period, and confirming the effect on other proteins due to medication in the prescribed period or the effect on other cellular functions.

Step S11 is to decide whether or not an instruction has been issued to terminate the processing if it is judged in Step S9 that an instruction has been issued to analyze the result of protein analysis or after completion of the processing in Step S10.

The process returns to Step S7 if it is decided in Step S11 that no instruction for processing has been received from the user, and the steps after S7 are repeated. The process ends if it is judged in Step S11 that an instruction for processing has been received from the user.

The foregoing processing gives the score of cellular function in response to the amount of expression for individual molecule sets classified according to interactions between two molecules. The score of cellular function permits one to infer the target molecule and to analyze chronologically the result of protein analysis.

In what follows, the process for point accumulation to be performed in Step S5 shown in FIG. 22 will be described with reference to the flow chart shown in FIG. 23.

In Step S41, the point accumulating unit 22 extracts one of the molecule sets of NN-type, PN-type, or PP-type, which involves the proteins whose expression has been detected.

In Step S42, the point accumulating unit 22 extracts the factor, which has been set up by the factor setting unit 23, according to the classification of the molecule sets (NN-type, PN-type, or PP-type) and the presence or absence of the molecular bond.

In Step S43, the point accumulating unit 22 detects whether each of cellular functions corresponding to the molecule sets is promoted or suppressed, by referencing the information about the relation between the molecule set and the cellular function shown in FIGS. 3 to 14 which is stored in the protein information database 3.

Each of the cellular functions is associated with the molecule set as explained with reference to FIGS. 15 to 17.

In Step S44, the point accumulating unit 22 multiplies by a factor the value as the base of the score (said value being obtained as explained with reference to FIG. 18) for the cellular function corresponding to the molecule set, thereby calculating the score for the cellular function.

In Step S45, the point accumulating unit 22 decides whether or not the score has been added to all the molecule sets. If it is judged in Step S45 that the score is not yet added to all the molecule sets, the step returns to Step S41 and subsequent steps are repeated.

If it is decided in Step S45 that the score has been added to all the molecule sets, the point accumulating unit 22 performs accumulation for each cellular function in Step S46, and the step returns to Step S5 and proceeds to Step S6 (shown in FIG. 22).

The above-mentioned process accumulates the score for each cellular function, thereby allowing one to know which cellular function is promoted or suppressed in the sample cells.

In what follows, the target molecule inferring process 1 to be performed in Step S8 shown in FIG. 22 will be described with reference to the flow chart shown in FIG. 24.

The target molecule inferring process 1 infers the target molecule based only on the changed value of the protein index, without using the molecule network.

In Step S71, the operating input acquisition unit 24 decides whether or not it has received an input for the changed value of the protein index. If the operating input acquisition unit 24 decides in Step S71 that it has not yet received an input for the changed value of the protein index, it repeats the process in Step S71 until it judges that it has received an input for the changed value of the protein index.

In Step S72, the operating input acquisition unit 24 sends the value of the protein index entered to the target molecule inferring unit 27 if it is judged in Step S71 that it has received an input for the changed value of the protein index. The target molecule inferring unit 27 sends the changed value of the protein index entered to the point accumulating unit 22, thereby causing the point accumulating unit 22 to accumulate the point by using the changed protein index as explained with reference to FIG. 19 in the same way as explained with reference to FIG. 23. The point accumulating unit 22 performs the process of accumulating the point by using the changed protein index and sends the result to the result output unit 28.

In Step S73, the result output unit 28 sends the result of calculation which supplied from the point accumulating unit 22 to the result output unit 28 and the target molecule inferring unit 27.

In Step S74, the operating input acquisition unit 24 decides whether or not it has received an input for the changed value of different protein index. If the operating input acquisition unit 24 decides in Step S74 that it has received an input for the changed value of different protein index, the process returns to Step S72 and the subsequent processes are repeated. If the operating input acquisition unit 24 judges in Step S74 that it has not yet received an input for the changed value of different protein index, the process returns to Step S8 shown in FIG. 22 and proceeds to Step S9.

The foregoing process performs point accumulation by using the changed protein index as explained with reference to FIG. 19, and permits one to infer what cellular function is promoted or suppressed when what protein decreases in amount of expression as explained with reference to FIG. 20. This makes it possible to infer the candidate of the target molecule for anticancer agents by seeking a protein or a combination of proteins which is most effective for the desired cellular function.

In what follows, the target molecule inferring process 2 to be performed in Step S8 shown in FIG. 22 will be described with reference to the flow chart shown in FIG. 25.

The target molecule inferring process 2 infers the target molecule by simulating the changed value of protein index for a plurality of molecules by using the molecule network.

In Step S101, the operating input acquisition unit 24 decides whether or not it has received an input for the changed value of the protein index. If the operating input acquisition unit 24 decides in Step S101 that it has not yet received an input for the changed value of the protein index, it repeats the process in Step S101 until it judges that it has received an input for the changed value of the protein index.

In Step S101, the operating input acquisition unit 24 sends the value of the protein index entered to the target molecule inferring unit 27 if it is decided in Step S101 that it has received an input for the changed value of the protein index. The target molecule inferring unit 27 sends the changed value of the protein index entered to the network building unit 26, thereby causing the network building unit 26 to calculate the variation of the protein index at individual nodes that occurs when the value of the prescribed protein index is changed in the molecule network built up by the network building unit 26. The network building unit 26 calculates the variation of the protein index at each node based on the changed value of the protein index supplied, and sends the result to the point accumulating unit 22.

It is desirable to have a means for considering the presence of nodes under influence of more than one route for the increase or decrease of the protein index at a certain node.

In Step S103, the point accumulating unit 22 accumulates the point by using the protein index after simulation in the same way as explained with reference to FIG. 23.

In Step S104, the result output unit 28 sends the result of calculation (which has been received from the point accumulating unit 22) to the result output unit 28 and the target molecule inferring unit 27.

In Step S105, the operating input acquisition unit 24 decides whether or not it has accepted the input of the changed value of the different protein index. If it is decided in Step S105 that the input of the changed value of the different protein index has been accepted, the process returns to Step S102 and the subsequent process is repeated. If it is decided in Step S105 that the input of the changed value of the different protein index has not been accepted, the process returns to Step S8 shown in FIG. 22 and then proceeds to Step S9.

The above-mentioned process accumulates the score for cellular function once again by using the result of simulation by means of the molecule network, thereby allowing one to infer the target molecule.

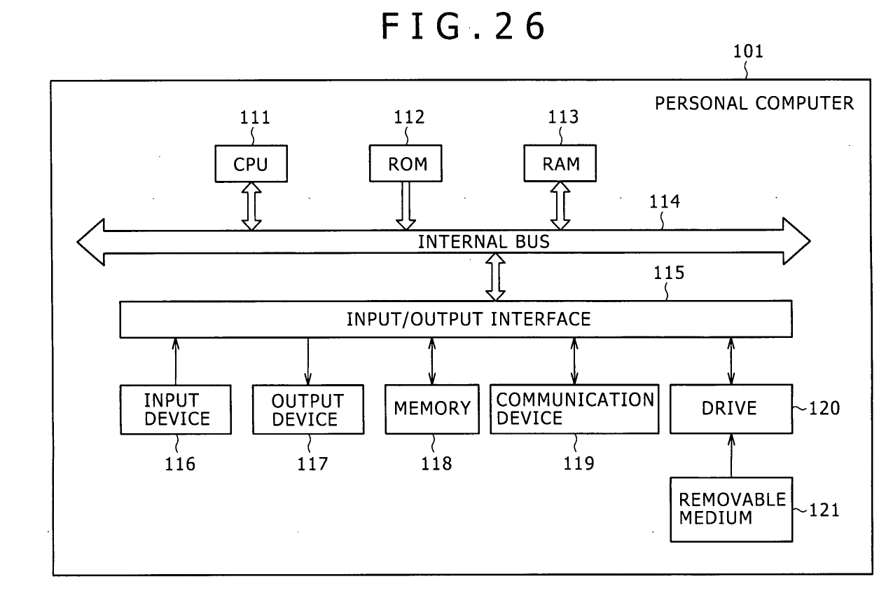

A series of processes mentioned above may be implemented by means of hardware or software. At least part of the above-mentioned process may be carried out by means of the personal computer 101 shown in FIG. 26.

In FIG. 26, the CPU (Central Processing Unit) 111 executes various processes according to the program stored in the ROM (Read Only Memory) 112 or the program loaded from the memory 118 to the RAM (Random Access Memory) 113. The RAM 113 also stores data necessary for the CPU 111 to perform various processes.

The CPU 111, the ROM 112, and the RAM 113 are connected to one another through the internal bus 114, which is connected to the input/output interface 115.

The input/output interface 115 is connected to the input device 116 such as keyboard and mouse, the output device 117 such as display and speaker, the memory unit 118 such as hard disk, and the communication unit 119 such as modem and terminal adaptor. The communication unit 119 performs communications through various networks including telephone circuit and CATV.

The input/output interface 115 is connected to the drive 120 according to need. The drive 120 may be equipped with the removable medium 121, such as magnetic disc, optical disc, magneto-optical disc, and semiconductor memory. The computer program is read out from the drive 120 and then installed in the memory 118 according to need.

In the case where software is used for processing, the program constituting the software is installed from the network or recording medium.

The recording medium may be the ROM 112 in which the program is recorded or the hard disc included in the memory device 118. In this case the ROM 112 and the hard disc are built into the personal computer delivered to the user. The program may also be recorded in the removal medium 121, which is distributed to the user separately from the computer proper.

In this specification, the steps for the program recorded in the recording medium may be carried out chronologically in the order listed; however, they may also be carried out in parallel or independently.

In this specification, the term “system” denotes an entire apparatus including a plurality of devices.

Incidentally, the embodiments of the present invention are not limited to those mentioned above; they may be modified variously without departing from the scope of the present invention.

What is claimed is:

1. An information processing apparatus which comprises:
acquisition means that acquires the amount of the molecules for detection which have been produced by control cells and sample cells;
arithmetic means that receives from said acquisition means the information about the amount of the molecules for detection which have been produced by said control cells and said sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection; and
output control means that controls the output of the score which has been calculated by the arithmetic means for the cellular function.

2. The information processing apparatus as defined in claim 1, wherein the acquisition means acquires the amount of the molecules for detection which have been produced by the control cells and the sample cells, according to the amount of the nucleic acid which has been expressed in response to the molecules for detection which have been collected from the control cells and the sample cells.

3. The information processing apparatus as defined in claim 1, wherein the combination of the two molecules for detection is classified into the following five categories according to the interrelation between the two molecules; the first category applicable to two molecules which suppress each other, the second category applicable to two molecules the first one of which promotes the second one and the second one of which suppresses the first one, the third category applicable to two molecules which promote each other, the fourth category applicable to two molecules only one of which promotes the other, and the fifth category applicable to two molecules only one of which suppresses the other.

4. The information processing apparatus as defined in claim 1, wherein the arithmetic means calculates the score for the cellular functions by accumulating for each cellular function those values which are obtained by giving the score based on the amount of the molecules for detection which have been produced in the control cells and the sample cells to the cellular functions relating to the mutual promotion or suppression between the two molecules for detection which belong to the first to third categories out of said five categories and then multiplying a prescribed factor.

5. The information processing apparatus as defined in claim 4, wherein the prescribed factor is established such that it takes on the largest value for the cellular function relating to the first category of the first to third categories out of the five categories and it also takes on the smallest value for the cellular function relating to the third category of the first to third categories out of the five categories.

6. The information processing apparatus as defined in claim 4, wherein the prescribed factor is larger than 1 when the two molecules for detection have a molecular bond.

7. The information processing apparatus as defined in claim 1, further comprises
storage means that stores in a table form the information about the combination of the two molecules for detection which are classified into any of the five categories and the cellular function relating to the mutual promotion or suppression of the two molecules for detection.

8. The information processing apparatus as defined in claim 1, further comprises
estimating means that estimates the score for the cellular function when there is any change in the amount of the molecules for detection which have been produced in the control cells and the sample cells after it has been acquired by the acquisition means.

9. The information processing apparatus as defined in claim 8, further comprises
network building means that builds a network for the information about the interrelation of the molecules for detection, wherein
said estimating means calculates the effect of change in the amount of the molecules for detection which have been produced on other molecules based on the network which has been built by the network building means, thereby estimating the score for the cellular function.

10. The information processing apparatus as defined in claim 1, further comprises
analyzing means that analyzes the change with time of the cellular function based on the score for the cellular function, with its output being controlled by the output control means.

11. An information processing method for an information processing apparatus that analyzes the cellular function relating to mutual promotion or suppression between two molecules to be detected, said method comprising the steps of:
acquiring the amount of the molecules for detection which have been produced in the control cells and the sample cells; and
receiving the information about the amount of the molecules for detection which have been produced in the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection.

12. A program to be executed by a computer to analyze the cellular function relating to mutual promotion or suppression between two molecules to be detected, said program comprising the steps of:
acquiring the amount of the molecules for detection which have been produced in the control cells and the sample cells, and
receiving the information about the amount of the molecules for detection which have been produced in the control cells and the sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection.

13. A recording medium which records the program as defined in claim 12.

14. An information processing system comprises:
an analyzing unit that analyzes the amount of the molecules for detection which have been produced in the control cells and the sample cells; and
an information processing apparatus that analyzes the information about the cellular function relating to the mutual promotion or suppression of the two molecules for detection,
wherein said information processing apparatus includes:
acquisition means that acquires the amount of the molecules for detection which have been produced by control cells and sample cells;
arithmetic means that receives from said acquisition means the information about the amount of the molecules for detection which have been produced by said control cells and said sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection; and
output control means that controls the output of the score which has been calculated by said arithmetic means for the cellular function.

15. An information processing apparatus comprises:
an acquisition unit that acquires the amount of the molecules for detection which have been produced by control cells and sample cells;
an arithmetic unit that receives from said acquisition unit the information about the amount of the molecules for detection which have been produced by said control cells and said sample cells, thereby calculating the score that indicates whether one of the two molecules for detection promotes or suppresses the other and also indicates whether the cellular function is promoted or suppressed depending on the combination of cellular functions for the mutual promotion or suppression between the two molecules for detection; and
an output control unit that controls the output of the score which has been calculated by the arithmetic unit for the cellular function.
